(19) United States
(12) Patent Application Publication
(10) Pub. No.: US 2016/0281125 A1
(43) Pub. Date: Sep. 29, 2016

(54) ANTIMICROBIAL COMPOUND SUSCEPTIBILITY TEST

(71) Applicant: AdvanDx, Inc., Woburn, MA (US)

(72) Inventors: Melissa K. Deck, Somerville, MA (US); Dennis H. Langer, Naples, FL (US)

(73) Assignee: AdvanDx, Inc., Woburn, MA (US)

(21) Appl. No.: 15/083,798

(22) Filed: Mar. 29, 2016

Related U.S. Application Data
(63) Continuation of application No. PCT/US2014/058146, filed on Sep. 29, 2014.
(60) Provisional application No. 61/884,204, filed on Sep. 30, 2013.

(51) Int. Cl. C12Q 1/18; A61K 31/546; A61K 31/407

(52) U.S. Cl. CPC C12Q 1/18; A61K 31/407; A61K 31/546

(57) ABSTRACT

Methods of determining whether a target bacteria is susceptible to an antimicrobial compound are provided. In some embodiments the methods comprise providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining the level of lysis and/or the level of remaining intact cells present in the antimicrobial compound-exposed target bacterial sample whether the cell-wall disruption condition lyses target bacterial cells present in the antimicrobial compound-exposed target bacterial sample; wherein the method is performed such that the level of lysis and/or remaining intact cells is determined without determining lysis or non-lysis on a cell-by-cell basis. In some embodiments target bacteria are not immobilized during the exposure to cell-wall disruption conditions. In some embodiments the methods further comprise comparing the level of lysis and/or the level of remaining intact cells present in the antimicrobial compound-exposed target bacterial sample to a reference level to score the sample as sensitive or resistant to the at least one antimicrobial compound. In some embodiments the method does not comprise detecting the presence or absence of at least one target bacteria protein and/or at least one target bacteria nucleic acid. Methods of treating a bacterial infection in a subject are also provided. Kits and systems that may be used to, for example, practice the methods are also provided.

### RELATED APPLICATIONS

This application is a continuation of International Application No. PCT/US2014/058146, filed Sep. 29, 2014; which claims priority to U.S. Provisional Patent Application No. 61/884,204, filed Sep. 30, 2013, each of which is hereby incorporated herein by reference in its entirety.

### INTRODUCTION

The emergence and spread of antimicrobial-resistant bacteria is a serious global health threat. The development is associated with extensive and increasing use of antimicrobial agents. Coupled with the limited development of new antimicrobial agents, this has drastically limited the treatment options for resistant pathogens. Infections with resistant pathogens are associated with higher mortality, morbidity, and health care costs. Early targeted antimicrobial compound treatment is an important prognostic factor especially in the seriously ill patients.

There is evidence that the avoidance of inappropriate broad-spectrum antimicrobial therapy can prevent antimicrobial resistance. At the same time, deployment of a broad spectrum therapy as early as possible will improve patient outcomes. Accordingly, there is a demand for methods that can determine antimicrobial compound susceptibility and enable early targeted therapy as early in a treatment as possible. Conventional culture-based methods, such as disc diffusion test, E-test (BioMérieux), broth or agar dilution, and others that are used to determine the minimum inhibitory concentration (MIC) of antimicrobial compounds are time-consuming end-point methods. Commercial instruments such as Phoenix 100 (BD biosciences) and Vitek 2 (BioMérieux) allow automation and reduce hands-on and incubation time. Both instruments operate with colorimetric or fluorimetric indicators for bacterial identification and estimation of growth rate. The average time for identification is 4.3 h for Phoenix and 3-5.7 h for Vitek 2, depending on bacterial type, while the mean time for AST is 12.1 h and 9.8 h, respectively. Translating these times-to-results into clinical practice implies that a switch from broad-spectrum antimicrobial compound therapy to narrow-spectrum targeted therapy will only be accomplished the following working day.

In order to improve sensitivity and speed, several biosensors based on either chip-calorimetry, electrical conductivity, millifluidic droplet analyzer, or utilizing surface plasmon resonance have been developed. Presently, these techniques are limited by single-sample analysis and the requirement for specialized technical personal. The use of molecular methods such as real-time polymerase chain reaction (PCR), mass spectrometry, micro array, and flow cytometry has now been developed for rapid bacterial identification or AST because of their high sensitivity and promptness. Bacterial identification of a wide range of bacteria can be performed within minutes using mass spectrometry, and flow cytometry permits prediction of antimicrobial susceptibility within 90-120 minutes. However, these techniques require expensive equipment, special probes, and/or skilled personnel.

U.S. Patent Application Publication No. 2012/0122831 describes methods that include the application of shear stress and/or chemical stress to bacteria in the presence of an antimicrobial compound. The shear and/or chemical stress catalyzes the biochemical pathways that repair stress-induced damage to the cells. These pathways are the targets of certain antimicrobial compounds and therefore repair is inhibited in the presence of those antimicrobial compounds. For example, the methods may comprise immobilizing bacteria to a solid support, contacting the bacteria with an agent comprising a reporter moiety, subjecting the immobilized bacteria to a stressor in the presence or absence of an antimicrobial compound, and detecting a signal from the reporter moiety. Detection of a signal indicates that the agent has been delivered into the cell as a result of cell damage. Thus, the methods rely on detection of cellular damage in intact cells, based on the differential ability of the agent comprising a reporter moiety to label damaged intact cells as compared to undamaged intact cells.

U.S. Patent Application Publication No. 2013/0008793 describes methods that include providing a test sample containing a bacteria; adding an antimicrobial compound to the test sample to inhibit cell wall synthesis; executing dielectrophoresis to the test sample and observing morphologic changes of the bacteria in the test sample; and determining whether the bacteria is resistant to the antimicrobial compound according to the morphologic changes of the bacteria. Thus, the methods rely on detection of morphologic changes in intact cells.

Marlene Fredborg, et al., Clin. Microbiol., doi:10.1128/JCM.00440-13 (17 Apr. 2013), has described an oCelloScope system that uses digital time-lapse microscopy scanning through a fluid sample generating series of images. The images are processed to observe bacterial growth at a single cell level. Thus, the disclosed methods rely on detection of changes in intact cells.

Santiso et al., BMC Microbiology 2011, 11:191 is titled “A rapid in situ procedure for determination of bacterial susceptibility or resistance to antimicrobial compounds that inhibit peptidoglycan biosynthesis.” The article describes that “Cells incubated with the antimicrobial compound were embedded in an agarose microgel on a slide, incubated in an adapted lysis buffer, stained with a DNA fluorochrome, SYBR Gold and observed under fluorescence microscopy.” According to the article, “The lysis affects the cells differentially, depending on the integrity of the wall. If the bacterium is susceptible to the antimicrobial compound, the weakened cell wall is affected by the lysing solution so the nucleoid of DNA contained inside the bacterium is released and spread. Alternatively, if the bacterium is resistant to the antimicrobial compound, it is practically unaffected by the lysis solution and does not liberate the nucleoid, retaining its normal morphological appearance.” One aspect of the disclosed methods is their use of detection of microgranular-fibrilar extracellular background to identify susceptible strains. That material is formed of DNA fragments released by lysed susceptible bacteria in the course of the steps of the methods. Thus, the disclosed methods comprise immobilizing cells before treatment with lysis conditions and also utilize specific detection of debris released from lysed cells.

There is a need for new methods, systems, and kits for assaying bacterial susceptibility to antimicrobial compound agents. Methods, systems, and kits that provide at least one of a useful total time required for the assay and an easy to implement assay format will be particularly useful, although these aspects are not necessarily required for a method, system, or kit to be useful. This disclosure meets the need in the art to provide new methods, systems, and kits for assaying bacterial susceptibility to antimicrobial compound agents.

### SUMMARY

In general, the methods, systems, and kits disclosed herein are based in part on the observation of the inventors that a cell-wall disruption condition may be applied to bacterial cells that have been exposed to an antimicrobial compound to selectively lyse bacterial cells that are susceptible to the antimicrobial compound. This observation enabled the inventors to provide various methods, systems, and kits that may be used for bacterial antimicrobial compound susceptibility testing and are disclosed herein.

In a first aspect this disclosure provides methods of determining whether a target bacteria is susceptible to an antimicrobial compound. In some embodiments the methods comprise determining whether the target bacteria is sensitive to the antimicrobial compound. In some embodiments the methods comprise determining whether the target bacteria is resistant to the antimicrobial compound. In some embodiments the methods comprise determining whether the target bacteria is sensitive and/or resistant to the antimicrobial compound.

In some embodiments the methods comprise providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining whether the cell-wall disruption condition lyses target bacterial cells present in the antimicrobial compound-exposed target bacterial sample. In some embodiments the antimicrobial compound-exposed target bacterial sample is exposed to a cell-wall disruption condition without immobilizing antimicrobial compound-exposed target bacteria.

In some embodiments the methods comprise providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining the level of lysis and/or the level of remaining intact cells present in the antimicrobial compound-exposed target bacterial sample cells present in the antimicrobial compound-exposed target bacterial sample; wherein the method is performed such that the level of lysis and/or remaining intact cells is determined without determining lysis or non-lysis on a cell-by-cell basis.

In some embodiments the methods further comprise comparing the level of lysis and/or the level of remaining intact cells present in the antimicrobial compound-exposed target bacterial sample to a reference level to score the sample as sensitive or resistant to the at least one antimicrobial compound.

In some embodiments if the level of lysis present in the antimicrobial compound-exposed target bacterial sample is at or above a reference level and/or the level of remaining intact cells present in the antimicrobial compound-exposed target bacterial sample is at or below a reference level, the target bacteria are scored as sensitive to the antimicrobial compound if the cell-wall disruption condition lyses target bacterial cells present in the antimicrobial compound-exposed target bacterial sample, the target bacteria is susceptible to the at least one antimicrobial compound.

In some embodiments if the level of lysis present in the antimicrobial compound-exposed target bacterial sample is not at or above a reference level and/or the level of remaining intact cells present in the antimicrobial compound-exposed target bacterial sample is not at or below a reference level, the target bacteria are scored as resistant to the antimicrobial compound if the cell-wall disruption condition does not lyse target bacterial cells present in the antimicrobial compound-exposed target bacterial sample, the target bacteria is not susceptible to the at least one antimicrobial compound.

In some embodiments the target bacteria are not immobilized during the exposure to cell-wall disruption conditions.

In some embodiments the methods do not comprise detecting the presence or absence of at least one target bacteria protein and/or at least one target bacteria nucleic acid. In some embodiments the sample comprising the target bacteria is a primary sample. In some embodiments the sample comprising the target bacteria is an in vitro cultured sample.

In some embodiments the in vitro cultured sample is provided by obtaining a sample comprising the target bacteria from a subject and culturing target bacteria in the subject sample to provide the in vitro cultured sample.

In some embodiments the target bacteria is Gram-negative. In some embodiments the target bacteria is rod-shaped. In some embodiments the target bacteria is a member of the family Enterobacteriaceae. In some embodiments the target bacteria is a non-fermenter bacteria.

In some embodiments the antimicrobial compound is a bactericidal antimicrobial compound. In some embodiments the antimicrobial compound comprises a β-lactam ring. In some embodiments the antimicrobial compound is a carbapenem. In some embodiments the antimicrobial compound is selected from colistin or a derivative thereof, tigecycline or a derivative thereof, a cephalosporin or a derivative thereof, a carbapenem or a derivative thereof, cefoxitin or a derivative thereof, and fosfomycin or a derivative thereof.

In some embodiments the sample is maintained in the presence of a concentration of the at least one antimicrobial compound that is at least the minimum inhibitory concentration of the at least one antimicrobial compound. In some embodiments the sample is maintained in the presence of the antimicrobial compound for about two hours or less.

In some embodiments the cell-wall disruption condition comprises at least one of a detergent, a physical means of disrupting cells, alkaline conditions, a chemical cell-wall disruption agent, and an enzyme. In some embodiments the cell-wall disruption condition comprises a detergent and a physical means of disrupting cells. In some embodiments the detergent is selected from at least one of Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

In some embodiments, if the cell-wall disruption condition lyses target bacterial cells present in the antimicrobial compound-exposed target bacterial sample, the target bacteria is susceptible to the antimicrobial compound. In some embodiments, if the cell-wall disruption condition does not lyse target bacterial cells present in the antimicrobial compound-exposed target bacterial sample, the target bacteria is not susceptible to the antimicrobial compound. In some embodiments the methods further comprise determining the extent of lysis of target bacterial cells present in the antimicrobial compound-exposed target bacterial sample.

In some embodiments determining whether the cell-wall disruption condition lyses target bacterial cells present in the antimicrobial compound-exposed target bacterial sample does not comprise counting target bacterial cells

In some embodiments, determining whether the cell-wall disruption condition lyses target bacterial cells present in the antimicrobial compound-exposed target bacterial sample comprises detecting intact (unlysed) target bacterial cells. In some embodiments, determining whether the cell-wall disruption condition lyses target bacterial cells present in the antimicrobial compound-exposed target bacterial sample comprises detecting lysed target bacterial cells. In some embodiments, determining whether the cell-wall disruption condition lyses target bacterial cells present in the antimicrobial compound-exposed target bacterial sample comprises detecting intact (unlysed) target bacterial cells and detecting lysed target bacterial cells. In some embodiments, determining whether the cell-wall disruption condition lyses target bacterial cells present in the antimicrobial compound-exposed target bacterial sample comprises detecting intact (unlysed) target bacterial cells and does not comprise detecting lysed target bacterial cells. In some embodiments, determining whether the cell-wall disruption condition lyses target bacterial cells present in the antimicrobial compound-exposed target bacterial sample comprises detecting lysed target bacterial cells and does not comprise detecting intact (unlysed) target bacterial cells. In some embodiments, detecting intact (unlysed) target bacterial cells comprises counting the intact (unlysed) target bacterial cells. In some embodiments, detecting intact (unlysed) target bacterial cells comprises staining the intact (unlysed) target bacterial cells with a marker that enables specific identification of intact (unlysed) target bacterial cells.

In some embodiments the methods further comprise providing a sample comprising the target bacteria; maintaining the sample in the absence of the antimicrobial compound to provide an antimicrobial compound-negative control target bacterial sample; exposing the antimicrobial compound-negative control target bacterial sample to the cell-wall disruption condition; and determining the level of lysis and/or the level of remaining intact cells present in the antimicrobial compound-negative control target bacterial sample. In some embodiments the methods further comprise comparing the level of lysis and/or the level of remaining intact cells present in the antimicrobial compound-exposed target bacterial sample to the level of lysis and/or the level of remaining intact cells present in the antimicrobial compound-negative target bacterial sample. In some embodiments of the methods of this disclosure a plurality of concentrations of an antimicrobial compound are assayed, either in parallel and/or in series. Accordingly, in some embodiments the methods comprise determining whether a target bacteria is susceptible to an antimicrobial compound by a method comprising: providing a plurality of samples comprising the target bacteria; maintaining the plurality of samples in the presence of a plurality of concentrations of an antimicrobial compound to provide a plurality of antimicrobial compound-exposed target bacterial samples; exposing the plurality of antimicrobial compound-exposed target bacterial samples to a cell-wall disruption condition; and determining whether the cell-wall disruption condition lyses target bacterial cells present in the plurality of antimicrobial compound-exposed target bacterial samples. Additionally, in some embodiments the methods comprise determining whether a target bacteria is susceptible to an antimicrobial compound by a method comprising: providing a plurality of samples comprising the target bacteria; maintaining the plurality of samples in the presence of a plurality of concentrations of an antimicrobial compound to provide a plurality of antimicrobial compound-exposed target bacterial samples; exposing the plurality of antimicrobial compound-exposed target bacterial samples to a cell-wall disruption condition; and determining the level of lysis and/or the level of remaining intact cells present in the antimicrobial compound-exposed target bacterial sample; wherein the method is performed such that the level of lysis and/or remaining intact cells is determined without determining lysis or non-lysis on a cell-by-cell basis.

In some embodiments the plurality of concentrations of an antimicrobial compound comprises a sample maintained in the absence of the antimicrobial compound. In some embodiments the methods further comprise determining the level of lysis and/or the level of remaining intact cells present in the plurality of antimicrobial compound-exposed target bacterial samples. In some embodiments the methods further comprise comparing the level of lysis and/or the level of remaining intact cells present in the plurality of antimicrobial compound-exposed target bacterial samples across the range of tested antimicrobial compound concentrations. In some embodiments the methods further comprise determining the concentration of the antimicrobial compound that causes lysis at or above a reference level of target bacterial cells present in the sample after exposing the sample to the cell-wall disruption condition. In some embodiments the methods further comprise determining the concentration of the antimicrobial compound that causes lysis at or above a reference level of target bacterial cells present in the sample after exposing the sample to the cell-wall disruption condition.

In some embodiments of the methods of this disclosure a plurality of different densities of target bacterial cells are assayed, either in parallel and/or in series. Such embodiments may allow, for example, a determination of the effect of cell density on the antimicrobial activity of a tested compound. Accordingly, also provided are methods of determining whether a target bacteria is susceptible to an antimicrobial compound, comprising: providing a plurality of samples comprising different densities of the target bacteria; maintaining the plurality of samples in the presence of an antimicrobial compound to provide a plurality of antimicrobial compound-exposed target bacterial samples; exposing the plurality of antimicrobial compound-exposed target bacterial samples to a cell-wall disruption condition; and determining whether the cell-wall disruption condition lyses target bacterial cells present in the plurality of antimicrobial compound-exposed target bacterial samples. In some embodiments the methods further comprise determining the level of lysis of target bacterial cells present in the plurality of antimicrobial compound-exposed target bacterial samples. In some embodiments the methods further comprise comparing the level of lysis of target bacterial cells present in the plurality of antimicrobial compound-exposed target bacterial samples across the range of tested target bacterial cell densities. In some embodiments the methods further comprise determining the threshold density of target bacterial cells that is lysed in at least a threshold proportion after exposing the sample to the cell-wall disruption condition.

In some embodiments the time elapsed between the beginning of maintaining the sample in the presence of the antimicrobial compound to the determination of whether the cell-wall disruption condition lyses target bacterial cells present in the antimicrobial compound-exposed target bacterial sample is three hours of less.

In another aspect this disclosure also provides methods of treating a bacterial infection in a subject. The methods may comprise determining that a target bacteria is sensitive to an antimicrobial compound by a method disclosed herein and administering a therapeutically effective amount of the antimicrobial compound to the subject to thereby treat the bacterial infection in the subject. In some embodiments the antimicrobial compound-exposed target bacterial sample is exposed to a cell-wall disruption condition without immobilizing antimicrobial compound-exposed target bacteria.

In some embodiments of the treatment methods, determining that a target bacteria is sensitive to an antimicrobial compound comprises providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining whether the cell-wall disruption condition lyses target bacterial cells present in the antimicrobial compound-exposed target bacterial sample. In some embodiments the method comprises the antimicrobial compound-exposed target bacterial sample is exposed to a cell-wall disruption condition without immobilizing antimicrobial compound-exposed target bacteria.

In some embodiments of the treatment methods, determining that a target bacteria is sensitive to an antimicrobial compound comprises providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining the level of lysis and/or the level of remaining intact cells present in the antimicrobial compound-exposed target bacterial sample whether the cell-wall disruption condition lyses target bacterial cells present in the antimicrobial compound-exposed target bacterial sample; wherein the method is performed such that the level of lysis and/or remaining intact cells is determined without determining lysis or non-lysis on a cell-by-cell basis.

In some embodiments of the treatment methods, determining that a target bacteria is sensitive to an antimicrobial compound further comprises comparing the level of lysis and/or the level of remaining intact cells present in the antimicrobial compound-exposed target bacterial sample to a reference level to score the sample as sensitive or resistant to the at least one antimicrobial compound.

In some embodiments of the treatment methods, if the level of lysis present in the antimicrobial compound-exposed target bacterial sample is at or above a reference level and/or the level of remaining intact cells present in the antimicrobial compound-exposed target bacterial sample is at or below a reference level, the target bacteria are scored as sensitive to the antimicrobial compound if the cell-wall disruption condition lyses target bacterial cells present in the antimicrobial compound-exposed target bacterial sample, the target bacteria is susceptible to the at least one antimicrobial compound.

In some embodiments of the treatment methods, if the level of lysis present in the antimicrobial compound-exposed target bacterial sample is not at or above a reference level and/or the level of remaining intact cells present in the antimicrobial compound-exposed target bacterial sample is not at or below a reference level, the target bacteria are scored as resistant to the antimicrobial compound if the cell-wall disruption condition does not lyse target bacterial cells present in the antimicrobial compound-exposed target bacterial sample, the target bacteria is not susceptible to the at least one antimicrobial compound.

In some embodiments of the treatment methods, the target bacteria are not immobilized during the exposure to cell-wall disruption conditions.

In some embodiments of the treatment methods, the methods do not comprise detecting the presence or absence of at least one target bacteria protein and/or at least one target bacteria nucleic acid.

In some embodiments of the treatment methods, the sample comprising the target bacteria is a primary sample. In some embodiments the sample comprising the target bacteria is an in vitro cultured sample. In some embodiments of the treatment methods, the in vitro cultured sample is provided by obtaining a sample comprising the target bacteria from a subject and culturing target bacteria in the subject sample to provide the in vitro cultured sample.

In some embodiments of the treatment methods, the target bacteria is Gram-negative. In some embodiments of the treatment methods, the target bacteria is rod-shaped. In some embodiments of the treatment methods, the target bacteria is a member of the family Enterobacteriaceae. In some embodiments of the treatment methods, the target bacteria is a non-fermenter bacteria.

In some embodiments of the treatment methods, the antimicrobial compound is a bactericidal antimicrobial compound. In some embodiments of the treatment methods, the antimicrobial compound comprises a β-lactam ring. In some embodiments of the treatment methods, the antimicrobial compound is a carbapenem. In some embodiments of the treatment methods, the antimicrobial compound is selected from colistin or a derivative thereof, tigecycline or a derivative thereof, a cephalosporin or a derivative thereof, a carbapenem or a derivative thereof, cefoxitin or a derivative thereof, and fosfomycin or a derivative thereof.

In some embodiments of the treatment methods, the sample is maintained in the presence of a concentration of the at least one antimicrobial compound that is at least the minimum inhibitory concentration of the at least one antimicrobial compound. In some embodiments the sample is maintained in the presence of the antimicrobial compound for about two hours or less.

In some embodiments of the treatment methods, the cell-wall disruption condition comprises at least one of a detergent, a physical means of disrupting cells, alkaline conditions, a chemical cell-wall disruption agent, and an enzyme. In some embodiments the cell-wall disruption condition comprises a detergent and a physical means of disrupting cells. In some embodiments the detergent is selected from at least one of Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

In some embodiments of the treatment methods, if the cell-wall disruption condition lyses target bacterial cells present in the antimicrobial compound-exposed target bacterial sample, the target bacteria is susceptible to the antimicrobial compound. In some embodiments, if the cell-wall disruption condition does not lyse target bacterial cells present in the antimicrobial compound-exposed target bacterial sample, the target bacteria is not susceptible to the antimicrobial compound. In some embodiments the methods further comprise determining the extent of lysis of target bacterial cells present in the antimicrobial compound-exposed target bacterial sample.

In some embodiments of the treatment methods, determining whether the cell-wall disruption condition lyses target bacterial cells present in the antimicrobial compound-exposed target bacterial sample does not comprise counting target bacterial cells.

In some embodiments of the treatment methods, determining whether the cell-wall disruption condition lyses target bacterial cells present in the antimicrobial compound-exposed target bacterial sample comprises detecting intact (unlysed) target bacterial cells. In some embodiments, determining whether the cell-wall disruption condition lyses target bacterial cells present in the antimicrobial compound-exposed target bacterial sample comprises detecting lysed target bacterial cells. In some embodiments, determining whether the cell-wall disruption condition lyses target bacterial cells present in the antimicrobial compound-exposed target bacterial sample comprises detecting intact (unlysed) target bacterial cells and detecting lysed target bacterial cells. In some embodiments, determining whether the cell-wall disruption condition lyses target bacterial cells present in the antimicrobial compound-exposed target bacterial sample comprises detecting intact (unlysed) target bacterial cells and does not comprise detecting lysed target bacterial cells. In some embodiments, determining whether the cell-wall disruption condition lyses target bacterial cells present in the antimicrobial compound-exposed target bacterial sample comprises detecting lysed target bacterial cells and does not comprise detecting intact (unlysed) target bacterial cells. In some embodiments, detecting intact (unlysed) target bacterial cells comprises counting the intact (unlysed) target bacterial cells. In some embodiments, detecting intact (unlysed) target bacterial cells comprises staining the intact (unlysed) target bacterial cells with a marker that enables specific identification of intact (unlysed) target bacterial cells.

In some embodiments the treatment methods further comprise providing a sample comprising the target bacteria; maintaining the sample in the absence of the antimicrobial compound to provide an antimicrobial compound-negative control target bacterial sample; exposing the antimicrobial compound-negative control target bacterial sample to the cell-wall disruption condition; and determining the level of lysis and/or the level of remaining intact cells present in the antimicrobial compound-negative control target bacterial sample. In some embodiments the methods further comprise comparing the level of lysis and/or the level of remaining intact cells present in the antimicrobial compound-exposed target bacterial sample to the level of lysis and/or the level of remaining intact cells present in the antimicrobial compound-negative target bacterial sample.

In some embodiments determining whether the cell-wall disruption condition lyses target bacterial cells present in the antimicrobial compound-exposed target bacterial sample does not comprise counting target bacterial cells.

In some embodiments of the treatment methods, the time elapsed between the beginning of maintaining the sample in the presence of the antimicrobial compound to the determination of whether the cell-wall disruption condition lyses target bacterial cells present in the antimicrobial compound-exposed target bacterial sample is three hours of less. In some embodiments, administering a therapeutically effective amount of the antimicrobial compound to the subject to thereby treat the bacterial infection in the subject is initiated within two hours of the beginning of maintaining the sample in the presence of the antimicrobial compound.

In another aspect this disclosure provides methods of screening a candidate compound to identify a compound having antimicrobial activity against a target bacteria. In some embodiments the methods comprise determining whether the target bacteria is sensitive to a candidate antimicrobial compound. In some embodiments the methods comprise determining whether the target bacteria is resistant to the candidate antimicrobial compound. In some embodiments the methods comprise determining whether the target bacteria is sensitive and/or resistant to the candidate antimicrobial compound.

In some embodiments the methods comprise providing a sample comprising the target bacteria; maintaining the sample in the presence of a candidate antimicrobial compound to provide a candidate antimicrobial compound-exposed target bacterial sample; exposing the candidate antimicrobial compound-exposed target bacterial sample to a cell-wall disruption condition; and determining whether the cell-wall disruption condition lyses target bacterial cells present in the candidate antimicrobial compound-exposed target bacterial sample. In some embodiments the candidate antimicrobial compound-exposed target bacterial sample is exposed to a cell-wall disruption condition without immobilizing candidate antimicrobial compound-exposed target bacteria.

In some embodiments the methods comprise providing a sample comprising the target bacteria; maintaining the sample in the presence of a candidate antimicrobial compound to provide a candidate antimicrobial compound-exposed target bacterial sample; exposing the candidate antimicrobial compound-exposed target bacterial sample to a cell-wall disruption condition; and determining the level of lysis and/or the level of remaining intact cells present in the candidate antimicrobial compound-exposed target bacterial sample; wherein the method is performed such that the level of lysis and/or remaining intact cells is determined without determining lysis or non-lysis on a cell-by-cell basis.

In some embodiments the methods further comprise comparing the level of lysis and/or the level of remaining intact cells present in the candidate antimicrobial compound-exposed target bacterial sample to a reference level to score the sample as sensitive or resistant to the at least one candidate antimicrobial compound.

In some embodiments if the level of lysis present in the candidate antimicrobial compound-exposed target bacterial sample is at or above a reference level and/or the level of remaining intact cells present in the antimicrobial compound-exposed target bacterial sample is at or below a reference level, the target bacteria are scored as sensitive to the candidate antimicrobial compound.

In some embodiments if the level of lysis present in the candidate antimicrobial compound-exposed target bacterial sample is not at or above a reference level and/or the level of remaining intact cells present in the antimicrobial compound-exposed target bacterial sample is not at or below a reference level, the target bacteria are scored as resistant to the antimicrobial compound.

In some embodiments the target bacteria are not immobilized during the exposure to cell-wall disruption conditions.

In some embodiments the methods do not comprise detecting the presence or absence of at least one target bacteria protein and/or at least one target bacteria nucleic acid. In some embodiments the sample comprising the target bacteria is a primary sample. In some embodiments the sample comprising the target bacteria is an in vitro cultured sample. In some embodiments the in vitro cultured sample is provided by obtaining a sample comprising the target bacteria from a subject and culturing target bacteria in the subject sample to provide the in vitro cultured sample.

In some embodiments the target bacteria is Gram-negative. In some embodiments the target bacteria is rod-shaped. In some embodiments the target bacteria is a member of the family Enterobacteriaceae. In some embodiments the target bacteria is a non-fermenter bacteria.

In some embodiments the candidate antimicrobial compound is a bactericidal antimicrobial compound. In some embodiments the candidate antimicrobial compound comprises a β-lactam ring. In some embodiments the candidate antimicrobial compound is a carbapenem. In some embodiments the candidate antimicrobial compound is selected from colistin or a derivative thereof, tigecycline or a derivative thereof, a cephalosporin or a derivative thereof, a carbapenem or a derivative thereof, cefoxitin or a derivative thereof, and fosfomycin or a derivative thereof.

In some embodiments the sample is maintained in the presence of a concentration of the at least one candidate antimicrobial compound that is at least the minimum inhibitory concentration of the at least one candidate antimicrobial compound. In some embodiments the sample is maintained in the presence of the candidate antimicrobial compound for about two hours or less.

In some embodiments the cell-wall disruption condition comprises at least one of a detergent, a physical means of disrupting cells, alkaline conditions, a chemical cell-wall disruption agent, and an enzyme. In some embodiments the cell-wall disruption condition comprises a detergent and a physical means of disrupting cells. In some embodiments the detergent is selected from at least one of Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

In some embodiments, if the cell-wall disruption condition lyses target bacterial cells present in the candidate antimicrobial compound-exposed target bacterial sample, the target bacteria is sensitive to the candidate antimicrobial compound and the candidate is identified as an antimicrobial compound. In some embodiments, if the cell-wall disruption condition does not lyse target bacterial cells present in the antimicrobial compound-exposed target bacterial sample, the target bacteria is resistant to the antimicrobial compound and the candidate is not identified as an antimicrobial compound.

In some embodiments determining whether the cell-wall disruption condition lyses target bacterial cells present in the candidate antimicrobial compound-exposed target bacterial sample does not comprise counting target bacterial cells.

In some embodiments, determining whether the cell-wall disruption condition lyses target bacterial cells present in the and the candidate is identified as an antimicrobial compound antimicrobial compound-exposed target bacterial sample comprises detecting intact (unlysed) target bacterial cells. In some embodiments, determining whether the cell-wall disruption condition lyses target bacterial cells present in the candidate antimicrobial compound-exposed target bacterial sample comprises detecting lysed target bacterial cells. In some embodiments, determining whether the cell-wall disruption condition lyses target bacterial cells present in the candidate antimicrobial compound-exposed target bacterial sample comprises detecting intact (unlysed) target bacterial cells and detecting lysed target bacterial cells. In some embodiments, determining whether the cell-wall disruption condition lyses target bacterial cells present in the candidate antimicrobial compound-exposed target bacterial sample comprises detecting intact (unlysed) target bacterial cells and does not comprise detecting lysed target bacterial cells. In some embodiments, determining whether the cell-wall disruption condition lyses target bacterial cells present in the candidate antimicrobial compound-exposed target bacterial sample comprises detecting lysed target bacterial cells and does not comprise detecting intact (unlysed) target bacterial cells. In some embodiments, detecting intact (unlysed) target bacterial cells comprises counting the intact (unlysed) target bacterial cells. In some embodiments, detecting intact (unlysed) target bacterial cells comprises staining the intact (unlysed) target bacterial cells with a marker that enables specific identification of intact (unlysed) target bacterial cells.

In some embodiments the methods further comprise providing a sample comprising the target bacteria; maintaining the sample in the absence of the candidate antimicrobial compound to provide a candidate antimicrobial compound-negative control target bacterial sample; exposing the candidate antimicrobial compound-negative control target bacterial sample to the cell-wall disruption condition; and determining the level of lysis and/or the level of remaining intact cells present in the candidate antimicrobial compound-negative control target bacterial sample. In some embodiments the methods further comprise comparing the level of lysis and/or the level of remaining intact cells present in the candidate antimicrobial compound-exposed target bacterial sample to the level of lysis and/or the level of remaining intact cells present in the candidate antimicrobial compound-negative target bacterial sample.

In some embodiments of the methods of this disclosure a plurality of concentrations of a candidate antimicrobial compound are assayed, either in parallel and/or in series. Accordingly, in some embodiments the methods comprise determining whether a target bacteria is susceptible to a candidate antimicrobial compound by a method comprising: providing a plurality of samples comprising the target bacteria; maintaining the plurality of samples in the presence of a plurality of concentrations of a candidate antimicrobial compound to provide a plurality of candidate antimicrobial compound-exposed target bacterial samples; exposing the plurality of candidate antimicrobial compound-exposed target bacterial samples to a cell-wall disruption condition; and determining whether the cell-wall disruption condition lyses target bacterial cells present in the plurality of antimicrobial compound-exposed target bacterial samples. Additionally, in some embodiments the methods comprise determining whether a target bacteria is susceptible to a candidate antimicrobial compound by a method comprising: providing a plurality of samples comprising the target bacteria; maintaining the plurality of samples in the presence of a plurality of concentrations of a candidate antimicrobial compound to provide a plurality of candidate antimicrobial compound-exposed target bacterial samples; exposing the plurality of candidate antimicrobial compound-exposed target bacterial samples to a cell-wall disruption condition; and determining the level of lysis and/or the level of remaining intact cells present in the candidate antimicrobial compound-exposed target bacterial sample; wherein the method is performed such that the level of lysis and/or remaining intact cells is determined without determining lysis or non-lysis on a cell-by-cell basis.

In some embodiments the plurality of concentrations of a In some embodiments the plurality of concentrations of a antimicrobial compound comprises a sample maintained antimicrobial compound comprises a sample maintained in the absence of the candidate antimicrobial compound. In some embodiments the methods further comprise determining the level of lysis and/or the level of remaining intact cells present in the plurality of candidate antimicrobial compound-exposed target bacterial samples. In some embodiments the methods further comprise comparing the level of lysis and/or the level of remaining intact cells present in the plurality of candidate antimicrobial compound-exposed target bacterial samples across the range of tested candidate antimicrobial compound concentrations. In some embodiments the methods further comprise determining the concentration of the candidate antimicrobial compound that causes lysis at or above a reference level of target bacterial cells present in the sample after exposing the sample to the cell-wall disruption condition. In some embodiments the methods further comprise determining the concentration of the candidate antimicrobial compound that causes lysis at or above a reference level of target bacterial cells present in the sample after exposing the sample to the cell-wall disruption condition.

In some embodiments of the methods of this disclosure a plurality of different densities of target bacterial cells are assayed, either in parallel and/or in series. Such embodiments may allow, for example, a determination of the effect of cell density on the antimicrobial activity of a tested compound. Accordingly, also provided are methods of determining whether a target bacteria is susceptible to a candidate antimicrobial compound, comprising: providing a plurality of samples comprising different densities of the target bacteria; maintaining the plurality of samples in the presence of a candidate antimicrobial compound to provide a plurality of candidate antimicrobial compound-exposed target bacterial samples; exposing the plurality of candidate antimicrobial compound-exposed target bacterial samples to a cell-wall disruption condition; and determining whether the cell-wall disruption condition lyses target bacterial cells present in the plurality of antimicrobial compound-exposed target bacterial samples. In some embodiments the methods further comprise determining the level of lysis of target bacterial cells present in the plurality of candidate antimicrobial compound-exposed target bacterial samples. In some embodiments the methods further comprise comparing the level of lysis of target bacterial cells present in the plurality of candidate antimicrobial compound-exposed target bacterial samples across the range of tested target bacterial cell densities. In some embodiments the methods further comprise determining the threshold density of target bacterial cells that is lysed in at least a threshold proportion after exposing the sample to the cell-wall disruption condition.

In some embodiments the time elapsed between the beginning of maintaining the sample in the presence of the candidate antimicrobial compound to the determination of whether the cell-wall disruption condition lyses target bacterial cells present in the candidate antimicrobial compound-exposed target bacterial sample is three hours of less.

This disclosure also provides kits for use in for determining whether a target bacteria is susceptible to an antimicrobial compound. The kits may comprise at least one component of a cell-wall disruption condition and/or a means for creating a cell-wall disruption condition; and a solid support for maintaining a sample comprising the target bacteria in the presence of the antimicrobial compound. In some embodiments the kits further comprise a solid support for exposing the antimicrobial compound-exposed target bacterial sample to a cell-wall disruption condition. In some embodiments the kits further comprise a detectable label that selectively labels intact cells or selectively labels lysed cells. In some embodiments the at least one component of a cell-wall disruption condition and/or a means for creating a cell-wall disruption condition comprises at least one detergent. In some embodiments the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80. In some embodiments the kits further comprise a container comprising the antimicrobial compound.

This disclosure also provides systems for determining whether a target bacteria is susceptible to an antimicrobial compound. The systems may comprise at least one component of a cell-wall disruption condition and/or a means for creating a cell-wall disruption condition; and a solid support for maintaining a sample comprising the target bacteria in the presence of the antimicrobial compound. In some embodiments the systems further comprise a solid support for exposing the antimicrobial compound-exposed target bacterial sample to the cell-wall disruption condition.

In some embodiments of the systems the target bacteria are not immobilized during the exposure to cell-wall disruption conditions. In some embodiments the systems further comprise determining the level of lysis and/or the level of remaining intact cells present in the antimicrobial compound-exposed target bacterial sample. In some embodiments the systems further comprise comparing the level of lysis and/or the level of remaining intact cells present in the antimicrobial compound-exposed target bacterial sample to a reference level to score the sample as sensitive or resistant to the at least one antimicrobial compound.

In some embodiments the systems further comprise a detectable label that selectively labels intact cells or selectively labels lysed cells. In some embodiments the at least one component of a cell-wall disruption condition and/or a means for creating a cell-wall disruption condition comprises at least one detergent. In some embodiments the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80. In some embodiments the systems further comprise a container comprising the antimicrobial compound.

In some embodiments the systems further comprise a positive control bacteria susceptible to the antimicrobial compound, wherein the positive control bacteria is lysed by a method comprising: providing a sample comprising the positive control bacteria; maintaining the sample in the presence of an antimicrobial compound to provide an antimicrobial compound-exposed positive control bacterial sample; and exposing the antimicrobial compound-exposed positive control bacterial sample to a cell-wall disruption condition.

In some embodiments the systems further comprise a negative control bacteria resistant to the antimicrobial compound, wherein the negative control bacteria is not lysed by a method comprising: providing a sample comprising the negative control bacteria; maintaining the sample in the presence of an antimicrobial compound to provide an antimicrobial compound-exposed negative control bacterial sample; and exposing the antimicrobial compound-exposed negative control bacterial sample to a cell-wall disruption condition.

In some embodiments the systems further comprise a work station for application of a cell wall disruption condition to the sample. In some embodiments the work station comprises a fluid dispenser for adding a cell wall disruption agent to the sample.

In some embodiments the systems further comprise a fluid dispenser for adding an antibiotic to the sample.

In some embodiments the systems further comprise a computer processor configured to control at least one of combining at least one anti-microbial compound with a sample, exposing the antimicrobial compound-exposed target bacterial sample to at least one cell lysis condition, and determining whether a cell-wall disruption condition lyses target bacterial cells present in an antimicrobial compound-exposed target bacterial sample.

In some embodiments of the methods, kits, and systems a single candidate compound or antimicrobial compound is added to a sample. In some embodiments of the methods, kits, and systems a mixture of at least two candidate compounds or antimicrobial compounds is substituted for the single compound. Unless clearly indicated otherwise by context, all of the methods, kits, and systems of this disclosure may be practiced by adding a single candidate compound or antimicrobial compound or by adding a mixture of at least two candidate compounds or antimicrobial compounds.

### BRIEF DESCRIPTION OF THE DRAWINGS

FIG. 1 shows the results of an antimicrobial compound susceptibility test using BacUni QuickFISH™ staining to differentiate between intact and lysed cells. The bottom panels were treated with meropenem and the top panels are negative controls not treated with meropenem. The left panels are E. coli strain BAA197 ESBL and the right panels are K. pneumoniae strain 3456. Both strains are susceptible to meropenem and that is reflected in the significant reduction in the number of stained cells in the bottom panels compared to the top panels.

FIG. 2 shows the results of an antimicrobial compound susceptibility test using BacUni QuickFISH™ staining to differentiate between intact and lysed cells. The bottom panels were treated with meropenem and the top panels are negative controls not treated with meropenem. The left panels are K. pneumoniae strain 13882 and the right panels are E. coli strain 23858. Both strains are susceptible to meropenem and that is reflected in the significant reduction in the number of stained cells in the bottom panels compared to the top panels.

FIG. 3 shows the results of an antimicrobial compound susceptibility test using BacUni QuickFISH™ staining to differentiate between intact and lysed cells. The bottom panels were treated with meropenem and the top panels are negative controls not treated with meropenem. The left panels are E. coli strain 25922 and the right panels are E. coli strain 35218. Both strains are susceptible to meropenem and that is reflected in the significant reduction in the number of stained cells in the bottom panels compared to the top panels.

FIG. 4 shows the results of an antimicrobial compound susceptibility test using BacUni QuickFISH™ staining to differentiate between intact and lysed cells. The bottom panel was treated with meropenem and the top panel is a negative control not treated with meropenem. The strain tested was K. oxytoca strain 43086. That strain is susceptible to meropenem and that is reflected in the significant reduction in the number of stained cells in the bottom panel compared to the top panel.

FIG. 5 shows the results of an antimicrobial compound susceptibility test using BacUni QuickFISH™ staining to differentiate between intact and lysed cells. The bottom panels were treated with meropenem and the top panels are negative controls not treated with meropenem. The left panels arc K. pneumoniae strain BAA1705 KPC+ and the right panels are K. pneumoniae strain BAA2146 NDM+. Both strains are resistant to meropenem and that is reflected in the similarity in the number of stained cells in the bottom panels (treated with meropenem) compared to the top panels (not treated with meropenem).

FIG. 6 shows the results of an antimicrobial compound susceptibility test using BacUni QuickFISH™ staining to differentiate between intact and lysed cells. The top panels are the meropenem sensitive K. pneumoniae strain 13882 and the bottom panels meropenem resistant K. pneumoniae strain BAA-2146 NDM+. As indicated in the figure, negative controls not treated with meropenem are compared to samples treated with 10 μg/ml, 20 μg/ml, or 40 μg/ml of meropenem.

FIG. 7 shows the results of an antimicrobial compound susceptibility test using BacUni QuickFISH™ staining to differentiate between intact and lysed cells. The tested strain was meropenem susceptible K. pneumoniae strain 13882. The left panels were treated with 10 μg/ml meropenem, while the right panels were not. The top panels were treated with cell wall disruption conditions comprising incubation in fixation buffer of 0.5% Triton×100, 100 mM Tris pH 9, 24% Ethanol, and 10 mM NaCl, while the bottom panels were not treated with fixation buffer. The results show that treatment with meropenem followed by exposure to fixation buffer results in the near complete absence of BacUni QuickFISH™ stained intact cells, indicating that cell lysis was extensive. In contrast, if either or both of meropenem treatment and fixation buffer exposure is omitted then stained cells are clearly present.

FIG. 8 shows the results of an antimicrobial compound susceptibility test using BacUni QuickFISH™ staining to differentiate between intact and lysed cells. The tested strain was meropenem resistant K. pneumoniae strain BAA2146 NDM+. The left panels were treated with 10 μg/ml meropenem, while the right panels were not. The top panels were treated with cell wall disruption conditions comprising incubation in fixation buffer of 0.5% Triton×100, 100 mM Tris pH 9, 24% Ethanol, and 10 mM NaCl, while the bottom panels were not treated with fixation buffer. The results show that treatment with meropenem followed by exposure to fixation buffer results in the near complete absence of BacUni QuickFISH™ stained intact cells, indicating that cell lysis was extensive. In contrast, if either or both of meropenem treatment and fixation buffer exposure is omitted then stained cells are clearly present.

FIG. 9 shows the results of an antimicrobial compound susceptibility test using BacUni QuickFISH™ staining to differentiate between intact and lysed cells. The tested strain was E. coli strain 35218. That strain is known to be susceptible to imipenem, ertapenem, and meropenem. The upper left panel is a control not treated with any antimicrobial compound. The other panels were treated with 10 μg/ml of imipenem, ertapenem, or meropenem, as indicated. The results show that the test is able to detect susceptibility of this strain to each antimicrobial compound.

FIG. 10 shows the results of an antimicrobial compound susceptibility test using BacUni QuickFISH™ staining to differentiate between intact and lysed cells. The tested strain was K. pneumoniae strain 13882. That strain is known to be susceptible to imipenem, ertapenem, and meropenem. The upper left panel is a control not treated with any antimicrobial compound. The other panels were treated with 10 μg/ml of imipenem, ertapenem, or meropenem, as indicated. The results show that the test is able to detect susceptibility of this strain to each antimicrobial compound.

FIG. 11 shows the results of an antimicrobial compound susceptibility test using BacUni QuickFISH™ staining to differentiate between intact and lysed cells. The tested strain was K. pneumoniae strain BAA2146 NDM+. That strain is known to be resistant to imipenem, ertapenem, and meropenem. The upper left panel is a control not treated with any antimicrobial compound. The other panels were treated with 10 μg/ml of imipenem, ertapenem, or meropenem, as indicated. The results show that the test is able to detect resistance of this strain to each antimicrobial compound.

FIG. 12 shows class A beta-lactamase Enterobacteriaceae. S=sensitive; I=intermediate; R=resistance; ND=not determined; % Δ=Percent change.

FIG. 13 shows class B beta-lactamase Enterobacteriaceae. S=sensitive; I=intermediate; R=resistance; ND=not determined; % Δ=Percent change; A+B=strain contains both Class A and Class B beta-lactamases.

FIG. 14 shows class C beta-lactamase Enterobacteriaceae. S=sensitive; I=intermediate; R=resistance; ND=not determined; % Δ=Percent change.

FIG. 15 shows class D beta-lactamase Enterobacteriaceae. S=sensitive; I=intermediate; R=resistance; ND=not determined; % Δ=Percent change.

FIG. 16 shows porin mutations in Enterobacteriaceae. S=sensitive; I=intermediate; R=resistance; ND=not determined; % Δ=Percent change; ΔOmp=porin mutation; A+C+P=strain contains both Class A and Class C beta-lactamases, along with a porin mutation; A+P=strain contains a Class A beta-lactamase, along with a porin mutation.

FIG. 17A shows other sample types. S=sensitive; I=intermediate; R=resistance; ND=not determined; % Δ=Percent change; A+B=strain contains both Class A and Class B beta-lactamases.

FIG. 17B shows other sample types. S=sensitive; I=intermediate; R=resistance; ND=not determined; % Δ=Percent change; A+B=strain contains both Class A and Class B beta-lactamases.

FIG. 18 shows class A beta-lactamase Enterobacteriaceae. S=sensitive; I=intermediate; R=resistance; % Δ=Percent change.

FIG. 19 shows class B beta-lactamase Enterobacteriaceae. S=sensitive; I=intermediate; R=resistance; % Δ=Percent change.

FIG. 20 shows class C beta-lactamase Enterobacteriaceae. S=sensitive; I=intermediate; R=resistance; % Δ=Percent change.

FIG. 21 shows class D beta-lactamase Enterobacteriaceae. S=sensitive; I=intermediate; R=resistance; % Δ=Percent change.

FIG. 22 shows porin mutations in Enterobacteriaceae. S=sensitive; I=intermediate; R=resistance, % Δ=Percent change; ΔOmp=porin mutation; A+C+P=strain contains both Class A and Class C beta-lactamases, along with a porin mutation; A+P=strain contains a Class A beta-lactamase, along with a porin mutation.

### DETAILED DESCRIPTION

### A. Introduction

The examples provided herein demonstrate that a cell-wall disruption condition may be applied to bacterial cells exposed to an antimicrobial compound to selectively lyse bacterial cells that are susceptible to the antimicrobial compound. Target bacterial cells not susceptible to the antimicrobial compound under the conditions of the method will not be selectively lysed by the cell-wall disruption condition used in the method. By controlling (1) the time of exposure to the antimicrobial compound, and/or (2) the concentration of cells and/or antimicrobial compound, and/or the intensity of the cell wall disruption condition, the method may be calibrated to distinguish subtle differences in bacterial susceptibility to a test antimicrobial compound. These observations have enabled the inventors to discover several new methods of assessing bacterial susceptibility and/or resistance to antimicrobial compounds; methods of treating subjects with a bacterial infection; systems for use in each type of method; and kits for use with each type of method, as well as other discoveries provided herein.

An important and particularly useful aspect of the methods of this invention is that they are based on directly assessing the susceptibility phenotype of bacteria in a sample. Therefore, the methods comprise assessing the antimicrobial compound susceptibility phenotype of bacteria in a sample directly and do not rely on detecting the presence or absence of a surrogate molecular marker that may correlate with susceptibility. Accordingly, it is an object of embodiments of this invention is to provide methods, systems, and kits for characterizing the antimicrobial compound susceptibility phenotype of bacteria in a sample.

An object of embodiments of the invention is delivery of actionable susceptibility scores in less than a day, preferably in four hours or less, and more preferably in two and a half hours or less after the identification of a primary patient sample with positive bacterial growth. This rapid delivery of test results from a phenotypic antimicrobial compound susceptibility test is a unique feature that distinguishes certain embodiments of the invention from the prior art.

An object of embodiments of the invention is evaluation of the susceptibility of bacteria in a sample to clinically relevant antibiotics in a timely manner and in an easy to understand format (either the bacteria is classified as sensitive and the antibiotic can be used, or resistant and other treatment alternatives should be sought).

By receiving fast, accurate, and simple-to-understand information, the clinician can provide the best therapeutic care. However, none of the “fast methods” currently commercially available or under development provide all these three points simultaneously. These methods rely on the molecular identification of the gene or the phenotypic identification of a beta-lactamase. The former, such as PCR or probe hybridization, does not usually discriminate between different levels of expression of the gene, and also fails to detect new or less common enzymes, or even variants of the common widespread enzymes. The latter comprises methodologies such as the one described in US Application Publication No. 2014/0080164 A1 which uses chromogenic beta-lactam substrates to identify the presence of the enzymes, or MALDI-TOF, which identifies the product of degradation of a beta-lactam. One of their limitations is that they may lack sensitivity to less efficient enzymes, such as class D beta-lactamases, and fail to provide any information about the susceptibility phenotype of bacteria in a sample. Of course, the susceptibility phenotype of bacteria in a sample is what matters in determining resistance or sensitivity to an antibiotic.

An object of embodiments of the invention is to provide an antimicrobial compound susceptibility test that is simple to use and does not require high levels of expertise for the reading and interpretation of the results. The use of a clear, objective cutoff point for the classification of an isolate as resistant or sensitive overcomes the need to look at the morphology of the cells, which can vary dramatically according to the antibiotic and concentrations used. Methods such as the one described in US Application Publication No. 20140206573 A1, not only do not present a clear cutoff for classification, but also require a considerable level of expertise in order to differentiate the multiple morphologies that the cells present after exposure to the antibiotic. Those and other features significantly limit the utility of the prior art methods. Embodiments of the invention do not suffer from such limitations and therefore provide an important improvement over prior art methods.

An object of embodiments of the invention is to provide an antibiotic susceptibility test having a reduced number of processing steps of the sample compared to at least one available alternative method. This confers consistency to the data generated by the method among other advantages. Some methods such as the one described in U.S. Pat. No. 8,785,148, require the covalent immobilization of living bacteria into a solid support, a step that requires technical skills in order to ensure reproducibility. The assay is also necessarily more time consuming. Other methods may require the previous preparation of the slides where the assay will be performed, as described in patent US Application Publication No. 2014/0206573 A1. Embodiments of the invention enable a method that starts in a liquid medium, reducing the necessary handling of the sample and in consequence the variability of the assay. The design of this technique allows starting the antimicrobial susceptibility testing (AST) immediately from a primary sample, such as blood, blood culture, bronchoalveolar lavage or urine, with a minimal processing of the sample. For example, in certain embodiments the sample is diluted in a buffer but the bacteria in the sample are not isolated. This feature is absent from other methods that have been described for fast ASTs and is a distinct advantage of some embodiments of the methods, systems, and kits of the invention.

It is also an object of the invention to provide a method that provides results comparable in accuracy to the results obtained by the gold standard method, the disk diffusion assay, no matter the complexity of the sample to be tested, while at the same time reducing the time-to-result by at least 5, 6, 7, 8, 9, 10, 11, 12, 13, 14, or 15 hours.

It is also an object of embodiments of the invention to provide methods, systems, and kits that provide consistent results independent of the beta-lactamase class present (it provides reliable data for classes A, B, C, and D), the number of different enzymes bacteria in a sample carry (bacteria in a sample with two different beta-lactamase classes have been successfully scored), and/or if there are additional non-specific mechanisms of resistance in the tested bacteria (such as porin deletions or down regulation). Several prior art methods are unable to provide one or more of these features.

The interpretation and use of the results given by the methods, systems, and kits of the invention is simple and straightforward, and similar to the actions taken when using the results from a disk diffusion assay (although adequate therapeutic measures can be taken in a much shorter timeframe when using the methods, systems, and kits of the invention, as compared to the disk diffusion assay). The classification of a strain as sensitive to a certain antimicrobial compound indicates that the use of that antimicrobial in the subject from where the sample originated is likely to be successful, leading to the cure of the patient. A classification of resistance to an antimicrobial indicates that its use is very unlikely to lead to a positive treatment outcome and that its use is not recommended. The ability to test a large panel of antimicrobials in rapidly and in parallel also presents advantages; by making the interpretative reading of the antibiogram, it will be possible to infer what mechanisms of resistance may be present in a sample and to select the most adequate treatment.

Unless otherwise defined herein, scientific and technical terms used in connection with the present disclosure shall have the meanings that are commonly understood by those of ordinary skill in the art. Before the present methods, systems, kits, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise. Further, unless otherwise required by context, singular terms shall include the plural and plural terms shall include the singular. The term “comprising” as used herein is synonymous with “including” or “containing”, and is inclusive or open-ended and does not exclude additional, unrecited members, elements or method steps.

### B. Target Bacteria

Essentially any bacteria can be assessed for antimicrobial compound susceptibility in the methods, systems, and kits disclosed herein. Particularly relevant bacteria include pathogenic bacteria that infect mammalian hosts (e.g., bovine, murine, equine, primate, feline, canine, and human hosts). In some embodiments, the target bacteria is selected from bacteria that infect and/or cause disease in a human host.

In some embodiments the target bacteria is a Gram-negative bacteria. Gram-negative bacteria are bacteria that do not retain crystal violet dye in the Gram staining protocol. In a Gram stain test, a counterstain (commonly safranin) is added after the crystal violet, coloring all gram-negative bacteria with a red or pink color. The counterstain is used to visualize the otherwise colorless gram-negative bacteria whose much thinner peptidoglycan layer does not retain crystal violet. The test itself is useful in classifying two distinct types of bacteria based on the structural differences of their bacterial cell walls. Gram-positive bacteria retain the crystal violet dye when washed in a decolorizing solution.

It is important to point out, though, that the Gram-positive and Gram-negative staining response is not a reliable phylogenetic character as these two kinds of bacteria do not form phylogenetically coherent groups. However, Gram-staining response of bacteria is an empirical criterion; its basis lies in the marked differences in the ultrastructure and chemical composition of two main kinds of prokaryotic cells that are found in nature. These two kinds of cells are distinguished from each other based upon the presence or absence of an outer lipid membrane, which is a reliable and fundamental characteristic of bacterial cells. All Gram-positive bacteria are bounded by only a single unit lipid membrane and they generally contain a thick layer (20-80 nm) of peptidoglycan responsible for retaining the Gram stain. A number of other bacteria that are bounded by a single membrane, but stain gram-negative due to either lack of the peptidoglycan layer or their inability to retain the Gram-stain because of their cell wall composition, also show close relationship to the gram-positive bacteria.

In some embodiments the target bacteria is a rod-shaped bacteria.

In some embodiments the target bacteria is a member of the family Enterobacteriaceae. The Enterobacteriaceae is a large family of Gram-negative bacteria that includes, along with many harmless symbionts, many of the more familiar pathogens, such as Salmonella, Escherichia coli, Yersinia pestis, Klebsiella and Shigella. Other disease-causing bacteria in this family include Proteus, Enterobacter, Serratia, and Citrobacter. This family is the only representative in the order Enterobacteriales.

In some embodiments the target bacteria is a Enterobacteriaceae that belongs to a genus selected from Alishewanella, Alterococcus, Aquamonas, Aranicola, Arsenophonus, Azotivirga, Blochmannia, Brenneria, Buchnera, Budvicia, Buttiauxella, Cedecea, Citrobacter, Cronobacter, Dickeya, Edwardsiella, Enterobacter, Erwinia, Escherichia, Ewingella, Grimontella, Hafnia, Hamiltonella, Klebsiella, Kluyvera, Leclercia, Leminorella, Moellerella, Morganella, Obesumbacterium, Pantoea, Pectobacterium, Phlomobacter, Photorhabdus, Plesiomonas, Pragia, Proteus, Providencia, Rahnella, Regiella, Raoultella, Salmonella, Samsonia, Serratia, Shigella, Sodalis, Tatumella, Trabulsiella, Wigglesworthia, Xenorhabdus, Yersinia, and Yokenella.

In some embodiments the target bacteria is a species selected from Klebsiella pneumoniae, Klebsiella oxytoca, Enterobacter aerogenes, Escherichia coli, Enterobacter cloacae and Proteus mirabilis.

In some embodiments the target bacteria is a non-fermenter bacteria. Non-fermenter bacteria are a taxonomic heterogene group of bacteria of the division Proteobacteria, which cannot catabolize glucose and therefore are not able to ferment. This does not exclude, automatically, that species can catabolize other sugars or have an anaerobiosis like fermenting bacteria. Exemplary non-limiting genera of non-fermenter bacteria include Acinetobacter, Bordetella, Burkholderia, Legionella, Moraxella, Pseudomonas, and Stenotrophomonas. Exemplary non-limiting species that are particularly pathogenic include Pseudomonas aeruginosa and Moraxella catarrhalis.

In some embodiments the target bacteria is a member of a genus selected from Bacteroides, Clostridium, Streptococcus, Staphylococcus, Pseudomonas, Haemophilus, Legionella, Mycobacterium, Escherichia, Salmonella, Shigella, Vibrio, and Listeria.

In some embodiments the target bacteria is selected from, Bacillus anthracia, Bordetella pertussis, Borrelia burgdorferi, Brucella aborus, Brucella canis, Brucella melitensis, Brucella suis, Campylobacter jejuni, Chlamydia pneumoniae, Chlamydia psittaci, Chlamydia trachomatis, Clostridium botulinum, Clostridium difficile, Clostridium perfringens, Clostridium tetani, Corynebacterium diphtheriae, Enterococcus faecalis, vancomycin-resistant Enterococcus faecalis, Enterococcus faecium, Escherichia coli, enterotoxigenic Escherichia coli (ETEC), enteropathogenic Escherichia coli, E. coli 0157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermis, Staphylococcus saprophyticus, methicillin-resistant Staphylococcus aureus (MRSA), vancomycin-resistant Staphylococcus aureus (VSA), Streptococcus agalactiae, Streptococcus pneumoniae, Streptococcus pyogenes, Treponema pallidum, Vibrio cholerae, and Yersinia pestis.

In some embodiments the target bacteria express at least one gene product that confers in whole or in part resistance to an antibiotic or class of antibiotics to the target bacteria. Examples of such gene products include beta-lactamases, porins and efflux pumps, and penicillin binding proteins. In some embodiments the methods, systems and kits of the invention are able to distinguish, phenotypically, between otherwise comparable bacteria that do and do not express the at least one gene product.

In some embodiments the target bacteria expresses at least one class A beta-lactamase. In some embodiments the target bacteria does not express at least one class A beta-lactamase. Class A beta-lactamase are serine-dependent beta-lactamases that can be found widespread in both Gram-negative and Gram-positive microorganisms. The functional feature that makes them different from the remaining classes of serine-dependent beta-lactamases is the mechanistic basis by which their active site, serine, is activated; in this class of enzymes, two amino acids, a glutamate and a lysine, are the catalytic residues which activate the serine and the catalytic water during acylation and dcacylation. Clinically relevant members of this group of enzymes are the KPC, NmcA, TEM, SHV or CTX—like enzymes; many of these enzymes are able to efficiently inactivate extended spectrum cephalosporins as well as carbapenems. These enzymes are inhibited by beta-lactamase inhibitors, such as clavulanic acid.

In some embodiments the target bacteria expresses at least one class B beta-lactamase. In some embodiments the target bacteria does not express at least one class B beta-lactamase. Class B enzymes are also called metallo-beta-lactamases and differ from the serine-dependent beta-lactamases in their catalytic mechanism. Class B enzymes are zinc-dependent beta-lactamases, and have one or two metal ions on their active site. All of them exhibit carbapenemase activity, and unlike serine-dependent enzymes, they are not inhibited by the classic beta-lactamase inhibitors. Some of the most common, and problematic, are the NDM, IMP and VIM-like enzymes which are widespread.

In some embodiments the target bacteria expresses at least one class C beta-lactamase. In some embodiments the target bacteria does not express at least one class C beta-lactamase. Class C beta-lactamases differ functionally from the remaining serine-dependent beta-lactamases on their catalytic residues, which comprise a lysine-tyrosine pair. These enzymes can be found both on the chromosome or in plasmids of Gram-negative microorganisms, and they represent an important clinical problem, since that not only can they hydrolyze penicillins efficiently, but also extended spectrum cephalosporins. Important enzymes belonging to this group are CMY, DHA, and MOX-like enzymes.

In some embodiments the target bacteria expresses at least one class D beta-lactamase. In some embodiments the target bacteria does not express at least one class D beta-lactamase. Class D beta-lactamases, also known as OXA enzymes due to their ability to hydrolyze oxacillin, are a heterogeneous group of enzymes that comprises narrow and extended spectrum beta-lactamases, as well as carbapenemases. Mechanistically, they rely on a post-translational modification of the active site lysine with a molecule of carbon dioxide, differing from the remaining serine-dependent beta-lactamases. Class D enzymes can be found in Gram-negative organisms, with clinical relevance in Enterobacteriaceae and non-fermenters such as Acinetobacter and Pseudomonas. Some important class D enzymes are OXA-48, -23 and -24 like enzymes.

Every bacterial cell possesses a considerable number of intrinsic porins and efflux systems. Porins are transmembrane proteins that act as pores, allowing the diffusion of different molecules from the extracellular space to the periplasm. Since the three dimensional structure of each of these porins is different, and based on factors such as charge and molecular size of the substrate, they have different specificities. The deletion of some of those proteins allows the prevention of the entrance of antimicrobial molecules into the periplasm (thereby decreasing susceptibility), without affecting the physiological functions of the cell. Efflux pumps in Gram-negative organisms are complex three-component systems that are able to expel molecules from inside the cell, either the periplasm or the cytoplasm, to the exterior of the cell. This removal of molecules, such as antibiotics, is an active process, which relies on an energy source such as ATP or proton gradient. While some efflux pumps tend to be more selective on the antibiotics they expel, many of them are able to efflux a large variety of substrates.

Porins and efflux systems have important metabolic functions and can also play a role in antibiotic resistance. The individual impact of decreases in porin concentration or increases in efflux pump concentration on the antimicrobial susceptibility is marginal, however; the levels of protection conferred by these changes allow the cells to accumulate mutations that may lead to increased resistance. Porins and efflux systems act synergistically with acquired mechanisms of resistance such as beta-lactamase enzymes, playing a role in the development of antibiotic resistance that cannot be underestimated.

In some embodiments the target bacteria does not express a particular porin protein and as a result has a reduced sensitivity to an antibiotic compound than it otherwise would if it expressed the at least one porin protein. In some embodiments the reduced sensitivity to the antibiotic is detected using a method of the invention.

In some embodiments the target bacteria does not express at least one particular efflux pump and as a result has an increased sensitivity to an antibiotic compound than it otherwise would if it did not express the same amount of the at least one particular efflux pump protein. In some embodiments the increased sensitivity to the antibiotic is detected using a method of the invention.

In some embodiments the target bacteria expresses at least one particular efflux pump and as a result has a reduced sensitivity to an antibiotic compound than it otherwise would if it expressed a higher amount of the at least one efflux pump protein. In some embodiments the reduced sensitivity to the antibiotic is detected using a method of the invention. In some embodiments the reduced sensitivity is scored as resistance.

Gram-positive and Gram-negative bacterial cell cytoplasmic membranes are surrounded by a peptidoglycan layer (thicker in Gram-positive) that is, amongst other functions, responsible for keeping the shape of the cell and to protect them from osmotic shock. This layer is composed of multiple cross-linked glycan strands. The formation, maintenance, and recycling of this layer is complex, and relies on multiple enzymes. A group of these enzymes is called penicillin binding proteins (PBP). More particularly PBP1a and PBP1b, PBP2 and PBP3 (E. coli numbering), have been the focus of the development of new beta-lactam antibiotics. PBP1a/b are the major transpeptidases-transglycosylases, and while the cell can cope with the loss of one of them, the simultaneous inhibition of both of them leads to cell lysis. Both PBP2 and PBP3 are transpeptidases, with the former being involved in the elongation of the cell, and the latter in the cell division and septation. Their inhibition leads to abnormal morphologies, which ultimately lead to cellular death and lysis. In some embodiments the target bacteria expresses at least one PBP. In some embodiments the target bacteria expresses at least one PBP selected from PBP1a and PBP1b, PBP2 and PBP3 or an equivalent in another type of bacteria. In some embodiments the target bacteria does not express at least one PBP. In some embodiments the target bacteria does not express at least one PBP selected from PBP1a and PBP1b, PBP2 and PBP3 or an equivalent in another type of bacteria.

### C. Sample Processing

The target bacteria are present in a sample to be tested by a method disclosed herein or using a kit or system disclosed herein. The sample may be a sample obtained from a subject. For example, the sample may be obtained from a subject who has a bacterial infection or who is suspected to have a bacterial infection or who is at risk of developing a bacterial infection. The sample may be maintained in the presence of an antimicrobial compound according to a method of this disclosure without first culturing the sample and/or without first isolating a particular type of bacteria in the sample. In some embodiments the sample is a sample from a culture of bacteria (which may be a culture of bacteria obtained from a subject). In some embodiments the culture is a log-phase culture. In some embodiments the culture is not a log-phase culture. In some embodiments the culture is a stationary-phase culture. In some embodiments the culture is a liquid culture. In some embodiments the culture is a solid-phase culture. In some embodiments the culture comprises only a single type of bacteria, such as a culture created by plating a mixture of bacterial cells and picking a single colony that grows up to establish the culture. In some embodiments the culture comprises a mixture of bacteria. For example, a primary sample from a subject may comprise a mixture of types of bacteria. Because the methods, systems, and kits of this invention enable directly assessing the susceptibility phenotype of bacteria in a sample, the methods, systems, and kits are particularly useful for characterizing the antimicrobial compound susceptibility of a mixture of bacteria.

A “subject sample” is a sample of biological material collected from a subject. A subject sample may be collected from a “sterile” body site such as blood, cerebral spinal fluid (CSF), abdominal fluid, pleural fluid, peritoneal fluid, joint fluid and pericardial fluid. Additional types of subject samples include urine, bronchoalveolar lavage (BAL) fluid, sputum, wound fluid, swab samples (such as wound swabs or genital swabs), stool, saliva, etc.

A “primary subject sample” or a “primary sample” is a subject sample that is collected from a subject and then processed using an antimicrobial compound susceptibility test (e.g., using a method, system, or kit of the invention) without diluting the subject sample by more than about 20×. Accordingly, a primary sample does not include blood collected and then diluted by 25× in liquid media or bacteria from a blood sample that were plated and grown on a solid media. A primary sample does include, e.g., blood collected and then diluted by 15× in culture media. In some embodiments the primary sample is an aliquot of an unprocessed subject sample. In some embodiments the primary sample is an aliquot of a subject sample that has been diluted by from 1× to about 20×, from 1× to 5×, from 5× to 10×, from 10× to 15×, or from 15× to about 20× in any liquid media suitable for maintaining the sample during the antimicrobial compound susceptibility test. In some embodiments the primary sample is an aliquot of a subject sample that has been diluted by 1×, 2×, 3×, 4×, 5×, 6×, 7×, 8×, 9×, 10×, 15, or about 20× in any liquid media suitable for maintaining the sample during the antimicrobial compound susceptibility test.

In some embodiments the methods, systems, and kits of this invention utilize a primary subject sample. In some embodiments the methods, systems, and kits of this invention utilize a non-primary subject sample.

Collection and Culture of Subject Samples

Blood culture is a commonly used diagnostic tool for suspected bloodstream infections. Blood cultures are prepared by extracting blood from the subject directly into prepared “blood culture bottles” which contain premeasured liquid media. Various types of liquid media are available; typically a set of two bottles are drawn together, one bottle designed to promote aerobic growth, the other to promote anaerobic growth. The large volume of a blood culture sample (10 mL) is required to ensure the sample contains some of the pathogen, which may be present at less than 10 colony forming units/mL (CFU/mL). After inoculation, the bottles are sent to the clinical microbiology lab, where they are placed into a blood culture machine The blood culture machine incubates at 35° C. while monitoring the bottles for growth through at least one of a variety of means, such as through a pH indicator dye. Growth may take several days to register, or may “go positive” in as few as 8 hours. Blood cultures are proven to have much higher sensitivity and faster detection of growth than cultures prepared directly onto solid media. Once detected, positive blood cultures are removed from the instrument, a Gram stain is performed, and the results are reported to the medical staff. The bottle may then be sub-cultured to isolate the pathogenic organism for identification and susceptibility testing. A sample may also be used for rapid analysis by molecular techniques.

Other types of subject samples may be collected and optionally cultured in a similar manner using methods known in the art.

Concentration/Enrichment

It is within the scope of the present invention to assess antibiotic susceptibility in any sample type, and at any concentration of microbial cells. In certain cases, where samples are derived from actively growing cultures, such as blood cultures, the concentration of bacteria may be sufficient to perform the assay directly, using simple detection methods, such as optical density to measure the presence of microorganisms and/or lysis. In other embodiments, samples may be processed to concentrate or enrich the microorganisms prior to use of the sample with a method, system or kit of the invention.

In some embodiments sample processing steps are included. For example, processing steps which selectively enrich the microorganisms in the sample through a variety of means may be used. For example, a sample collected on a swab may be enriched by releasing the cells in saline. Alternatively, the swab could be placed in a centrifuge and the cells removed by the physical force of centrifugation, or a combination of chemical and physical means could be used to concentrate the microorganisms.

Furthermore, the cells in a sample may be enriched by placing the sample under conditions which promote growth of the microorganisms contained within the sample, resulting in an increase in the concentration of microorganisms in the sample. Enrichment will often be achieved by application of the sample to liquid or solid growth media. The enrichment may be selective, such as a media which contains a chemical for instance an antibiotic which inhibits growth of sensitive strains; or be a growth factor which selectively promotes growth of certain strains, or the enrichment may operate generically, such as through promotion of aerobic or anaerobic respiration, and therefore generically promoting growth of certain strains. Alternatively, the media may be non-selective. The conditions which promote growth may be physical, such as heat, chemical such as nutrients, or a combination of physical and chemical conditions which promote growth. The cells in the sample may be selectively removed from a sample or sub-sample prior to being put under conditions which promote growth, for example by being passed through a size-exclusion filter. Alternatively, the entire sample may be placed under conditions which promote growth.

In some embodiments enrichment through promotion of growth is utilized to calibrate the sensitivity of the assay. For example, a sample may be placed under conditions which promote growth for a period of time sufficient to undergo approximately one doubling of selected microorganisms in the sample. The time period of one doubling will vary among selected microorganisms, or sample types, but can be determined empirically and extrapolated for general use under similar conditions. One doubling will be sufficient in many cases for the activity of selected antimicrobial compounds to be detectable by the assay. Alternatively, the sample may be allowed more time under the same conditions to allow two or more doublings of selected microorganism in the sample. As such, a sample of any type comprising any number of potential microorganisms may be placed under conditions which promote even a single cell to replicate enough times to produce detectable levels to be used in the methods, systems, and kits of the present invention. The enrichment method may be selectively sampled during the promotion of growth to determine if the density of organisms is sufficient to perform a test under selected parameters.

Therefore, it is within the scope of the invention to enrich subject sample by a variety of available means to provide a desired number of microorganisms to test. Examples of samples for which enrichment may in some embodiments be preferred include whole blood, cerebral spinal fluid, urine, bronchoalveolar lavage, swabs, saliva, etc. Also, enrichment may be used in some instances for certain samples which often do not require enrichment, such as blood culture or stool, or such as in mixed infections, or in cases where a large sample volume is not available.

Inoculum Preparation

The sample utilized in the methods, systems, and kits of the invention may be performed directly from an overnight culture or from a dilution into fresh broth followed by incubation.

An overnight culture is a culture that has incubated between 12-24 hours. This culture may be in a solid support (agar) or it may be in a liquid. Both cultures may have been obtained from a primary subject sample or from another culture, etc. The sample may be exposed to the at least one antimicrobial compound as it is, undiluted, or may be further diluted. This dilution may vary in extent; the final number of cells that can be used during the exposure to the antibiotic agent may range from 1×10 colony forming units (CFU) to 1×10¹⁰ CFU/mL. In some embodiments the number of cells exposed to the antibiotic agent is from 1×10 colony forming units (CFU) to 1×10¹⁰ CFU/mL, from 1×10 colony forming units (CFU) to 1×10² CFU/mL, from 1×10² colony forming units (CFU) to 1×10⁴ CFU/mL, from 1×10⁴ colony forming units (CFU) to 1×10⁶ CFU/mL, from 1×10⁶ colony forming units (CFU) to 1×10⁸ CFU/mL, or from 1×10⁸ colony forming units (CFU) to 1×10¹⁰ CFU/mL.

Another alternative is the dilution of the overnight culture into fresh broth and its growth for a certain duration of time before exposure to the antibiotic. This dilution can be performed into any culture broth that is able to sustain and allow the multiplication of a bacterial population. Some examples of adequate compositions are brain heart infusion, tryptic soy broth and Mueller Hinton broth. The extent of the dilution can vary. An overnight culture usually has between 1×10⁴ and 1×10¹⁰ CFU/mL; an acceptable dilution into fresh broth would be lead to a final amount of cells ranging from 1×10 to 1×10⁹CFU/mL.

The period of growth of the diluted cells can be variable and range from as low as 30 minutes or less to as long as six hours or more. The temperature of incubation will depend on the requirements of the bacteria species being tested.

### D. Antimicrobial Compounds

Without wishing to be bound by any theory, it is the present understanding of the inventors that the data reported herein in the examples (showing selective lysis of antimicrobial compound-susceptible bacterial cells) indicates that exposure of susceptible bacterial cells to an antimicrobial compound compromises the bacterial cell wall in a way that renders the compromised cell susceptible to lysis following treatment with cell lysis conditions, even in situations in which the cell lysis conditions are not sufficient to lyse bacterial cells that are resistant to the same antimicrobial compound. Accordingly, the methods, systems, and kits disclosed herein are broadly applicable to any antimicrobial compound that, directly or indirectly and by any mechanism, compromises the bacterial cell wall of bacterial cells that are susceptible to the antimicrobial compound but does not compromise the bacterial cell wall of bacterial cells that are susceptible to the antimicrobial compound.

Particularly relevant antimicrobial compounds include those used to treat subjects for bacterial infections. However, it is also contemplated herein that a candidate antimicrobial compound can also be tested for efficacy using methods, systems, and kits of this disclosure. Examples of the different classes of antimicrobial compounds that may be assessed using methods, systems, and kits of this disclosure include, but are not limited to, beta lactam antimicrobial compounds, beta lactamase inhibitors, aminoglycosides and aminocyclitols, quinolones, tetracyclines, macrolides, and lincosamides, as well as glycopeptides, lipopeptides and polypeptides, sulfonamides and trimethoprim, chloramphenicol, isoniazid, nitroimidazoles, rifampicins, nitrofurans, methenamine, and mupirocin.

In some embodiments, the antimicrobial compound is a cell wall biosynthesis inhibitor. An exemplary family of antimicrobial compounds that inhibit cell wall biosynthesis is the beta lactam antimicrobial compounds (e.g., penicillin derivatives, cephalosporins, monobactams, carbapenems, and (beta)-lactamase inhibitors). Some non-limiting examples of cell wall biosynthesis inhibitors are penicillin, ampicillin, benzathine penicillin, benzylpenicillin (penicillin G), phenoxymethylpenicillin (penicillin V), procaine penicillin, oxacillin, methicillin, nafcillin, cloxacillin, dicloxacillin, flucloxacillin, temocillin, amoxycillin, co-amoxiclav (amoxicillin+clavulanic acid), azlocillin, carbenicillin, ticarcillin, mezlocillin, piperacillin, aztreonam, bacitracin, cephalosporin, cephalexin, cefadroxil, cefalexin, cefprozil, cefdinir, cefdiel, cefditoren, cefoperazone, cefobid, cefotaxime, cefpodoxime, ceftazidime, ceftibuten, ceftizoxime, ceftriaxone, cefepime, ceftobiprole, cephalothin, cefazolin, cefaclor, cefuroxime, cefamandole, cefotetan, cefoxitin, ceftriaxone, carbapenem, imipenem, meropenem, ertapenem, faropenem, doripenem, aztreonam, clavulanic acid, tazobactam, sulbactam, vancomycin, teicoplanin, loracarbef, and ramoplanin.

In some embodiments the antimicrobial compound is selected form colistin, tigecycline, a cephalosporin, a carbapenem, cefoxitin, and fosfomycin.

In some embodiments the antimicrobial compound is a pharmaceutically acceptable derivative of an antimicrobial compound disclosed herein. As used herein, “pharmaceutically acceptable derivatives” of a an antimicrobial compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in the art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-mcthylglucamine, procaine, N-benzylphenethylaminc, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C≡C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C≡C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

In some embodiments the mechanism of action of the antimicrobial compound comprises inhibiting cell wall synthesis. Without wishing to be bound by any theory, it is a present understanding of the inventors that the mechanism of action of such antimicrobial compounds compromises the cell wall of susceptible bacterial cells and in turn causes susceptible cells to be more easily lysed by cell-lysis conditions that resistant cells.

It is also contemplated herein that susceptibility of target bacteria to antimicrobial compounds having another mechanism of action can be tested using the methods, systems, and kits described herein, even if the effect on the cell wall is indirect. Often changes in cellular processes are reflected by the state of the cell wall, which permits use of the methods, systems, and kits described herein even if the antimicrobial compound is not a direct cell wall synthesis inhibitor. It is well within the abilities of one of skill in the art to adapt the methods described herein such that the methods can be used with antimicrobial compounds having another mechanism of action. For example, target bacteria may be maintained in the presence of an antimicrobial compound that inhibits protein synthesis, RNA synthesis, and/or DNA synthesis, under conditions that lead to a compromising of cell wall integrity such that susceptible bacteria are selectively lysed by cell-wall disruption conditions.

In some embodiments of the methods, systems, and kits of the invention a combination of antimicrobial compounds is used. For example, a target (and/or control) bacteria may be exposed to the combination concurrently and/or sequentially. In some embodiments from 1 to 10 different antimicrobial compounds are used, from 1 to 5 different antimicrobial compounds are used, from 2 to 10 different antimicrobial compounds are used, from 2 to 5 different antimicrobial compounds are used, or from 5 to 10 different antimicrobial compounds are used. In some embodiments 1, 2, 3, 4, 5, 6, 7, 8, 9, 10, or more different antimicrobial compounds are used. In some embodiments utilizing a combination of antimicrobial compounds, the concentration of at least one antimicrobial compound in the combination is lower than the concentration of the at least one antimicrobial compound that would be used in an embodiment that uses only a single antimicrobial compound.

In some embodiments a candidate antimicrobial compound is substituted for an antimicrobial compound. Accordingly, the methods, kits, and systems described herein can be used to screen candidate antimicrobial compounds for efficacy against a bacterial sample, a bacterial strain or a mix of bacterial strains.

The methods, systems, and kits described herein may also be used to test at least two different concentrations/doses of an antimicrobial compound or candidate antimicrobial compound, determine efficacy of an antimicrobial compound and/or candidate antimicrobial compound, and/or to determine a minimum inhibitory concentration for an antimicrobial compound and/or candidate antimicrobial compound.

In some embodiments the antimicrobial compound or candidate antimicrobial compound is used at a concentration of from about 1 ng/ml to about 100 mg/ml, from about 10 ng/ml to about 10 mg/ml, from about 100 ng/ml to about 1 mg/ml, or from about 1 μg/ml to about 100 μg/ml. In some embodiments the antimicrobial compound or candidate antimicrobial compound is used at a concentration of from about 1 μg/ml to about 10 μg/ml, from about 5 μg/ml to about 15 μg/ml, from about 10 μg/ml to about 20 μg/ml, from about 15 μg/ml to about 25 μg/ml, from about 20 μg/ml to about 30 μg/ml, from about 25 μg/ml to about 35 μg/ml, from about 30 μg/ml to about 40 μg/ml, from about 35 μg/ml to about 45 μg/ml, from about 40 μg/ml to about 50 μg/ml, from about 50 μg/ml to about 60 μg/ml, from about 60 μg/ml to about 70 μg/ml, from about 70 μg/ml to about 80 μg/ml, from about 80 μg/ml to about 90 μg/ml, from about 90 μg/ml to about 100 μg/ml, from about 5 μg/ml to about 50 μg/ml, from about 10 μg/ml to about 50 μg/ml, from about 10 μg/ml to about 100 μg/ml, from about 20 μg/ml to about 100 μg/ml, or from about 10 μg/ml to about 40 μg/ml. In some embodiments the antimicrobial compound or candidate antimicrobial compound is used at a concentration of at least about 1 μg/ml, at least about 2 μg/ml, at least about 3 μg/ml, at least about 4 μg/ml, at least about 5 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 55 μg/ml, at least about 60 μg/ml, at least about 65 μg/ml, at least about 70 μg/ml, at least about 75 μg/ml, at least about 80 μg/ml, at least about 85 μg/ml, at least about 90 μg/ml, at least about 95 μg/ml, or at least about 100 μg/ml.

In some embodiments the antimicrobial compound or candidate antimicrobial compound is used at a concentration of about the minimum inhibitory concentration (MIC) of the antimicrobial compound in a growth inhibition assay. In some embodiments the antimicrobial compound or candidate antimicrobial compound is used at a concentration below the MIC of the antimicrobial compound in a growth inhibition assay. In some embodiments the antimicrobial compound or candidate antimicrobial compound is used at a concentration above the MIC of the antimicrobial compound in a growth inhibition assay.

Beta-lactam antibiotics target the penicillin binding proteins (PBP), and have a bactericidal activity, causing cellular death and lysis. Although all of them are able to induce cellular lysis, they differ in the time that mediates between exposure to the antibiotic and lysis. This observation is due to differences in their primary target. Those beta-lactams that essentially target PBP1a/b are responsible for a fast lysis while those that primarily target PBP2 or PBP3 initially induce morphological changes (formation of spheroplasts and filaments respectively) and only after which lysis occurs.

The differences in the lysis time pose a problem for those tests aiming at a fast identification of susceptibility. In some embodiments this issue is circumvented and a fast cell lysis is induced in the context of the methods, systems, and kits of this invention. As shown in the Examples, increasing the concentration of the cephalosporins to values several fold higher than the MIC values achieves this objective. With this increase, concentrations not only able to saturate all the PBP3 present in the cells, but also PBP1a/b, the main PBP responsible for a fast cellular lysis are achieved. The data that reported in the examples supports this increase in concentration. The ability to adjust the parameters of the assay in this manner is an advantage of the methods, systems, and kits of the invention compared to prior art methods.

In some embodiments the target bacteria are maintained in the presence of the antimicrobial compound for a period of time of from about 5 minutes to about 12 hours, from about 10 minutes to about 12 hours, from about 10 minutes to about 6 hours, from about 10 minutes to about 5 hours, from about 10 minutes to about 4 hours, from about 10 minutes to about 3 hours, from about 10 minutes to about 2 hours, from about 10 minutes to about 1 hour, from about 10 minutes to about 50 minutes, from about 10 minutes to about 40 minutes, from about 10 minutes to about 30 minutes, or from about 10 minutes to about 20 minutes, from about 20 minutes to about 6 hours, from about 20 minutes to about 5 hours, from about 20 minutes to about 4 hours, from about 20 minutes to about 3 hours, from about 20 minutes to about 2 hours, from about 20 minutes to about 1 hour, from about 20 minutes to about 50 minutes, from about 20 minutes to about 40 minutes, from about 20 minutes to about 30 minutes, from about 30 minutes to about 6 hours, from about 30 minutes to about 5 hours, from about 30 minutes to about 4 hours, from about 30 minutes to about 3 hours, from about 30 minutes to about 2 hours, from about 30 minutes to about 1 hour, from about 30 minutes to about 50 minutes, from about 30 minutes to about 40 minutes, or for about 30 minutes. In some embodiments the bacteria is maintained in the presence of the antimicrobial compound for no more than 6 hours, no more than 5 hours, no more than 4 hours, no more than 3 hours, no more than 2 hours, no more than 1 hour, no more than 50 minutes, no more than 40 minutes, no more than 30 minutes, no more than 20 minutes, or no more than 10 minutes. In some embodiments the bacteria is maintained in the presence of the antimicrobial compound for from 1 to 2 hours.

When determining antimicrobial susceptibilities, one of the factors that needs to be taken into consideration is the number of cells being tested. The extent to which each antimicrobial compound is affected varies, but when the number of cells being challenged with the antimicrobial compound increases, an increase in the MIC values is to be expected. A decrease in the MIC values is also expected when the number of cells exposed to the antimicrobial compound decreases. This effect is particularly important in the clinical environment and may explain some therapeutic failures reported in the art, since the number of cells present in some sites of infection may be higher than the one used for the traditional susceptibility tests. Again, because the present invention provides a phenotypic assay the ability to adjust the parameters of the assay to account for these issues is an advantage of the methods, systems, and kits of the invention compared to prior art methods.

### E. Methods of Lysis

As skilled artisans will appreciate, in view of this disclosure, any suitable cell-wall disruption conditions may be used in the methods, systems, and kits of the invention. Suitable cell-wall disruption conditions are conditions that cause a selective lyses of cells of susceptible bacteria treated with an antimicrobial compound. Exemplary cell-wall disruption conditions include conditions that comprise at least one of a detergent, a physical means of disrupting cells, alkaline conditions, a chemical cell-wall disruption agent, and an enzyme. In some embodiments cell wall-disruption conditions comprise at least two of a detergent, a physical means of disrupting cells, alkaline conditions, a chemical cell-wall disruption agent, and an enzyme.

In some embodiments cell wall-disruption conditions comprise at least one of a plurality of detergents, a plurality of physical means of disrupting cells, a plurality of alkaline conditions, a plurality of chemical cell-wall disruption agents, and a plurality of enzymes.

In some embodiments the cell-wall disruption condition comprises at least one detergent and at least one physical means of disrupting cells.

In some embodiments the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80. The detergent is generally used at a concentration of from about 0.01% to about 3%, such as from about 0.02% to about 3%, about 0.03% to about 3%, about 0.04% to about 3%, about 0.05% to about 3%, about 0.06% to about 3%, about 0.07% to about 3%, about 0.08% to about 3%, about 0.09% to about 3%, about 0.1% to about 3%, from about 0.2% to about 3%, from about 0.3% to about 3%, from about 0.4% to about 3%, from about 0.5% to about 3%, from about 0.6% to about 3%, from about 0.7% to about 3%, from about 0.8% to about 3%, from about 0.9% to about 3%, from about 1.0% to about 3%, from about 1.5% to about 3%, from about 2.0% to about 3%, from about 2.5% to about 3%, from about 0.01% to about 2.5%, from about 0.01% to about 2.0%, from about 0.01% to about 1.5%, from about 0.01% to about 1.0%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, from about 0.01% to about 0.2%, from about 0.01% to about 0.2%, from about 0.01% to about 0.2%, from about 0.01% to about 0.2%, from about 0.01% to about 0.2%, from about 0.01% to about 0.2%, from about 0.01% to about 0.2%, from about 0.01% to about 0.1%, from about 0.01% to about 0.09%, from about 0.01% to about 0.08%, from about 0.01% to about 0.07%, from about 0.01% to about 0.06%, from about 0.01% to about 0.05%, from about 0.01% to about 0.04%, from about 0.01% to about 0.03%, or from about 0.01% to about 0.02%. In some embodiments the concentration is about the value of one of the endpoints of one of the ranges listed in this paragraph. In some embodiments the concentration is at least about the value of one of the endpoints of one of the ranges listed in this paragraph. In some embodiments the concentration is no more than about the value of one of the endpoints of one of the ranges listed in this paragraph.

In some embodiments the cell wall disruption condition is applied for from about 1 second to 2 hours, such as from about 1 second to 5 seconds, from about 1 second to 10 seconds, from about 1 second to 15 seconds, from about 1 second to 20 seconds, from about 1 second to 25 seconds, from about 1 second to 30 seconds, from about 1 second to 35 seconds, from about 1 second to 40 seconds, from about 1 second to 45 seconds, from about 1 second to 50 seconds, from about 1 second to 55 seconds, from about 1 second to 1 minute, from about 30 seconds to 1 minute, from about 30 seconds to 2 minutes, from about 30 seconds to 5 minutes, from about 1 minute to 10 minutes, from about 5 minutes to 10 minutes, from about 10 minutes to 20 minutes, from about 20 minutes to 30 minutes, from about 30 minutes to 1 hour, or from about 1 hour to 2 hours.

In some embodiments the at least one physical means of disrupting cells comprises vortexing.

In some embodiments the physical means of disrupting cells comprises at least one of sonication and homogenization.

In some embodiments the alkaline conditions comprise a solution comprising NaOH.

In some embodiments the enzymatic conditions comprise exposure to an enzyme selected form lysozyme and lysostaphin.

In some embodiments the chemical cell disruption conditions comprise exposure to at least one of EDTA and lactic acid.

In some embodiments antimicrobial agent exposed and/or control cells are exposed to cell disruption conditions for a defined period of time.

### F. Detection of Differential Lysis

Skilled artisans will appreciate that the teachings of this disclosure can be broadly applied using any known or later developed method for detecting differential lysis of bacterial cells in a sample. Broadly speaking, and without limitation, such methods may be divided into 1) methods that comprise direct and/or indirect measurement of the presence and/or number of lysed cells; 2) methods that comprise the direct and/or indirect measurement of the presence of and/or the number of intact cells; and 3) methods that comprise direct and/or indirect measurement of the presence and/or number of lysed cells, and comprise the direct and/or indirect measurement of the presence of and/or the number of intact cells.

Methods of direct and/or indirect measurement of the presence and/or number of lysed cells include methods that comprise use of a marker to label at least one intracellular component present outside of a cell. Methods of direct and/or indirect measurement of the presence and/or number of intact cells include methods that comprise use of a marker to label at least one intracellular component present inside of a cell or to label at least one membrane, cell wall, or extracellular component present on the surface of an intact cell.

Examples of methods that comprise use of a marker to label at least one intracellular component present outside of a cell include methods of detecting/measuring protein released by a lysed cell (e.g., use of Coomassie Blue stain to detect/measure total protein concentration in a bacterial lysate), methods of detecting/measuring an enzyme released by a lysed cell (e.g., ATP luminescence measurement for the release of ATP from lysed bacterial cells), and methods of detecting/measuring nucleic acid released by a lysed cell (e.g., use of a peptide nucleic acid (PNA) probe with a fluorescent tag hybridized in lysate for measurement of nucleic acid release from bacterial cells).

Methods of direct and/or indirect measurement of the presence and/or number of intact cells and/or lysed cells also include methods that measure changes in a bacterial population. Such methods may be quantitative and/or qualitative. Examples of such methods include flow cytometry, OD₆₀₀ turbidity measurements, fluorescent in situ hybridization (FISH) using a probe common to cells of a particular type of bacteria (or to most or all of a class of bacteria, or to most or all bacteria), and bacterial stains such as toluidine blue stain to measure the presence or absence of bacterial cells. In some embodiments the sample is filtered after exposure to cell wall disruption conditions and before determining whether the cell-wall disruption conditions lyses target bacterial cells present in the antimicrobial compound-exposed target bacterial sample. Filtering may, for example, be by use of a 0.45 micron or 0.22 micron filter.

In some embodiments of the methods, systems, and kits of this invention, detection is performed without determining the number of bacterial cells that are lysed and/or that are intact. For example, certain prior art methods rely on identifying nucleoid material in a sample of immobilized target bacteria. Depending on the nucleoid morphology each analyzed cell is scored as lysed or intact. That is an example of a method comprising determining the number of bacterial cells that are lysed and/or that are intact. That is, the outcome of exposure to the antimicrobial compound conditions is determined on a cell-by-cell basis. Such methods are needlessly slow and laborious, among other drawbacks. In contrast, in most embodiments of the methods, systems, and kits of this invention, the method is performed such that the level of lysis and/or remaining intact cells is determined without determining lysis or non-lysis on a cell-by-cell basis. Additionally, in most embodiments of the methods, systems, and kits of this invention, the method is performed such that target bacterial cells are not immobilized prior to exposure to an antimicrobial compound. Moreover, in most embodiments of the methods, systems, and kits of this invention, the method is performed such that target bacterial cells are not immobilized prior to exposure to cell lysis conditions.

### G. Methods of Characterizing Bacterial Susceptibility to Antimicrobial Compounds

This disclosure provides methods of determining whether a target bacteria is susceptible to an antimicrobial compound. In some embodiments the methods comprise providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining whether the cell-wall disruption condition lyses target bacterial cells present in the antimicrobial compound-exposed target bacterial sample.

In some embodiments the antimicrobial compound-exposed target bacterial sample is exposed to a cell-wall disruption condition without immobilizing antimicrobial compound-exposed target bacteria. In some embodiments, doing the method in this way allows determining whether the cell-wall disruption condition lyses target bacterial cells present in the antimicrobial compound-exposed target bacterial sample using methods that would either not work or would be difficult to implement if the antimicrobial compound-exposed target bacterial sample is exposed to a cell-wall disruption condition after immobilizing the antimicrobial compound-exposed target bacteria. For example, flow cytometry may be used to determining whether the cell-wall disruption condition lyses target bacterial cells present in the antimicrobial compound-exposed target bacterial sample if the antimicrobial compound-exposed target bacterial sample is exposed to a cell-wall disruption condition without immobilizing antimicrobial compound-exposed target bacteria. However, flow cytometry cannot be used if instead the antimicrobial compound-exposed target bacterial sample is exposed to a cell-wall disruption condition after immobilizing the antimicrobial compound-exposed target bacteria.

If lyses of target bacterial cells present in the antimicrobial compound-exposed target bacterial sample is observed and/or if a loss of intact cells is observed, then this indicates that the target bacteria is susceptible to the antimicrobial compound. In some embodiments, a qualitative method is used to detect the presence of lysis and/or the loss of intact cells in order to determine whether a target bacteria is susceptible to an antimicrobial compound. In some embodiments, a quantitative method is used to detect the presence of lysis and/or the loss of intact cells in order to determine whether a target bacteria is susceptible to an antimicrobial compound.

If lysis of target bacterial cells present in the antimicrobial compound-exposed target bacterial sample is not observed and/or if persistence of intact cells is observed, then this indicates that the target bacteria is resistant to the antimicrobial compound. In some embodiments, a qualitative method is used to detect the absence of lysis and/or the persistence of intact cells in order to determine whether a target bacteria is susceptible to an antimicrobial compound. In some embodiments, a quantitative method is used to detect the absence of lysis and/or the persistence of intact cells in order to determine whether a target bacteria is susceptible to an antimicrobial compound.

In some embodiments the methods comprise performing a control assay using at least one of a positive control bacteria known to be susceptible to the antimicrobial compound and a negative control bacteria known to be resistant to the antimicrobial compound.

For example, in some embodiments the methods comprise determining whether the cell-wall disruption condition lyses target bacterial cells present in the antimicrobial compound-exposed target bacterial sample by a method comprising: A) providing a positive control sample comprising a positive control bacteria known to be sensitive to the antimicrobial compound; maintaining the positive control sample in the presence of the antimicrobial compound to provide an antimicrobial compound-exposed positive control bacterial sample; exposing the antimicrobial compound-exposed positive control bacterial sample to a cell-wall disruption condition; and determining the level of lysis of positive control bacterial cells present in the antimicrobial compound-exposed target bacterial sample; and B) comparing the level of lysis of target bacterial cells present in the antimicrobial compound-exposed target bacterial sample to the level of lysis of positive control bacterial cells present in the antimicrobial compound-exposed positive-control bacterial sample.

In some embodiments the methods comprise determining whether the cell-wall disruption condition lyses target bacterial cells present in the antimicrobial compound-exposed target bacterial sample by a method comprising: A) providing a negative control sample comprising a negative control bacteria known to be resistant to the antimicrobial compound; maintaining the negative control sample in the presence of the antimicrobial compound to provide an antimicrobial compound-exposed negative control bacterial sample; exposing the antimicrobial compound-exposed negative-control bacterial sample to a cell-wall disruption condition; and determining the level of lysis of the negative control bacterial cells present in the antimicrobial compound-exposed negative-control bacterial sample; and B) comparing the level of lysis of target bacterial cells present in the antimicrobial compound-exposed target bacterial sample to the level of lysis of negative control bacterial cells present in the antimicrobial compound-exposed negative-control bacterial sample.

In some embodiments the methods comprise determining whether the cell-wall disruption condition lyses target bacterial cells present in the antimicrobial compound-exposed target bacterial sample by a method comprising: A) providing a positive control sample comprising a positive control bacteria known to be sensitive to the antimicrobial compound; maintaining the positive control sample in the presence of the antimicrobial compound to provide an antimicrobial compound-exposed positive control bacterial sample; exposing the antimicrobial compound-exposed positive control bacterial sample to a cell-wall disruption condition; and determining the level of lysis of positive control bacterial cells present in the antimicrobial compound-exposed target bacterial sample; B) providing a negative control sample comprising a negative control bacteria known to be resistant to the antimicrobial compound; maintaining the negative control sample in the presence of the antimicrobial compound to provide an antimicrobial compound-exposed negative control bacterial sample; exposing the antimicrobial compound-exposed negative-control bacterial sample to a cell-wall disruption condition; and determining the level of lysis of the negative control bacterial cells present in the antimicrobial compound-exposed negative-control bacterial sample; and C) comparing the level of lysis of target bacterial cells present in the antimicrobial compound-exposed target bacterial sample to the level of lysis of positive control bacterial cells present in the antimicrobial compound-exposed positive-control bacterial sample and to the level of lysis of negative control bacterial cells present in the antimicrobial compound-exposed negative-control bacterial sample. In some embodiments the methods comprise providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining whether the cell-wall disruption condition lyses target bacterial cells present in the antimicrobial compound-exposed target bacterial sample. In some embodiments the antimicrobial compound-exposed target bacterial sample is exposed to a cell-wall disruption condition without immobilizing antimicrobial compound-exposed target bacteria.

In some embodiments the methods comprise providing a sample comprising the target bacteria; maintaining the sample in the presence of an antimicrobial compound to provide an antimicrobial compound-exposed target bacterial sample; exposing the antimicrobial compound-exposed target bacterial sample to a cell-wall disruption condition; and determining the level of lysis and/or the level of remaining intact cells present in the antimicrobial compound-exposed target bacterial sample cells present in the antimicrobial compound-exposed target bacterial sample; wherein the method is performed such that the level of lysis and/or remaining intact cells is determined without determining lysis or non-lysis on a cell-by-cell basis.

In some embodiments the methods further comprise comparing the level of lysis and/or the level of remaining intact cells present in the antimicrobial compound-exposed target bacterial sample to a reference level to score the sample as sensitive or resistant to the at least one antimicrobial compound.

In some embodiments if the level of lysis present in the antimicrobial compound-exposed target bacterial sample is at or above a reference level and/or the level of remaining intact cells present in the antimicrobial compound-exposed target bacterial sample is at or below a reference level, the target bacteria are scored as sensitive to the antimicrobial compound if the cell-wall disruption condition lyses target bacterial cells present in the antimicrobial compound-exposed target bacterial sample, the target bacteria is susceptible to the at least one antimicrobial compound.

In some embodiments if the level of lysis present in the antimicrobial compound-exposed target bacterial sample is not at or above a reference level and/or the level of remaining intact cells present in the antimicrobial compound-exposed target bacterial sample is not at or below a reference level, the target bacteria are scored as resistant to the antimicrobial compound if the cell-wall disruption condition does not lyse target bacterial cells present in the antimicrobial compound-exposed target bacterial sample, the target bacteria is not susceptible to the at least one antimicrobial compound.

In some embodiments the target bacteria are not immobilized during the exposure to cell-wall disruption conditions.

In some embodiments the methods do not comprise detecting the presence or absence of at least one target bacteria protein and/or at least one target bacteria nucleic acid. In some embodiments the sample comprising the target bacteria is a primary sample. In some embodiments the sample comprising the target bacteria is an in vitro cultured sample.

In some embodiments the in vitro cultured sample is provided by obtaining a sample comprising the target bacteria from a subject and culturing target bacteria in the subject sample to provide the in vitro cultured sample.

In some embodiments the target bacteria is Gram-negative. In some embodiments the target bacteria is rod-shaped. In some embodiments the target bacteria is a member of the family Enterobacteriaceae. In some embodiments the target bacteria is a non-fermenter bacteria.

In some embodiments the antimicrobial compound is a bactericidal antimicrobial compound. In some embodiments the antimicrobial compound comprises a β-lactam ring. In some embodiments the antimicrobial compound is a carbapenem. In some embodiments the antimicrobial compound is selected from colistin or a derivative thereof, tigecycline or a derivative thereof, a cephalosporin or a derivative thereof, a carbapenem or a derivative thereof, cefoxitin or a derivative thereof, and fosfomycin or a derivative thereof.

In some embodiments the sample is maintained in the presence of a concentration of the at least one antimicrobial compound that is at least the minimum inhibitory concentration of the at least one antimicrobial compound. In some embodiments the sample is maintained in the presence of the antimicrobial compound for about two hours or less.

In some embodiments the cell-wall disruption condition comprises at least one of a detergent, a physical means of disrupting cells, alkaline conditions, a chemical cell-wall disruption agent, and an enzyme. In some embodiments the cell-wall disruption condition comprises a detergent and a physical means of disrupting cells. In some embodiments the detergent is selected from at least one of Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

In some embodiments, if the cell-wall disruption condition lyses target bacterial cells present in the antimicrobial compound-exposed target bacterial sample, the target bacteria is susceptible to the antimicrobial compound. In some embodiments, if the cell-wall disruption condition does not lyse target bacterial cells present in the antimicrobial compound-exposed target bacterial sample, the target bacteria is not susceptible to the antimicrobial compound. In some embodiments the methods further comprise determining the extent of lysis of target bacterial cells present in the antimicrobial compound-exposed target bacterial sample.

In some embodiments determining whether the cell-wall disruption condition lyses target bacterial cells present in the antimicrobial compound-exposed target bacterial sample does not comprise counting target bacterial cells.

In some embodiments, determining whether the cell-wall disruption condition lyses target bacterial cells present in the antimicrobial compound-exposed target bacterial sample comprises detecting intact (unlysed) target bacterial cells. In some embodiments, determining whether the cell-wall disruption condition lyses target bacterial cells present in the antimicrobial compound-exposed target bacterial sample comprises detecting lysed target bacterial cells. In some embodiments, determining whether the cell-wall disruption condition lyses target bacterial cells present in the antimicrobial compound-exposed target bacterial sample comprises detecting intact (unlysed) target bacterial cells and detecting lysed target bacterial cells. In some embodiments, determining whether the cell-wall disruption condition lyses target bacterial cells present in the antimicrobial compound-exposed target bacterial sample comprises detecting intact (unlysed) target bacterial cells and does not comprise detecting lysed target bacterial cells. In some embodiments, determining whether the cell-wall disruption condition lyses target bacterial cells present in the antimicrobial compound-exposed target bacterial sample comprises detecting lysed target bacterial cells and does not comprise detecting intact (unlysed) target bacterial cells. In some embodiments, detecting intact (unlysed) target bacterial cells comprises counting the intact (unlysed) target bacterial cells. In some embodiments, detecting intact (unlysed) target bacterial cells comprises staining the intact (unlysed) target bacterial cells with a marker that enables specific identification of intact (unlysed) target bacterial cells.

In some embodiments the methods further comprise providing a sample comprising the target bacteria; maintaining the sample in the absence of the antimicrobial compound to provide an antimicrobial compound-negative control target bacterial sample; exposing the antimicrobial compound-negative control target bacterial sample to the cell-wall disruption condition; and determining the level of lysis and/or the level of remaining intact cells present in the antimicrobial compound-negative control target bacterial sample. In some embodiments the methods further comprise comparing the level of lysis and/or the level of remaining intact cells present in the antimicrobial compound-exposed target bacterial sample to the level of lysis and/or the level of remaining intact cells present in the antimicrobial compound-negative target bacterial sample. In some embodiments of the methods of this disclosure a plurality of concentrations of an antimicrobial compound are assayed, either in parallel and/or in series. Accordingly, in some embodiments the methods comprise determining whether a target bacteria is susceptible to an antimicrobial compound by a method comprising: providing a plurality of samples comprising the target bacteria; maintaining the plurality of samples in the presence of a plurality of concentrations of an antimicrobial compound to provide a plurality of antimicrobial compound-exposed target bacterial samples; exposing the plurality of antimicrobial compound-exposed target bacterial samples to a cell-wall disruption condition; and determining whether the cell-wall disruption condition lyses target bacterial cells present in the plurality of antimicrobial compound-exposed target bacterial samples. Additionally, in some embodiments the methods comprise determining whether a target bacteria is susceptible to an antimicrobial compound by a method comprising: providing a plurality of samples comprising the target bacteria; maintaining the plurality of samples in the presence of a plurality of concentrations of an antimicrobial compound to provide a plurality of antimicrobial compound-exposed target bacterial samples; exposing the plurality of antimicrobial compound-exposed target bacterial samples to a cell-wall disruption condition; and determining the level of lysis and/or the level of remaining intact cells present in the antimicrobial compound-exposed target bacterial sample; wherein the method is performed such that the level of lysis and/or remaining intact cells is determined without determining lysis or non-lysis on a cell-by-cell basis.

In some embodiments the plurality of concentrations of an antimicrobial compound comprises a sample maintained in the absence of the antimicrobial compound. In some embodiments the methods further comprise determining the level of lysis and/or the level of remaining intact cells present in the plurality of antimicrobial compound-exposed target bacterial samples. In some embodiments the methods further comprise comparing the level of lysis and/or the level of remaining intact cells present in the plurality of antimicrobial compound-exposed target bacterial samples across the range of tested antimicrobial compound concentrations. In some embodiments the methods further comprise determining the concentration of the antimicrobial compound that causes lysis at or above a reference level of target bacterial cells present in the sample after exposing the sample to the cell-wall disruption condition. In some embodiments the methods further comprise determining the concentration of the antimicrobial compound that causes lysis at or above a reference level of target bacterial cells present in the sample after exposing the sample to the cell-wall disruption condition.

In some embodiments of the methods of this disclosure a plurality of different densities of target bacterial cells are assayed, either in parallel and/or in series. Such embodiments may allow, for example, a determination of the effect of cell density on the antimicrobial activity of a tested compound. Accordingly, also provided are methods of determining whether a target bacteria is susceptible to an antimicrobial compound, comprising: providing a plurality of samples comprising different densities of the target bacteria; maintaining the plurality of samples in the presence of an antimicrobial compound to provide a plurality of antimicrobial compound-exposed target bacterial samples; exposing the plurality of antimicrobial compound-exposed target bacterial samples to a cell-wall disruption condition; and determining whether the cell-wall disruption condition lyses target bacterial cells present in the plurality of antimicrobial compound-exposed target bacterial samples. In some embodiments the methods further comprise determining the level of lysis of target bacterial cells present in the plurality of antimicrobial compound-exposed target bacterial samples. In some embodiments the methods further comprise comparing the level of lysis of target bacterial cells present in the plurality of antimicrobial compound-exposed target bacterial samples across the range of tested target bacterial cell densities. In some embodiments the methods further comprise determining the threshold density of target bacterial cells that is lysed in at least a threshold proportion after exposing the sample to the cell-wall disruption condition.

In some embodiments the time elapsed between the beginning of maintaining the sample in the presence of the antimicrobial compound to the determination of whether the cell-wall disruption condition lyses target bacterial cells present in the antimicrobial compound-exposed target bacterial sample is three hours of less.

In some embodiments of the methods the period of time from initiation of maintaining the sample in the presence of an antimicrobial compound to provide an antimicrobial compound-exposed target bacterial sample to determining whether the cell-wall disruption condition lyses target bacterial cells present in the antimicrobial compound-exposed target bacterial sample is from about 5 minutes to about 12 hours, from about 10 minutes to about 12 hours, from about 10 minutes to about 6 hours, from about 10 minutes to about 5 hours, from about 10 minutes to about 4 hours, from about 10 minutes to about 3 hours, from about 10 minutes to about 2 hours, from about 10 minutes to about 1 hour, from about 10 minutes to about 50 minutes, from about 10 minutes to about 40 minutes, from about 10 minutes to about 30 minutes, or from about 10 minutes to about 20 minutes, from about 20 minutes to about 6 hours, from about 20 minutes to about 5 hours, from about 20 minutes to about 4 hours, from about 20 minutes to about 3 hours, from about 20 minutes to about 2 hours, from about 20 minutes to about 1 hour, from about 20 minutes to about 50 minutes, from about 20 minutes to about 40 minutes, from about 20 minutes to about 30 minutes, from about 30 minutes to about 6 hours, from about 30 minutes to about 5 hours, from about 30 minutes to about 4 hours, from about 30 minutes to about 3 hours, from about 30 minutes to about 2 hours, from about 30 minutes to about 1 hour, from about 30 minutes to about 50 minutes, from about 30 minutes to about 40 minutes, or for about 30 minutes. In some embodiments of the methods the period of time from initiation of maintaining the sample in the presence of an antimicrobial compound to provide an antimicrobial compound-exposed target bacterial sample to determining whether the cell-wall disruption condition lyses target bacterial cells present in the antimicrobial compound-exposed target bacterial sample is no more than 6 hours, no more than 5 hours, no more than 4 hours, no more than 3 hours, no more than 2 hours, no more than 1 hour, no more than 50 minutes, no more than 40 minutes, no more than 30 minutes, no more than 20 minutes, or no more than 10 minutes.

It is also an object of the invention to provide a method that provides results comparable in accuracy to the results obtained by the gold standard method, the disk diffusion assay, no matter the complexity of the sample to be tested, while at the same time reducing the time-to-result by at least 5, 6, 7, 8, 9, 10, 11, 12, 13, 14, or 15 hours.

In some embodiments the time elapsed between the beginning of maintaining the sample in the presence of the antimicrobial compound to the determination of whether the cell-wall disruption condition lyses target bacterial cells present in the antimicrobial compound-exposed target bacterial sample is 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hour or less. In some embodiments the time elapsed between the beginning of maintaining the sample in the presence of the antimicrobial compound to the determination of whether the cell-wall disruption condition lyses target bacterial cells present in the antimicrobial compound-exposed target bacterial sample is from 30 minutes to 6 hours, from 1 hour to 6 hours, from 2 hours to 6 hours, from 3 hours to 6 hours, from 30 minutes to 3 hours, from 1 hour to 3 hours, or from 2 hours to 3 hours.

The methods, kits, and systems provided herein may be implemented in a “high throughput” format for determining resistance/susceptibility from at least two samples simultaneously, iteratively, concurrently, or consecutively. In some embodiments the number of samples assayed simultaneously is in the range of from 1 to 10000 samples; in some embodiments the following ranges of sample number may be assayed in the high throughput implementation: from 1 to 5000, from 1 to 2500, from 1 to 1250, from 1 to 1000, from 1 to 500, from 1 to 250, from 1 to 100, from 1 to 50, from 1 to 25, from 1 to 10, from 1 to 5, from 7500 to 10000, from 5000 to 10000, from 4000 to 10000, from 3000 to 10000, from 2000 to 10000, from 1000 to 10000, from 500 to 10000, from 100 to 1000, from 200 to 1000, from 300 to 1000, from 400 to 1000, or from 500 to 1000. The term “high-throughput” encompasses automation of the methods described herein using e.g., robotic pipettors, robotic samplers, robotic shakers, data processing and control software, liquid handling devices, incubators, detectors, hand-held detectors etc. For the purposes of automation, the number of samples tested at one time may correspond to the number of wells in a standard plate (e.g. E-well plate, 12-well plate, 96-well plate, 384-well plate, etc.). The samples can be obtained from a plurality of individuals, or from a plurality of samples obtained from a single individual, or both. A high-throughput system permits testing susceptibility of a bacterial strain to multiple antimicrobial compounds simultaneously, to test for susceptibility to a particular antimicrobial compound in a plurality of samples, and/or to test multiple doses of the same antimicrobial compound in a sample. As used herein the phrase “panel of at least two different antimicrobial compounds” refers to a plurality of different antimicrobial compound compounds assessed at approximately the some time.

Setting Thresholds for Sensitivity and Resistance

The method, systems, and kits of the invention utilize the ability to detect cell lysis as a way to measure the effect of an antimicrobial compound on a microorganism, where relatively higher amounts of lysis are interpreted to indicate susceptibility to the drug being tested. The threshold or cut-off level of lysis which indicates resistance or sensitivity have been developed through empirical testing of characterized strains with known levels of resistance (measured using standard phenotypic methods). Threshold levels will vary by species, bacterial concentration, drug, drug concentration, detection method, etc. It is within the scope of this invention to adjust threshold levels according to these, and other parameters. Skilled artisans may utilize the teachings of this disclosure to identify appropriate cut-offs for any given type of target bacteria in any type of sample for any type of antimicrobial compound.

### H. Lack of Correlation Between Gene and Phenotype Can Occur

A common assumption is that the presence of genes encoding specific beta-lactamases in bacteria in a sample will translate into a phenotype of resistance. This assumption has been the basis for the development of innumerous detection methods, and has been used in the clinical setting in decision-making in the field of infectious diseases. However, this is not a correct assumption, as there are many factors that influence how the presence of a gene translates into the effects it will have on the susceptibility phenotype of the bacteria. Being aware of these factors and understanding the importance and advantages of phenotypic assays (the gold standard for antibiotic sensitivity is a phenotypic assay), will have a deep contribution to the outcome of antibiotic therapy, and all the social and economic factors associated with it.

A factor to take into consideration is the amount of enzyme produced, which is known to dramatically impact the susceptibility phenotype. The acquisition of new promoters, or the mutation of the ones that are already present, can lead to increases/decreases in the copy number of the transcripts, which will ultimately lead to an increase/decrease in the amount of enzyme that is present in the cell. It has also been described in the literature that resistance genes present in some bacteria are not expressed, either due to the lack of a promoter or due to deleterious mutations in the promoter. The amount of enzyme produced also varies from species to species; while some enzymes show little to no effect on the susceptibility phenotype when expressed in one species, the effect can be significantly different when present in other species. These considerations confound the use of assays based on detection of the presence or absence of a gene or gene product in bacteria in a sample. While the disk diffusion assay avoids some of these issues, that assay presents other concerns that can confound the reliability and/or usability of assay results. For example, the disk diffusion assay usually requires significantly more time to complete. It also requires culturing the bacteria for longer which can lead to changes in phenotype so that the outcome of the assay is not representative of the bacteria in the subject.

Another frequently underestimated factor is the method of detection that is used. Many methods that detect specific enzymes not only detect that specific enzyme but also its mutant variants (their genes can differ by as little as a single base pair), and often fail to distinguish between them. These mutations are most of the times evolution driven and are responsible for changes in the substrate profile, which means that they become better at inactivating the antibiotic, but many times, they also become capable of inactivating new molecules. An example of an increase substrate profile is the one seen with the TEM-family. TEM-1, one of the first enzymes to be described, was a good penicillinase, and was also capable of hydrolyzing early generation cephalosporins, lacking the ability to use later generation cephalosporins as a good substrate. However, point mutations quickly allowed it to expand that spectrum, with many derivatives becoming resistant to later generation cephalosporins and also to beta-lactamase inhibitors. A more dramatic change in the spectrum of activity is the one see with OXA-163, a derivative of OXA-48. OXA-48 has a good activity against carbapenems, but it is sensitive to the action of the later generation cephalosporin, ceftazidime. However, strains with OXA-163 become resistant to ceftazidime, while losing the resistance to carbapenems. Examples like the ones we describe, all regarding clinically common enzymes, may lead the clinician to use a drug to which the bacteria has become resistant, while abstaining from using one drug to which the bacteria is, or has become, sensitive, with obvious negative implications.

Additionally, the presence of concomitant non-specific mechanisms of resistance, which are usually not included/detected in the current detection methods, such as porin or up-regulation of efflux systems. While their synergistic effect is barely noticeable in catalytic efficient enzymes, it is important when they are present simultaneously with less efficient enzymes. A classic example is the widespread OXA-48 beta-lactamase, which when present in strains lacking these mechanisms is unable to confer a phenotype of resistance to the beta-lactams, but becomes responsible for a phenotype of resistance when associated with them.

Ultimately, the phenotypic behavior of a specific isolate depends on the combination of innumerous individual and interrelated factors, which cannot be exclusively measured by a qualitative measure such as the presence of specific resistance determinants, such as particular beta-lactamase genes. While these detection methods are useful, the phenotypic methods of the invention that directly evaluate the behavior of a cell in the presence of clinically relevant antibiotics will have several advantages.

### I. Methods of Treatment

This disclosure also provides methods of treating a bacterial infection in a subject that comprise determining that a target bacteria is susceptible to an antimicrobial compound. For example, in some embodiments the methods comprise A) determining that a target bacteria is susceptible to an antimicrobial compound by a method comprising: providing a subject sample comprising target bacteria; maintaining the subject sample comprising target bacteria in the presence of an antimicrobial compound to provide an antimicrobial compound-exposed subject bacterial sample; exposing the antimicrobial compound-exposed subject sample to a cell-wall disruption condition; and determining that the cell-wall disruption condition lyses target bacterial cells present in the antimicrobial compound-exposed subject bacterial sample; and B) administering a therapeutically effective amount of the antimicrobial compound to the subject to thereby treat the bacterial infection in the subject. The determining that a target bacteria is susceptible to an antimicrobial compound may be performed using any method provided herein.

In some embodiments the methods comprise determining that a target bacteria is susceptible to an antimicrobial compound before the antimicrobial compound is first administered to the subject to treat the bacterial infection. In some embodiments the methods comprise determining that a target bacteria is susceptible to an antimicrobial compound after the antimicrobial compound is administered to the subject to treat the bacterial infection. For example, determining that a target bacteria is susceptible to an antimicrobial compound may be performed in order to ensure that the target bacteria is not acquiring resistance to the antimicrobial compound after initiation of treatment of the bacterial infection by the antimicrobial compound.

### J. Systems

This disclosure also provides systems for use in determining whether a target bacteria is susceptible to an antimicrobial compound. The system may be localized or dispersed. In some embodiments the system is located on a table or in a cabinet. In some embodiments the system is located within a room. In some embodiments the system is located within a single building. In some embodiments the system is geographically dispursed to multiple sites of up to hundreds or thousands of miles apart. The various components of the system are used together to perform a process or are manufactured or acquired for that purpose.

The systems generally comprise at least one component of a cell-wall disruption condition and/or a means for creating a cell-wall disruption condition; and a solid support for maintaining a sample comprising the target bacteria in the presence of the antimicrobial compound. In some embodiments the systems further comprise a solid support for exposing the antimicrobial compound-exposed target bacterial sample to a cell-wall disruption condition. The solid support for maintaining a sample comprising the target bacteria in the presence of the antimicrobial compound and the solid support for exposing the antimicrobial compound-exposed target bacterial sample to a cell-wall disruption condition may be the same or different. For example, a single Eppendorf tube may be used or a series of Eppendorf tubes may be used. Alternatively, a single substrate may comprise both solid supports in different locations.

In some embodiments the systems further comprise a detectable label that selectively labels intact cells or selectively labels lysed cells. In some embodiments the kits comprise at least one detectable label that selectively labels intact cells and at least one detectable label that selectively labels lysed cells.

In some embodiments, the at least one component of a cell-wall disruption condition and/or a means for creating a cell-wall disruption condition comprises at least one detergent. For example, a tube may be included that comprises the detergent. The detergent may be provided as a concentrated stock solution that is diluted when exposing the antimicrobial compound-exposed target bacterial sample to a cell-wall disruption condition. In some embodiments the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

In some embodiments the system further comprises an antimicrobial compound.

In some embodiments the system further comprises a sample comprising a target bacteria.

In some embodiments the system further comprises a positive control bacteria susceptible to the antimicrobial compound.

In some embodiments the system further comprises a negative control bacteria susceptible to the antimicrobial compound.

In some embodiments the systems and methods of this disclosure are implemented using an automated assay system. For example, the system may be an automatic specimen analyzing system. The system may comprise assay trays. After the operator loads the specimen trays into the system, at least one of various operations including incubation after inoculation, adding reagents and analysis of the specimen following incubation may be handled automatically without further operator involvement. A computer-type processor may be used to control the system so that the various operations are carried out in appropriate sequence and the results of the analysis are recorded with specific reference to the sample analyzed.

Typically, the specimens are arranged in a plurality of specimen trays wherein each of the trays is adapted to contain a plurality of specimens. The system may include one or more tray towers for supporting a plurality of the specimen trays. A work station may be located adjacent to the tray tower for selectively treating and analyzing the specimens. Selectively operable tray moving devices associated with the work station may be arranged to remove the tray from the tray tower and move it to the work station or to reinsert the tray in the tray tower after the operations at the work station have been completed.

The systems generally include a fluid dispensing work station within a housing as well. The system may include a work station having a source of fluid that is to be added to the specimen during processing. The work station may include a fluid dispensing area and a nozzle for dispensing the fluid. In some embodiments the fluid comprises a cell wall disruption agent. In some embodiments the fluid comprises at least one antimicrobial compound.

Multimodal carrier mechanisms may also be included. For example, the carrier mechanism may operate in a first mode for movement in the work station during fluid dispensing operations. For example, during dispensing of at least one fluid comprising a cell wall disruption agent and/or at least one fluid comprising at least one antimicrobial compound. The carrier mechanism may also operate in a second mode for movement outside the work station to do another processing function not involving the work station. A controller mechanism may selectively switch the mode of operation of the carrier mechanism between modes.

The system may further include a docking mechanism that couples the nozzle to the carrier when it operates in its first mode to help dispense fluid. The docking mechanism may release the nozzle from the carrier when it operates in its second mode, freeing the carrier to do other processing functions out of association with the nozzle.

The system may optionally include a second work station for performing a second processing function on the specimen. Additional work stations may be provided when and as needed. For example, in some embodiments a first work station is configured for adding an antimicrobial compound to the sample and a second work station is configured for adding a cell wall disruption agent to the sample.

The system may also comprise a mechanism for controlling the temperature of the sample while the sample is maintained in the presence of the antimicrobial agent and/or while the sample is exposed to cell-wall disruption conditions. In some embodiments the system comprises a mechanism for maintaining the target bacteria in suspension while the sample is maintained in the presence of the antimicrobial agent and/or while the sample is exposed of cell-wall disruption conditions.

### K. Kits

This disclosure also provides kits for use in determining whether a target bacteria is susceptible to an antimicrobial compound. In general the kits comprise a container or package comprising the other components of the kit. The kits generally further comprise at least one component of a cell-wall disruption condition and/or a means for creating a cell-wall disruption condition; and a solid support for maintaining a sample comprising the target bacteria in the presence of the antimicrobial compound. In some embodiments the kits further comprise a solid support for exposing the antimicrobial compound-exposed target bacterial sample to a cell-wall disruption condition. The solid support for maintaining a sample comprising the target bacteria in the presence of the antimicrobial compound and the solid support for exposing the antimicrobial compound-exposed target bacterial sample to a cell-wall disruption condition may be the same or different. For example, a single Eppendorf tube may be used or a series of Eppendorf tubes may be used. Alternatively, a single substrate may comprise both solid supports in different locations.

In some embodiments the kits further comprise a detectable label that selectively labels intact cells or selectively labels lysed cells. In some embodiments the kits comprise at least one detectable label that selectively labels intact cells and at least one detectable label that selectively labels lysed cells.

In some embodiments, the at least one component of a cell-wall disruption condition and/or a means for creating a cell-wall disruption condition comprises at least one detergent. For example, a tube may be included that comprises the detergent. The detergent may be provided as a concentrated stock solution that is diluted when exposing the antimicrobial compound-exposed target bacterial sample to a cell-wall disruption condition. In some embodiments the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

In some embodiments the kit further comprises an antimicrobial compound. The antimicrobial compound may be provided as part of a kit designed to specifically assess antimicrobial compound resistance to the antimicrobial compound or as part of a positive and/or negative control.

### EXAMPLES

### Example 1

### Selective Lysis of Susceptible Bacteria Exposed to Meropenem

An isolated colony from an overnight Tryptocase Soy Agar (TSA) plate of each bacterial strain was suspended in 1 ml Tryptocasc Soy Broth (TSB) in a 5 ml BD polystyrene round bottom tube. TSB cultures were incubated shaking at 37° C. overnight. Ten of the overnight culture was inoculated into lml TSB and incubated shaking at 37° C. for three hours. Two 2500 aliquots of each log-phase culture were then transferred to two 2 ml Eppendorf microcentrifuge tubes. One tube contained 5000 of Normal Saline (BD); the second tube contained 5000 Normal Saline with meropenem at 10 μg/ml (final concentration of meropenem of 6.67 10 μg/ml). Tubes were inverted and then incubated at 37° C. stagnant for thirty minutes. Then 2500 of lysis buffer (0.5% SDS in PSB) (final concentration of SDS of 0.125%) was added to each tube and the tubes were vortexed for 5 seconds. Post incubation, all tubes were spun at 10000 G for 5 minutes. Supernatant was decanted and the pellet resuspended in Normal Saline. Tubes were vortexed again. OD₆₀₀ was measured using Eppendorf uvettes in a spectrophotometer. The delta between the OD₆₀₀ of the normal saline control and the OD₆₀₀ for the test sample exposed to meropenem in normal saline was used to determine susceptibility of the bacterial strain to meropenem.

The results of performing the method on strains previously considered susceptible to meropenem are shown in Table 1. Species abbreviations: sp. Klebsiella pneumoniae (K. pneumo), Klebsiella oxytoca (K. oxy), Enterobacter aerogenes (E. aero), Escherichia coli (E. coli), and Enterobacter cloacae (E. clo).

These strains represent the most common species of Enterobacteriaceae isolated from human samples, including some with beta-lactam resistance.

TABLE 1

| | | Control | | | |
|---|---|---|---|---|---|
| Species | Strain | OD₆₀₀ | Mero OD₆₀₀ | % Change | R or S |
| E. coli ESBL | 790 | 0.954 | 0.126 | 87% | S |
| E. aero | 13048 | 2.255 | 0.077 | 97% | S |
| K. pneumo | 9633 | 2.03 | 0.015 | 99% | S |
| K. pneumo | 8308 | 2.19 | 0.053 | 98% | S |
| K. pneumo | 33495 | 2.09 | 0.097 | 95% | S |
| E. coli | JM109 | 0.642 | 0.079 | 88% | S |
| E. clo | 13047 | 1.295 | 0.06 | 95% | S |
| E. coli | 23848 | 0.68 | 0.021 | 97% | S |
| E. coli ESBL | Baa197 | 0.897 | 0.236 | 74% | S |
| K. oxy | 43086 | 1.442 | 0.02 | 99% | S |
| E. coli | 25922 | 1.853 | 0.064 | 97% | S |
| E. coli | 35218 | 0.963 | 0.1 | 90% | S |
| E. coli | 51422 | 1.537 | 0.031 | 98% | S |
| K. pneumo | 700603 | 1.139 | 0.065 | 94% | S |

For 13 of 14 strains the OD₆₀₀ measurement of turbidity decreased by at least 88% in the sample treated with meropenem in comparison to the negative control that was not treated with the antimicrobial compound. Here turbidity is proportional to the number of intact cells. This indicates that application of cell-wall disruption conditions (here treatment with 0.125% SDS and vortexing for 5 seconds) caused lysis of cells that had been treated with meropenem at a rate 88% higher than the rate of lysis of cells that had not been treated with meropenem. A single strain previously considered sensitive to the antimicrobial compound (E. coli Baa197) had a turbidity decrease of only 74%. This strain is known to produce an extended-spectrum beta-lactamase enzyme, which may have some activity on carbapenem, therefore rendering the strain partially resistant in this assay.

The results of performing the method on strains previously considered resistant to meropenem are shown in Table 2. The tested resistant strains were Klebsiella pneumoniae carbapenemase (KPC)-producing bacteria (abbreviated “KPC”), KPC-producing Klebsiella oxytoca (abbreviated “KPC K. oxy), and Klebsiella pneumoniae producing Metallo-beta-lactamase-1 (NDM-1) (abbreviated “NDM”).

TABLE 2

| | Resistance | | Control | Mero | % | R or |
|---|---|---|---|---|---|---|
| Species | Mechanism | Strain | OD₆₀₀ | OD₆₀₀ | Change | S |
| K. pneumo | KPC | 41217 | 1.856 | 1.764 | 5% | R |
| K. pneumo | KPC | 24605 | 2.165 | 2.0363 | 6% | R |
| K. pneumo | KPC | KPC12 | 1.88 | 1.844 | 2% | R |
| K. pneumo | KPC | 4121 | 1.686 | 1.734 | −3% | R |
| K. oxy | KPC | 46532 | 0.894 | 0.259 | 71% | S |
| K. pneumo | KPC | 65707 | 2.142 | 1.887 | 12% | R |
| K. pneumo | KPC | 12213 | 1.38 | 1.454 | −5% | R |
| K. pneumo | NDM | 2146 | 1.914 | 1.576 | 18% | R |
| K. pneumo | KPC | 1705 | 1.246 | 1.65 | −32% | R |

For 8 of the 9 strains the OD₆₀₀ measurement of turbidity decreased by 18% or less in the sample treated with meropenem in comparison to the negative control that was not treated with the antimicrobial compound. (In some cases the % change value is negative, indicating that the measured turbidity value was higher in the sample treated with the antimicrobial compound. That result represents variability in the measurements and is properly scored as a positive test result for susceptibility to the antimicrobial compound.) That result indicates the absence or near absence of meropenem-induced cell lysis in the cells of the known resistant strains tested. At such high concentrations of antimicrobial compound, even resistant organisms may experience a slight weakening of the outer membrane of the bacteria until the carbapenemase enzyme is produced at a concentration to effectively hydrolyze the antimicrobial compound, as not all organisms produce the enzyme at the same rate.

A single strain considered resistant to the antimicrobial compound (K. oxy 46532-KPC) had a turbidity decrease of 71%. This qualifies the strain as sensitive. This difference between the result of this assay and prior analysis of this strain may be a consequence of the carbapenemase concentration produced by this strain or a species other than K. pneumoniae with carbapenem-resistance. This strain may produce a relatively lower amount of the carbapenemase enzyme, which leads to a higher concentration of carbapenem in the assay solution, and thus to a more significant weakening of the membrane. This may be remedied by trying a variety of concentrations of carbapenem or time of exposure to effectively differentiate this strain as resistant. If the difference is a mechanism of the different species, which may mean some slight difference in the physical components of the outer membrane, then a different concentration of the detergent in the lysis buffer may help resolve this as a resistant strain. The bulk of the data presented in this example indicates that for the conditions tested a percentage change of greater than about 85% indicates a strain is susceptible.

### Example 2

### Selective Lysis of Bacteria Exposed to Meropenem and Cefotaxime

In this experiment the following strains were tested: strain K. pneumoniae 13882 (previously considered meropenem sensitive and cefotaxime sensitive); E. coli BAA-197 (ESBL) (which expresses an extended-spectrum (beta)-lactamase enzyme and was previously considered meropenem sensitive and cefotaxime resistant); K. pneumoniae BAA-1705 (KPC) (which expresses a carbapenemase enzyme and was previously considered meropenem resistant and cefotaxime resistant); and K. pneumoniae BAA-2146 (NDM1) (which expresses the metallo-beta-lactamase-1 enzyme and was previously considered meropenem resistant and cefotaxime resistant).

An isolated colony from an overnight Tryptocase Soy Agar (TSA) plate of each bacteria was suspended in 1 ml Tryptocase Soy Broth (TSB) in a 5 ml BD polystyrene round bottom tube. TSB cultures were incubated at 37° C. for 1.5 hours shaking. Three 250 μl aliquots of each stationary-phase culture were then transferred to three 2 ml Eppendorf microcentrifuge tubes. One tube contained 500 μl of Normal Saline (BD); the second tube contained 500 μl Normal Saline with meropenem at 10 μg/ml; and the third tube contained 500 μl Normal Saline with cefotaxime (abbreviated eel) at 10 μg/ml. Tubes were inverted then incubated at 37° C. stagnant for thirty minutes. Then 2500 of lysis buffer (0.5% SDS in PSB) was added to each tube and each tube was vortexed for 5 seconds. Post incubation, all tubes were spun at 10000 G for 5 minutes. Supernatant was decanted and the pellet resuspended in Normal Saline. Tubes were vortexed. OD₆₀₀ was measured using Eppendorf uvettes in a spectrophotometer. The delta between the control and the meropenem normal saline, or the control and the cefotaxime normal saline was used to determine susceptibility.

TABLE 3

| Strain | Control | Mero | Cef | % diff Mero | % diff Cef |
|---|---|---|---|---|---|
| K. pneumo | 0.364 | 0.127 | 0.11 | 65.1 | 69.8 |
| 13882 | | | | | |
| E. coli BAA- | 0.361 | 0.046 | 0.292 | 87.3 | 19.1 |
| 197 (ESBL) | | | | | |
| K. pneumo | 0.507 | 0.423 | 0.334 | 16.6 | 34.1 |
| BAA-1705 | | | | | |
| (KPC) | | | | | |
| K. pneumo | 0.3 | 0.301 | 0.28 | −0.3 | 6.7 |
| BAA-2146 | | | | | |
| (NDM1) | | | | | |

The results indicate that the assay assigned susceptibility and resistance to the strains consistent with prior work. Namely, the assay scored the strains as follows: strain K. pneumoniae 13882 (meropenem sensitive and cefotaxime sensitive); E. coli BAA-197 (ESBL) (meropenem sensitive and cefotaxime resistant); K. pneumoniae BAA-1705 (KPC) (meropenem resistant and cefotaxime resistant); and K. pneumoniae BAA-2146 (NDM1) (meropenem resistant and cefotaxime resistant). In this experiment the data indicate that for the conditions tested a percentage change of at least about 65% indicates a strain is susceptible.

Interestingly, this data shows that the differential cell lysis assay may be used to assess antimicrobial compound resistance based on different molecular mechanisms, ESBL (E. coli BAA-197) and Carbapenems (BAA1705 and BAA2146). The difference in the growth phase may contribute to a decreased susceptibility of the cell outer membrane to the action of the antimicrobial compound or lysis.

### Example 3

### Selective Lysis of Bacteria Exposed to Meropenem

An alternative to the spectrophotometer-based measurement of changes in turbidity for measuring cell lysis following treatment with the cell lysis conditions in Examples 1 and 2 is to stain samples using a stain that distinguishes between intact cells and lysed cells. In this example staining with BacUni QuickFISH™ was used to identify intact (i.e., non-lysed) cells. BacUN1 is a universal bacteria PNA probe that binds to an rRNA sequence present in most Gram-positive and Gram-negative bacteria. The PNA probe binds to universal rRNA in intact bacterial cells and the entire cell will appears green with fluorescent microscopy. If the bacterial cell had been lysed then the rRNA target would have been released from the bacterial cell and even of somewhat labeled it would not appear as a fluorescent bacterial cell.

An isolated colony from an overnight Tryptocase Soy Agar (TSA) plate of each bacteria was suspended in 1 ml Tryptocase Soy Broth (TSB) in a 5 ml BD polystyrene round bottom tube. TSB cultures were incubated at 37° C. overnight. Ten μl of overnight culture was inoculated into 1 ml TSB and incubated shaking at 37° C. for three hours. Two 250 μl aliquots of each log-phase culture was transferred to two 2 ml Eppendorf microcentrifuge tubes. One tube contained 500 μl of Normal Saline (BD); the second tube contained 500 μl Normal Saline with meropenem at 10 μg/ml. Tubes were inverted and then incubated at 37° C. stagnant for thirty minutes. Then 2500 lysis buffer (0.5% SDS in PSB) was added to each tube and the tubes were vortexed for 5 seconds. Post incubation, the entire culture was filtered through a 1 uM polycarbonate 18 mm filter. The filter was transferred to a plain glass slide and placed on a 55° C. heat block. Then 2 drops of 100% methanol were added to adhere the filter to the slide. Then 30 μl of BacUni QuickFISH™ hybridization buffer was added to the slide and a 50×22 mm coverslip was applied. The slide was then incubated at 55° C. for 15 minutes. The slide was viewed on fluorescence microscope using 60× oil immersion on Dual TxR/FITC. Images were taken with a 1 second exposure.

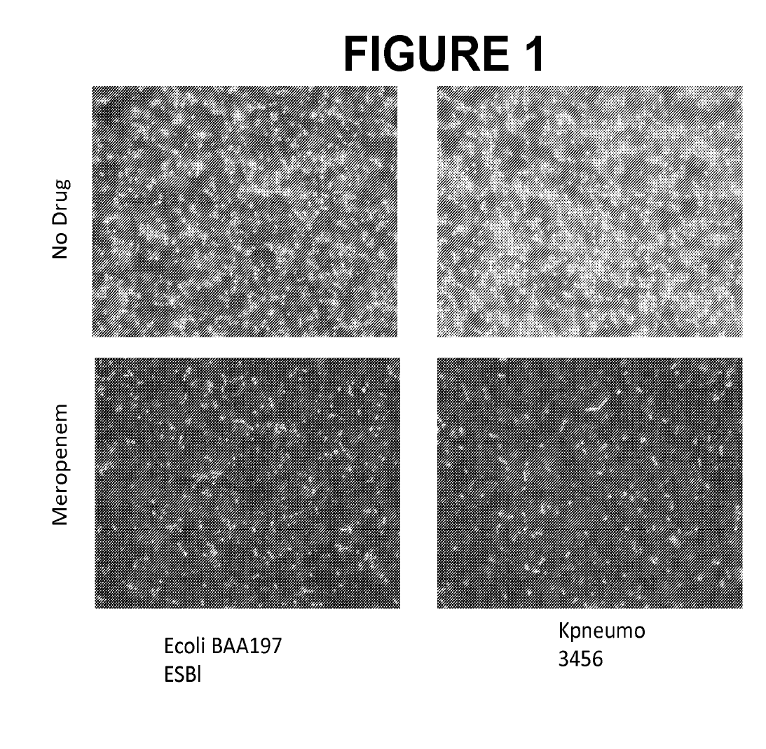

FIG. 1 shows the results of an antimicrobial compound susceptibility test using BacUni QuickFISH™ staining to differentiate between intact and lysed cells. The bottom panels were treated with meropenem and the top panels are negative controls not treated with meropenem. The left panels are E. coli strain BAA-197 ESβL and the right panels are K. pneumoniae strain 3456. Both strains are susceptible to meropenem and that is reflected in the significant reduction in the number of stained cells in the bottom panels compared to the top panels.

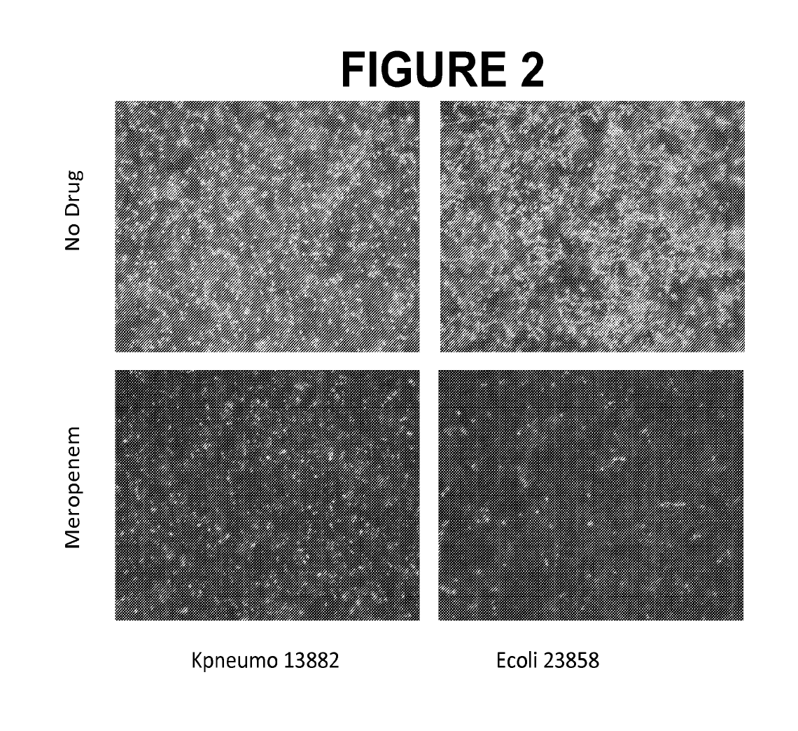

FIG. 2 shows the results of an antimicrobial compound susceptibility test using BacUni QuickFISH™ staining to differentiate between intact and lysed cells. The bottom panels were treated with meropenem and the top panels are negative controls not treated with meropenem. The left panels are K. pneumoniae strain 13882 and the right panels are E. coli strain 23858. Both strains are susceptible to meropenem and that is reflected in the significant reduction in the number of stained cells in the bottom panels compared to the top panels.

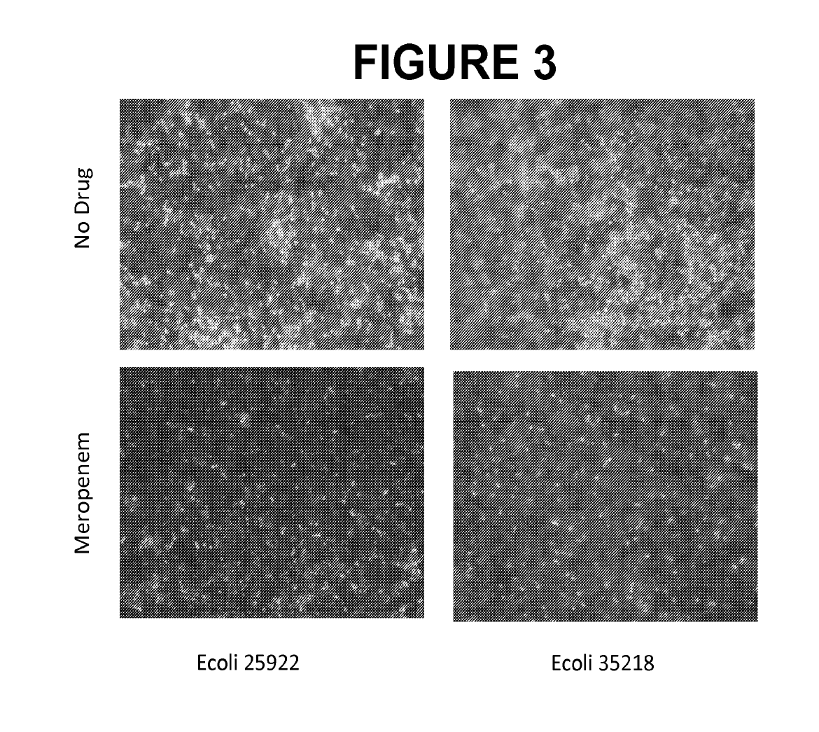

FIG. 3 shows the results of an antimicrobial compound susceptibility test using BacUni QuickFISH™ staining to differentiate between intact and lysed cells. The bottom panels were treated with meropenem and the top panels are negative controls not treated with meropenem. The left panels are E. coli strain 25922 and the right panels are E. coli strain 35218. Both strains are susceptible to meropenem and that is reflected in the significant reduction in the number of stained cells in the bottom panels compared to the top panels.

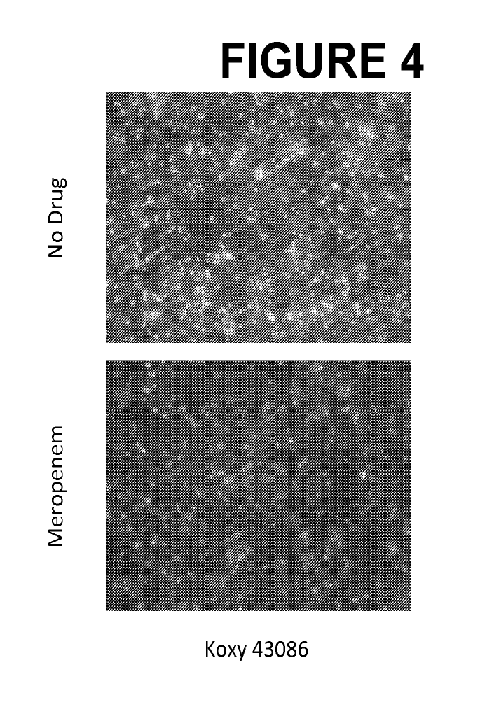

FIG. 4 shows the results of an antimicrobial compound susceptibility test using BacUni QuickFISH™ staining to differentiate between intact and lysed cells. The bottom panel was treated with meropenem and the top panel is a negative control not treated with meropenem. The strain tested was K. oxy strain 43086. That strain is susceptible to meropenem and that is reflected in the significant reduction in the number of stained cells in the bottom panel compared to the top panel.

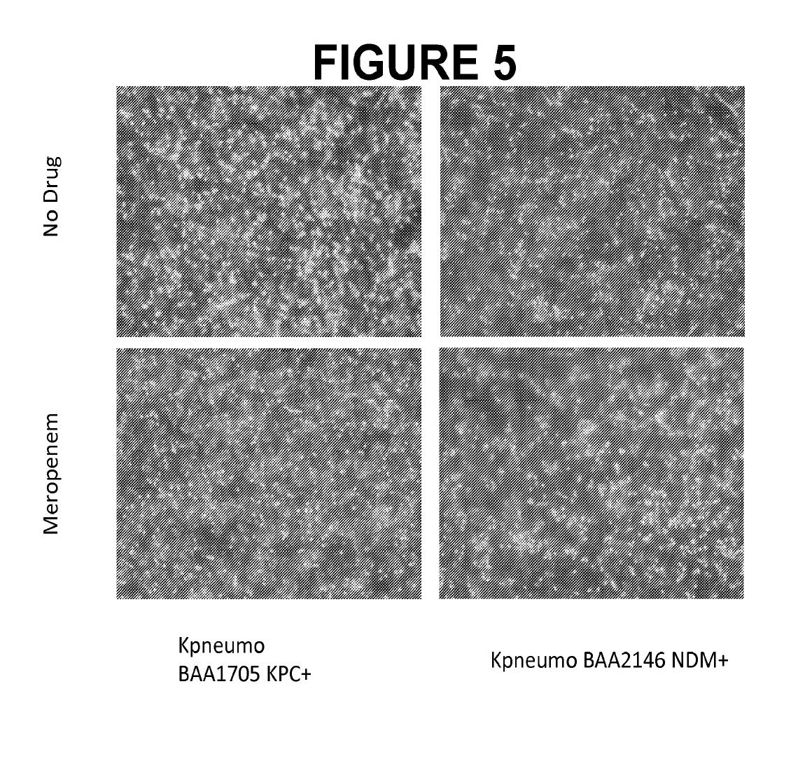

FIG. 5 shows the results of an antimicrobial compound susceptibility test using BacUni QuickFISH™ staining to differentiate between intact and lysed cells. The bottom panels were treated with meropenem and the top panels are negative controls not treated with meropenem. The left panels arc K. pneumoniae strain BAA1705 KPC+ and the right panels are K. pneumoniae strain BAA2146 NDM+. Both strains are resistant to meropenem and that is reflected in the similarity in the number of stained cells in the bottom panels (treated with meropenem) compared to the top panels (not treated with meropenem).

### Example 4

### Selective Lysis of Bacteria Exposed to Carbapenem Antimicrobial Compounds

An isolated colony from an overnight Tryptocase Soy Agar (TSA) plate for each bacteria was suspended in 1 ml Tryptocase Soy Broth (TSB) in a 5 ml BD polystyrene round bottom tube. TSB cultures were incubated at 37° C. for 1.5 hours shaking. 2500 aliquots of each stationary-phase culture were transferred to 2 ml Eppendorf microcentrifuge tubes containing 500 μl of Normal Saline (BD); or 500 μl Normal Saline with imipenem, ertapenem, or meropenem at a concentration of 10 μg/ml (6.67 μg/ml final concentration), 13.33 μg/ml (10 μg/ml final concentration), or 40 μg/ml (26.67 μg/ml final concentration). Tubes were inverted then incubated at 37° C. stagnant for thirty minutes. Then 10 μl from each tube was pipetted onto plain glass slide on a 55° C. heat block, followed by 25 μl fixation buffer (0.5% T×100, 100 mM Tris pH 9, 24% Ethanol, and 10 mM NaCl); slide is incubated until sample is dry. Ten μl of BacUni QuickFISH hybridization buffer was added to each slide followed by a 22×22 mm coverslip. The slide was then incubated at 55° C. for 15 minutes. The slides were viewed on a fluorescence microscope 60× oil immersion on Dual TxR/FITC. Images were taken with a 1 second exposure.

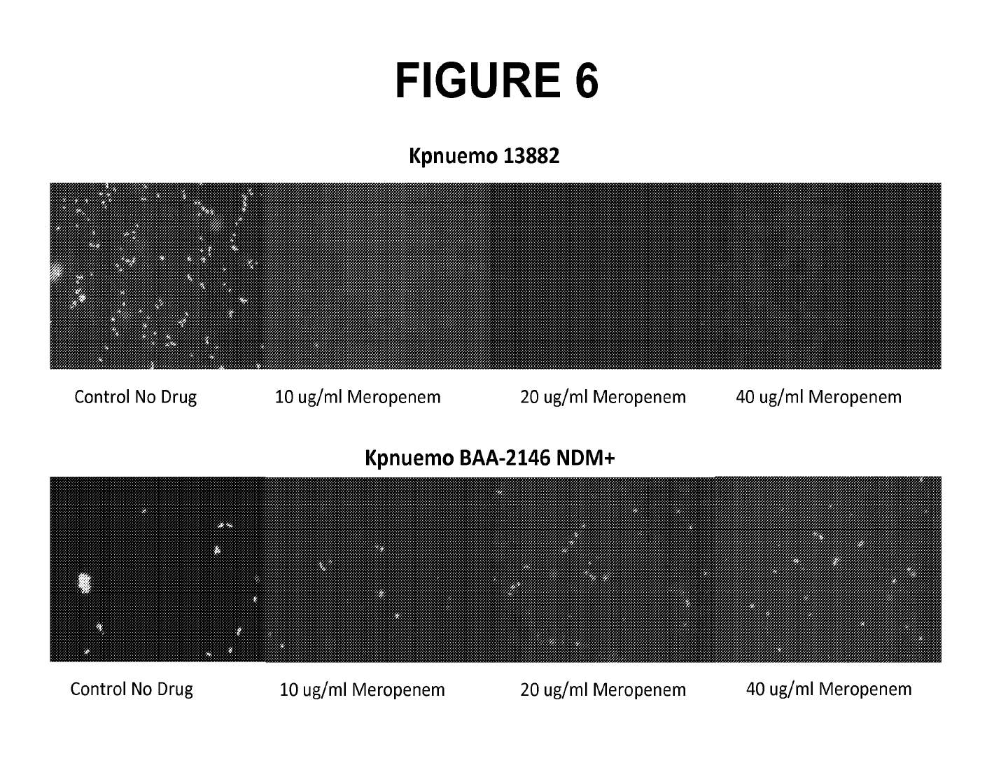

The control fixation in this experiment has all the same components, except no Triton X-100. This was done to assess whether the selective lysis step is required to resolve a difference between susceptible and resistant bacteria. The results are presented in FIGS. 6-11. The images show that without the detergent in the fixation, the susceptible bacteria remains intact and indistinguishable from the resistant strain despite exposure to antimicrobial compound.

FIG. 6 shows the results of an antimicrobial compound susceptibility test using BacUni QuickFISH™ staining to differentiate between intact and lysed cells. The top panels are the meropenem sensitive K. pneumoniae strain 13882 and the bottom panels meropenem resistant K. pneumoniae strain BAA-2146 NDM+. As indicated in the figure, negative controls not treated with meropenem are compared to samples treated with 10 μg/ml, 20 μg/ml, or 40 μg/ml of meropenem.

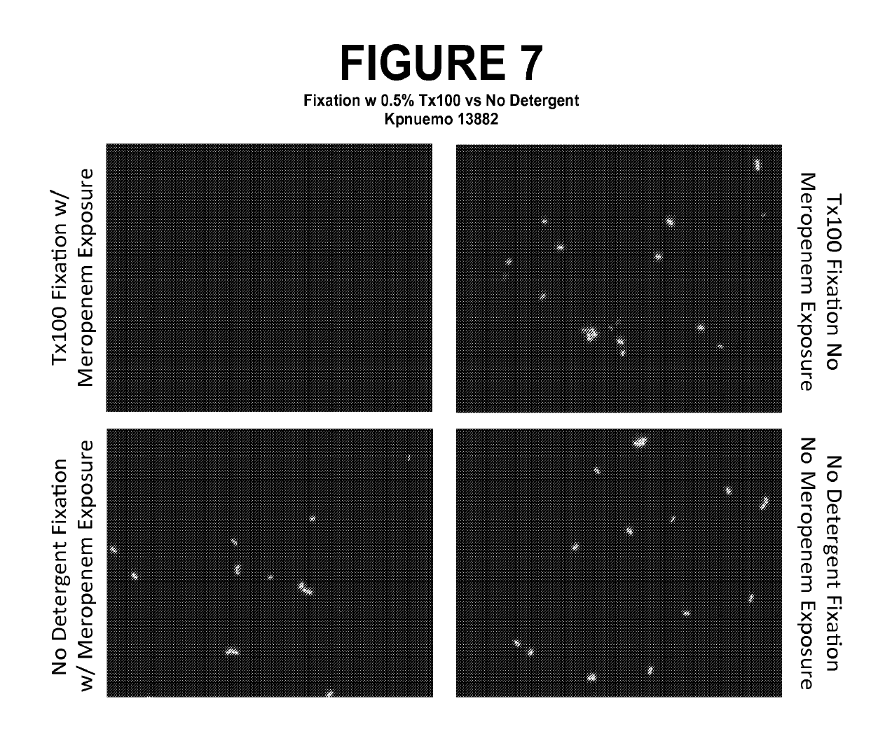

FIG. 7 shows the results of an antimicrobial compound susceptibility test using BacUni QuickFISH™ staining to differentiate between intact and lysed cells. The tested strain was meropenem susceptible K. pneumoniae strain 13882. The left panels were treated with 10 μg/ml meropenem while the right panels were not. The top panels were treated with cell wall disruption conditions comprising incubation in fixation buffer of 0.5% Triton×100, 100 mM Tris pH 9, 24% Ethanol, and 10 mM NaCl, while the bottom panels were not treated with fixation buffer. The results show that treatment with meropenem followed by exposure to fixation buffer results in the near complete absence of BacUni QuickFISH™ stained intact cells, indicating that cell lysis was extensive. In contrast, if either or both of meropenem treatment and fixation buffer exposure is omitted then stained cells are clearly present.

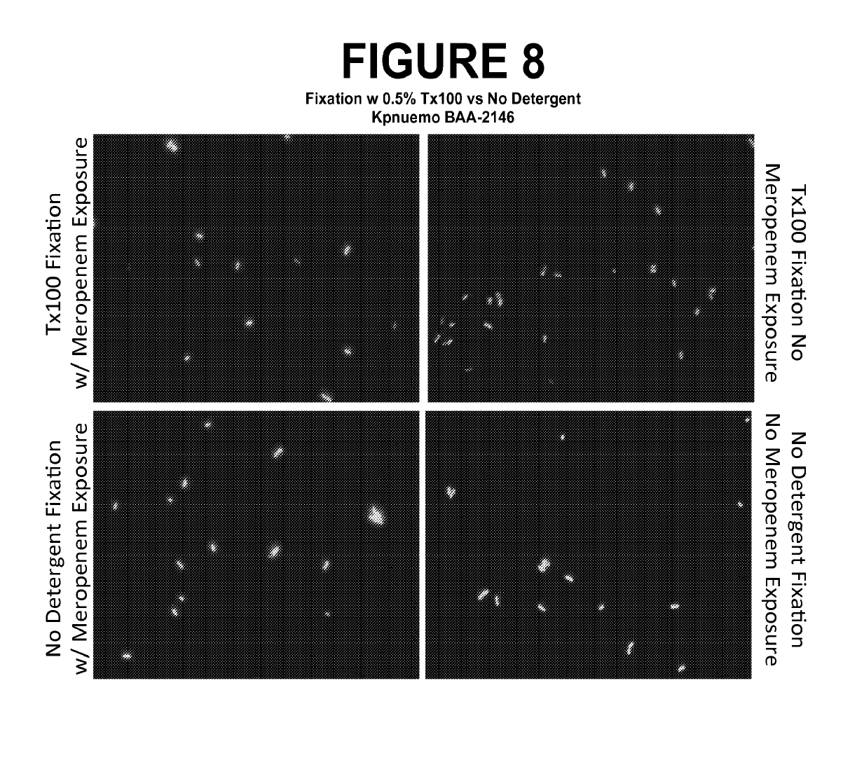

FIG. 8 shows the results of an antimicrobial compound susceptibility test using BacUni QuickFISH™ staining to differentiate between intact and lysed cells. The tested strain was meropenem resistant K. pneumoniae strain BAA2146 NDM+. The left panels were treated with 10 μg/ml meropenem, while the right panels were not. The top panels were treated with cell wall disruption conditions comprising incubation in fixation buffer of 0.5% Triton×100, 100 mM Tris pH 9, 24% Ethanol, and 10 mM NaCl, while the bottom panels were not treated with fixation buffer. The results show that treatment with meropenem followed by exposure to fixation buffer results in the near complete absence of BacUni QuickFISH™ stained intact cells, indicating that cell lysis was extensive. In contrast, if either or both of meropenem treatment and fixation buffer exposure is omitted then stained cells are clearly present.

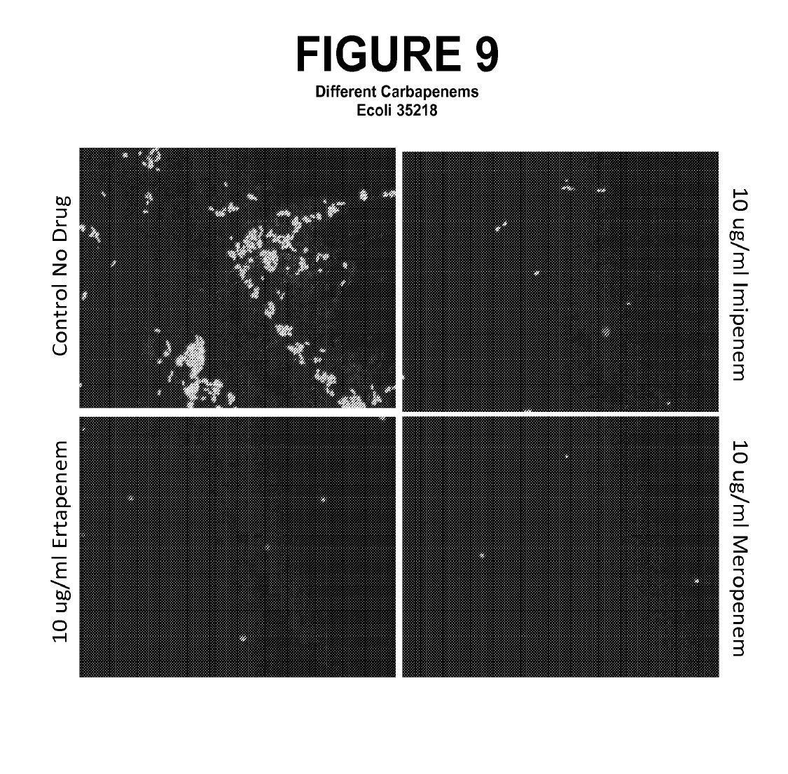

FIG. 9 shows the results of an antimicrobial compound susceptibility test using BacUni QuickFISH™ staining to differentiate between intact and lysed cells. The tested strain was E. coli strain 35218. That strain is known to be susceptible to imipenem, ertapenem, and meropenem. The upper left panel is a control not treated with any antimicrobial compound. The other panels were treated with 10 μg/ml of imipenem, ertapenem, or meropenem, as indicated. The results show that the test is able to detect susceptibility of this strain to each antimicrobial compound.

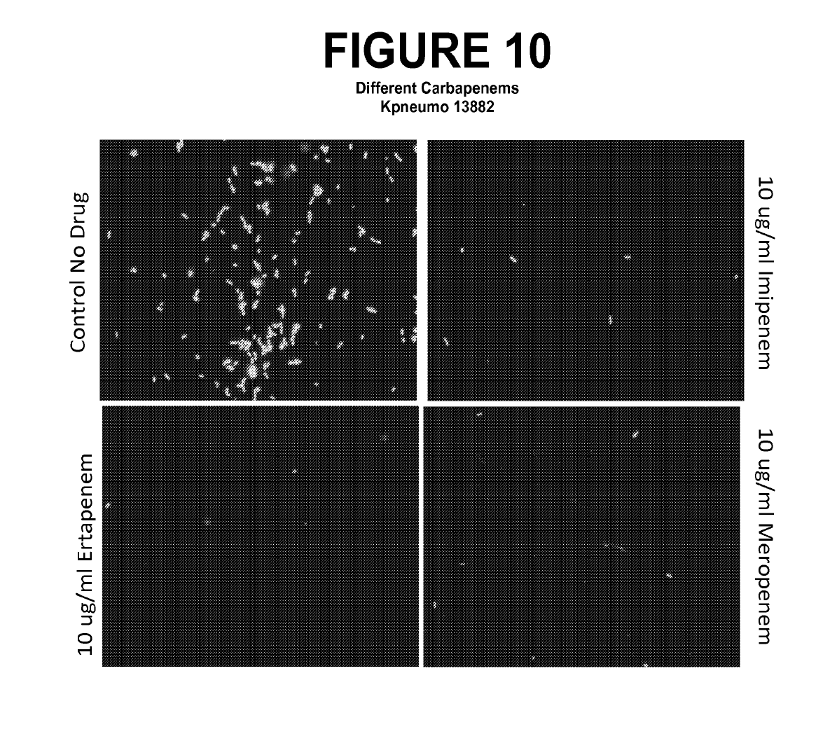

FIG. 10 shows the results of an antimicrobial compound susceptibility test using BacUni QuickFISH™ staining to differentiate between intact and lysed cells. The tested strain was K. pneumoniae strain 13882. That strain is known to be susceptible to imipenem, ertapenem, and meropenem. The upper left panel is a control not treated with any antimicrobial compound. The other panels were treated with 10 μg/ml of imipenem, ertapenem, or meropenem, as indicated. The results show that the test is able to detect susceptibility of this strain to each antimicrobial compound.

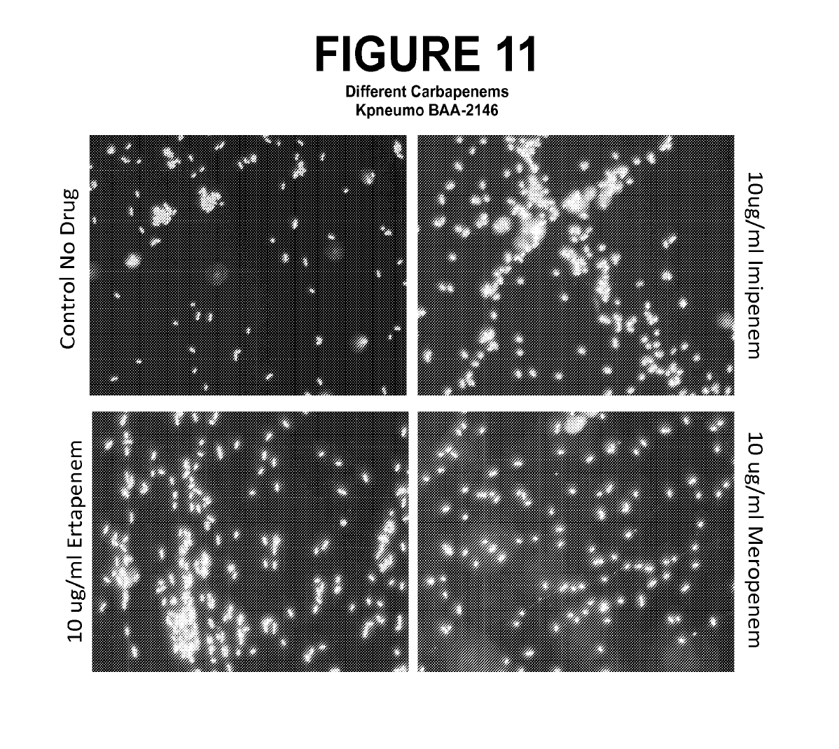

FIG. 11 shows the results of an antimicrobial compound susceptibility test using BacUni QuickFISH™ staining to differentiate between intact and lysed cells. The tested strain was K. pneumoniae strain BAA2146 NDM+. That strain is known to be resistant to imipenem, ertapenem, and meropenem. The upper left panel is a control not treated with any antimicrobial compound. The other panels were treated with 10 μg/ml of imipenem, ertapenem, or meropenem, as indicated. The results show that the test is able to detect resistance of this strain to each antimicrobial compound.

### Example 5

### Flow Cytometry

An isolated colony from overnight Tryptocase Soy Agar (TSA) plate for each bacteria was suspended in 1 ml Tryptocase Soy Broth (TSB) in a 5 ml BD polystyrene round bottom tube. TSB cultures were incubated at 37° C. overnight. Ten μl of overnight culture was inoculated into 1 ml TSB and incubated shaking at 37° C. for three hours. Two 250 μl aliquots of each log-phase culture were transferred to two 2 ml Eppendorf microcentrifuge tubes. One tube contained 500 μl of Normal Saline (BD); the second tube contained 504.1 Normal Saline with meropenem at 10 μg/ml. Tubes were inverted then incubated at 37° C. stagnant for thirty minutes. An aliquot was removed at this point for flow cytometry measurement of the total cell count. Then 250 μl lysis buffer (0.5% SDS in PSB) was added to each tube and each tube was vortexed for 5 seconds. Post incubation, all tubes were spun at 10000 G for 5 minutes. Supernatant was decanted and the pellet resuspended in Normal Saline. Tubes were vortexed.

Re-suspended pellets were then diluted 20 times into a solution of 10 mM Tris-HCl pH=7.6, containing 2.5 μM SYTO 9 (stain permeable to all bacterial cells). Stained bacterial cells were analyzed by flow cytometry using the Guava EasyCyte™ Mini System equipped with a 488 nm diode laser, capillary flow cell, forward and side scatter detectors and fluorescence (green, yellow and red) detectors. Flow cytometry dot plots were acquired with CytoSoft (Guava ExpressPlus) software and cell counts were obtained. The results are shown in Table 4.

TABLE 4

| | Antimicrobial | Cell | | R |
|---|---|---|---|---|
| Strain | compound | Number/ml | Stage | or S |
| K. pneumo | — | 3.04 × 10⁶ | Pre-lysis | — |
| BAA2146 (NDM+) | | | | |
| K. pneumo | 10 μg/ml | 3.04 × 10⁶ | Pre-lysis | — |
| BAA2146 (NDM+) | Meropenem | | | |
| K. pneumo | — | 2.67 × 10⁶ | Post-lysis + | R |
| BAA2146 (NDM+) | | | spin | |
| K. pneumo | 10 μg/ml | 2.83 × 10⁶ | Post-lysis + | R |
| BAA2146 (NDM+) | Meropenem | | spin | |
| K. pneumo 13882 | — | 3.45 × 10⁶ | Pre-lysis | — |
| (NDM−) | | | | |
| K. pneumo 13882 | 10 μg/ml | 2.38 × 10⁶ | Pre-lysis | — |
| (NDM−) | Meropenem | | | |
| K. pneumo 13882 | — | 3.23 × 10⁶ | Post-lysis + | S |
| (NDM−) | | | spin | |
| K. pneumo 13882 | 10 μg/ml | 2.41 × 10⁵ | Post-lysis + | S |
| (NDM−) | Meropenem | | spin | |

The first four rows present data for the meropenem resistant strain K. pneumoniae BAA2146 (NDM+). The sample treated with meropenem and the control sample each had 3.04×10⁶ cells at the pre-lysis stage. After lysis and centrifugation the control sample contained 2.67×10⁶ cells (12% reduction) while the meropenem treated sample contained 2.83×10⁶ cells (7% reduction). Those differences are deemed insignificantly different from each other. This result is consistent with the known resistance of strain K. pneumoniae BAA2146 (NDM+) to meropenem.

Rows five through eight present data for the meropenem sensitive strain K. pneumoniae 13882 (NDM−). The sample treated with meropenem and the control sample had 2.38×10⁶ cells and 3.45×10⁶ cells at the pre-lysis stage, respectively. After lysis and centrifugation the control sample contained 3.23×10⁶ cells (6% reduction) while the meropenem treated sample contained 2.41×10⁵ cells (90% reduction). Those differences are deemed significantly different from each other, and demonstrate that this strain is susceptible to meropenem. This result is consistent with the known resistance of strain K. pneumoniae BAA2146 (NDM+) to meropenem.

In the remaining examples, isolates harboring know resistance genes were tested by the method of the invention. As a way of organizing the data, the test results are presented by enzymatic class. When put into practice on fresh clinical isolates, there will be no a priori knowledge of the class or classes of enzymes, or non-enzymatic mechanisms of resistance harbored with a particular strain.

### Example 6

### Class A Beta-Lactamases

The strains used in this example and the following examples were obtained from a repository. Beta-lactamase characterization was performed at their site using either assays from Check-Points (Check-Points, the Netherlands) or in-house multiplex PCR.

In this example, isolates tested include strains with and without carbapenemases. From fresh blood agar plates, three colonies per strain were used to inoculate blood culture media containing human blood and grown overnight at 37° C. with shaking, producing a simulated blood culture. Cultures were then diluted 1:10 into broth medium and grown for an additional hour at 37° C. with shaking. For each strain, four 250 μL aliquots were added into four separate 2 mL Eppendorf microcentrifuge tubes. The first tube contained 500 μl of DEPC-treated water; the second tube contained 10 μg/mL meropenem in 500 μL of DEPC-treated water; the third tube contained 10 μg/mL ertapenem in 500 μL of DEPC-treated water; the fourth tube contained 10 μg/mL imipenem in 500 μL of DEPC-treated water—all tubes yielding a final concentration of 6.67 μg/mL of antibiotic with the addition of the culture. Tubes were inverted to mix and subsequently incubated without shaking at 35° C. for 60 minutes. Following incubation, 250 μl, of lysis buffer (0.5% SDS in 1×PBS) was added to each tube and mixed by inversion. Samples were incubated at room temperature for five minutes, and then centrifuged at 10,000×g for five minutes. The supernatant was decanted and the pellet resuspended in 500 μL of normal saline. Tubes were vortexed to resuspend the pellet completely, and then OD₆₀₀ measurement was taken and the percent change, or percent lysis, between the control and antibiotic tubes was calculated (percent change=[control OD−antibiotic OD]/control OD). Results were then compared to the susceptibility of the strains, determined using the Clinical and Laboratory Standards Institute (CLSI) methods for disk diffusion. For this set of examples, ≦30% change was considered resistant, while ≧70% change was considered sensitive.

Of note, if a strain was determined to be intermediate to a drug by the disk diffusion method using CLSI breakpoints, it was expected to correlate to the resistant answer by the method of the invention. This was decided based on the definition of intermediate, in short that the antibiotic tested may work on the strain if high enough levels of antibiotic are achieved at the site of infection; however, there is a chance the treatment may fail in this situation. With this consideration, it is preferable that intermediate strains are conservatively scored as resistant to prevent possible treatment failure. The ability to easily design the assay in this way is one useful feature of certain embodiments of the invention.

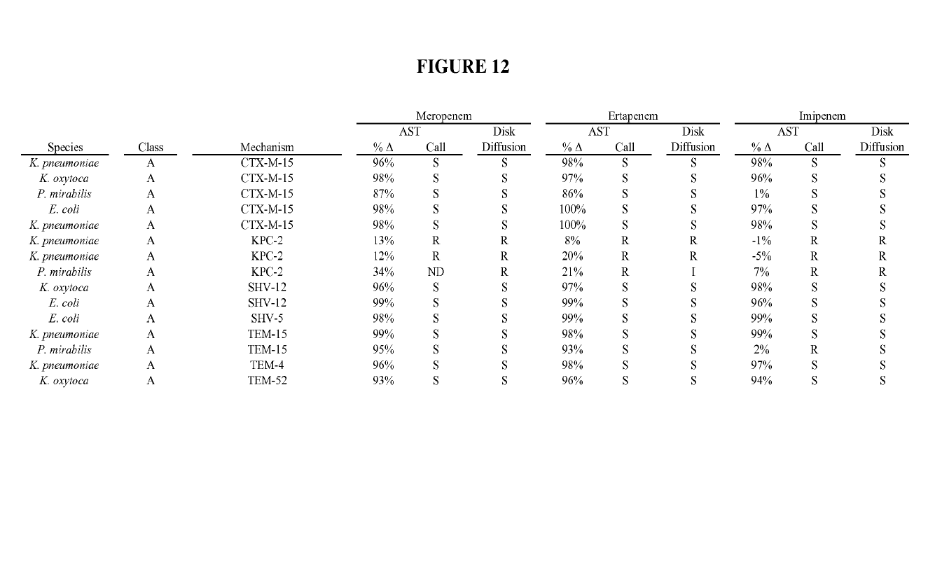

For the class A beta-lactamase strains tested, there was a 93% concordance with meropenem and imipenem and 100% concordance with ertapenem between the tested method and the disk diffusion results (FIG. 12). Firstly, this example demonstrates that the tested method can be run on simulated blood cultures, a primary human sample; whereas routine susceptibility testing methods require an isolated sample and overnight incubation. For meropenem, the strain that did not give the appropriate result was a P. mirabilis with a KPC gene. This strain should be resistant according to the disk diffusion result and was called sensitive by the tested method. However, there was only slightly too much lysis to call the strain resistant and not enough to call it sensitive by the tested method, hence the ND (not determined) designation reported in FIG. 12. For imipenem, the strain that did not yield the correct result was also a P. mirabilis strain, which gave a false resistant susceptibility. While false results are never desirable, a false resistance answer still provides the patient with antimicrobial therapy that will likely be successful, though it may be overly aggressive. Over-treatment is to be avoided when possible as it may lead to increased resistance in general, but is preferable to under-treatment for the patient at hand. The reason for the false result is unknown; there may be species-specific phenotypic attributes which prevent efficient lysis that have yet to be identified. The results reported in this example demonstrate that the method of the invention is useful for antibiotic susceptibility testing of class A beta-lactamase strains. These data strongly suggest that use of the test method directly from blood culture would be advantageous to the patient as it would provide useful, accurate guidance for presumptive therapy in a significantly reduced timeframe as compared to conventional methods. Additionally, for the most clinically relevant Gram-negative species (E. coli, K. pneumoniae) and the most commonly encountered class of beta lactamases, Class A, the accuracy of the method is very high. As with other susceptibility tests, knowledge of the species (identification) greatly increases the value and accuracy of the result.

### Example 7

### Class B Beta-Lactamases

This example follows the design of Example 6; however, for the one hour culture step, zinc sulfate was added to the culture media to aide in the expression of the metallo-beta-lactamases.

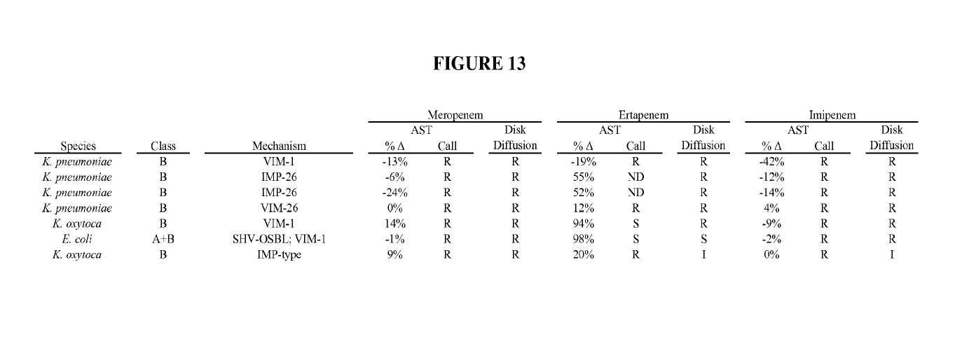

As seen in Example 6, this method for determining organism susceptibility was successfully performed on samples in human blood culture, not isolated samples. The data are presented in FIG. 13 and show that for the Class B beta-lactamases there was a 100% concordance with meropenem and imipenem and a 57% concordance with ertapenem. In this example, a strain was used which contained two beta-lactamases, though only the VIM-1 (the Class B enzyme) confers resistance to carbapenems. This strain performed as expected with all three carbapenems, demonstrating that the presence of additional resistance mechanisms in a strain does not interfere with the assay. It is unknown why not all of the strains functioned as expected with ertapenem. The susceptibility of two of the strains was not determined (ND) and the third was falsely called sensitive. It is possible, that even with the addition of the zinc, 1 hour is not enough time for the metallo-beta-lactamases to be expressed at a level which would hydrolyze the antibiotics, preventing unexpected lysis. As the Class B beta-lactamases are zinc-dependent, the supplementation of zinc increases the activity of the Class B beta-lactamases, allowing for rapid antibiotic hydrolysis. Increasing the supplemented zinc to a higher concentration could aide with the detection of correct susceptibility. The results reported in this example demonstrate that the tested method of the invention is useful for antibiotic susceptibility testing of class B beta-lactamase strains.

### Example 8

### Class C Beta-Lactamases

This example follows the design of Example 6.

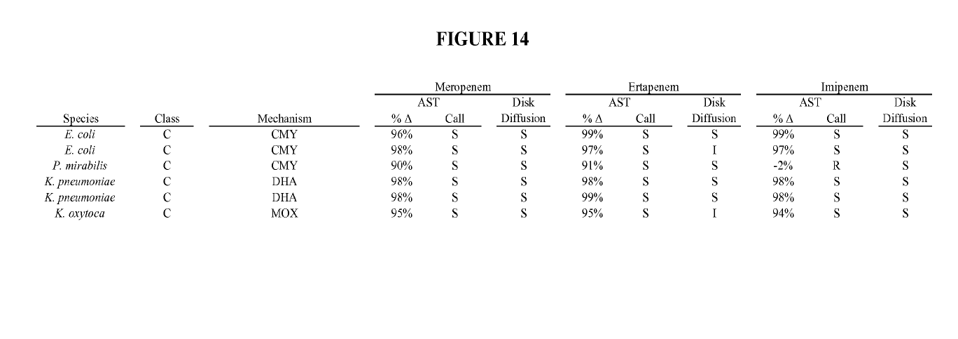

As shown in FIG. 14, for the class C beta-lactamases, there was a 100% concordance with meropenem, a 67% concordance with ertapenem, and an 83% concordance with imipenem. With the two strains that were false sensitive, it is possible that in a short antibiotic exposure time they would not be detected. It is possible these AmpCs are inducible and the one hour exposure is not long enough to turn on the mechanisms to produce enough enzyme to destroy the antibiotic, thereby preventing lysis. The strain that gave a false resistance result was another P. mirabilis strain. As stated in Example 6, it is possible there is a species specific mechanism that is preventing the uptake of antibiotic or the lysis of the cells. An induction step, using low levels of antibiotic known to induce the expression of AmpCs (such as cefoxitin), prior to the antibiotic exposure would increase the likelihood of Class C beta-lactamase production and successful susceptibility identification. The results reported in this example demonstrate that the tested method of the invention is useful for antibiotic susceptibility testing of Class C beta-lactamase strains.

### Example 9

### Class D Beta-Lactamases

The methods of this example were the same as those used in Example 6.

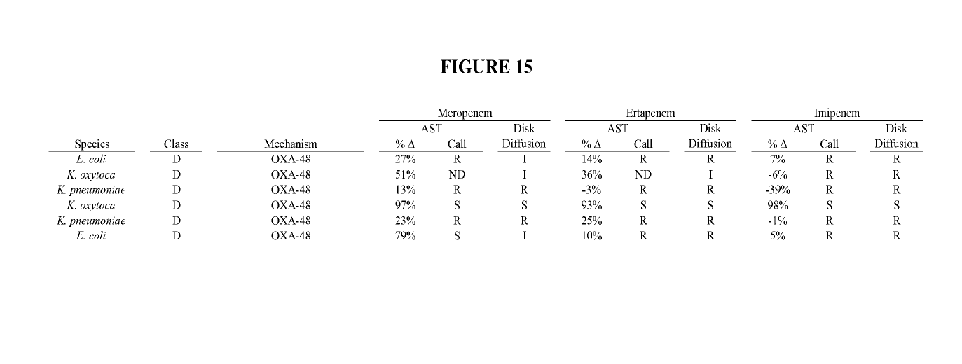

As shown by the data reported in FIG. 15, when compared to the disk diffusion results, the assay of Class D beta-lactamases gave 67% concordance with meropenem, 83% concordance with ertapenem, and 100% concordance with imipenem. Because there is 100% concordance with imipenem, this example demonstrates that the method of the invention works on Class D beta-lactamases. One strain was incorrect with ertapenem; however, there was only slightly too much lysis (36%) to call the strain resistant. The two that were incorrect with meropenem were both false sensitives, having too much lysis to be called sensitive. The OXA enzymes have weak carbapenem-hydrolyzing activity and therefore other factors of the method may need to be adjusted, such as antibiotic exposure (time and/or concentration), to optimize the assay for all carbapenems.

### Example 10

### Other Mechanisms of Resistance

The methods of this example were the same as in Example 6; however, the antibiotic exposure time was two hours instead of 60 minutes.

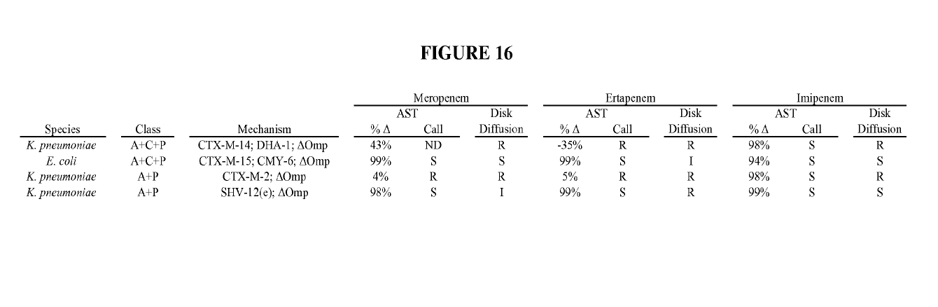

As shown by the data reported in FIG. 16, all three carbapenems tested had a 50% concordance compared to the disk diffusion data. For meropenem, there was a strain that was called ND, with too much lysis to be resistant, but not close to being sensitive. This strain, which also gave a false sensitive result for imipenem, also contains a Class C beta-lactamase. In order for the resistance to express completely, this strain may need to be induced. The rest of the incorrect results were all false sensitive. Increasing the antibiotic exposure time to more than two hours may increase the number of accurate results, as increasing the time from 60 minutes improved the assay.

### Example 11

### Detection in Spiked Urine and Bronchoalveolar Lavage Samples

In order to demonstrate that the methods of the invention work in patient sample types other than blood culture, urine and bronchoalveolar lavage (BAL) samples were obtained from a microbiology lab and spiked with lab strains to simulate true infections. These samples were obtained from actual patients and yielded no growth by routine methods. In order to add organisms, seven strains used in prior examples were tested. Two negative urines and two negative BALs were tested.

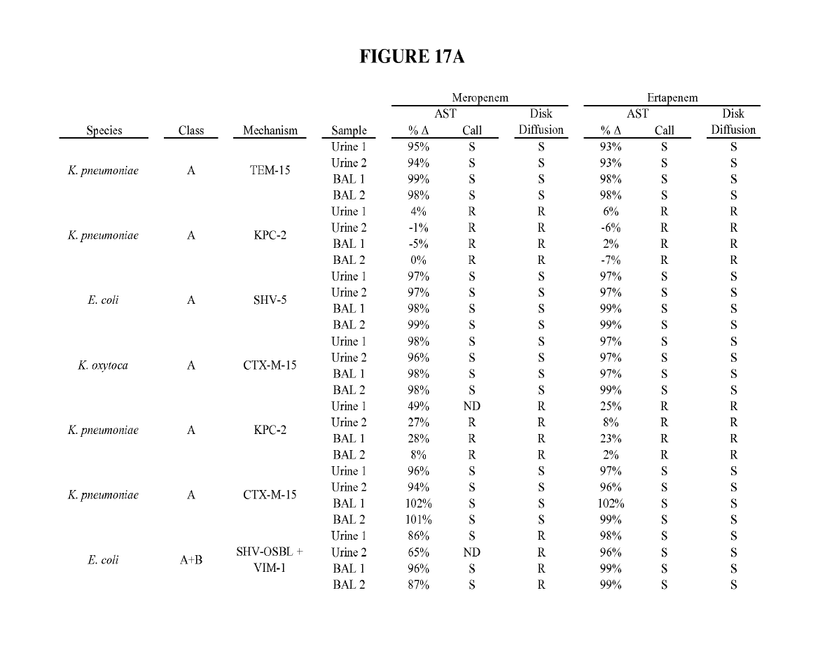

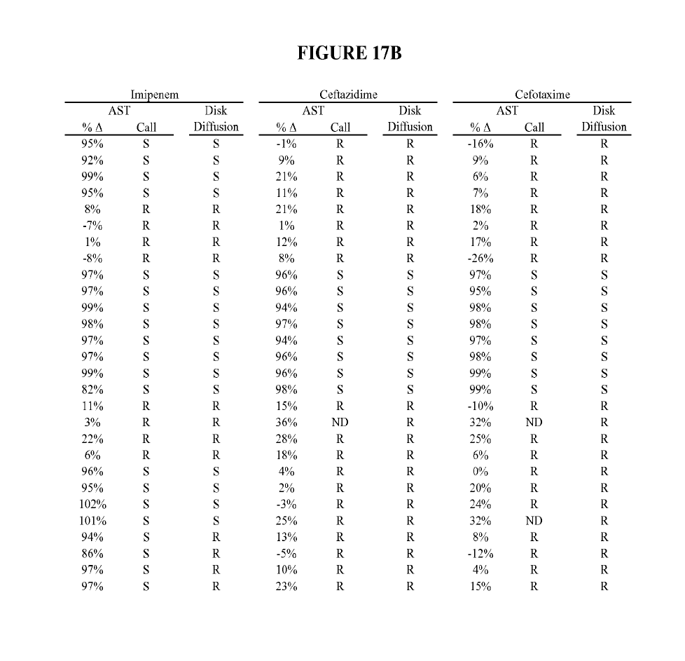

The strains were inoculated into enriched broth media by selecting several colonies from an overnight blood agar plate. The inoculated cultures were then grown overnight at 37° C. with shaking Each culture was split into four aliquots and spun down 10,000×g for five minutes and decanted. Each aliquot was resuspended with either one of the urines or one of the BALs in equal volume of what was spun down. The rest of the protocol was the same as in Example 6, except with the addition of two cephalosporin antibiotics. In addition to the four tubes prepared (one control and three with carbapenems), the fifth tube contained 750 μg/mL imipenem in 500 μL of DEPC-treated water and the sixth tube contained 750 μg/mL imipenem in 500 μL of DEPC-treated water—both yielding a final concentration of 500 μg/mL with the addition of the culture. Results were compared to the disk diffusion method. The data are reported in FIGS. 17A and 17B.

Both simulated urines and BALs worked successfully in this method. For the carbapenems, meropenem had an 82% concordance, ertapenem had a 100% concordance, and imipenem had an 86% concordance with the disc diffusion method. The strain that gave false sensitive results for meropenem and imipenem was a Class B beta-lactamase. This strain performed as expected in prior experiments, but may have lost the plasmid which coded for its Class B enzyme while under non-selective culture in the days prior to this experiment. Strains with Class B plasmids are known for losing plasmids in the absence of selective pressure in laboratory environments. The other strain that did not perform as expected in meropenem was called ND for only one of the four sample types. This strain gave the expected susceptibility for the rest of the sample types for meropenem and for all four sample types with the other four drugs tested.

For the cephalosporins, ceftazidime had a 96% concordance and cefotaxime had a 93% concordance. The three data points that did not agree with disc diffusion were all scored ND, and had slightly too much lysis (36%, 32%, and 32%) to be called resistant, however, were not close to being called sensitive. It is probable that minor method modifications will be sufficient to optimize the method performed from BAL or urine samples such that the strains perform as expected.

### Example 12

### Testing of Extended-Spectrum Beta-Lactamases Against Clinical Enterobacteriaceae Strains With Characterized Mechanisms of Resistance

Several clinically characterized Enterobacteriaceae strains were obtained from a repository. These strains harbored plasmids encoding several carbapenemases and extended spectrum beta-lactamases. Some of the strains had porin deletions. Three colonies were picked from blood agar plates and used to inoculate broth supplemented with blood and incubated overnight at 37° C. with shaking A ten-fold dilution was made into broth media and incubated for one hour with shaking After one hour, 250 μl were transferred into three different Eppendorf microcentrifuge tubes. A control tube containing 500 μl of DEPC-treated water, a second tube 500 μl of DEPC-treated water with 750 μg/ml of cefotaxime (final concentration of 500 μg/ml) and the third tube 500 μl of DEPC-treated water with 750 μg/ml of ceftazidime (final concentration of 500 μg/ml). The tubes were inverted to ensure a good mixing and incubated for one hour at 35° C. without shaking. In some of the strains with class D enzymes, exposing the bacteria to the antibiotic for two hours improved the agreement between the results obtained with methods of the invention and those obtained by disk diffusion. After the incubation period was over, 250 μL of lysis buffer (0.5% SDS in 1×PBS) was added to each tube and mixed by inversion. The tubes were incubated for five minutes at room temperature and then centrifuged for five minutes at 10,000×g to pellet the cells. The supernatant was removed and the pellet resuspended in 500 μL of normal saline by vortex. The OD₆₀₀ was measured and the percentage of lysis calculated and cut-offs were set at ≦30% lysis to identify resistance and ≧70% for sensitive. If a strain was determined to be intermediate to a drug by the disk diffusion method, it was expected to correlate to the resistant answer by the method of the invention. This was decided based on the definition of the intermediate, in short that the antibiotic tested may work on the strain if high enough levels of antibiotic are achieved at the site of infection; however, there is a chance the treatment may fail. With this consideration, it is preferable that intermediate strains are conservatively scored as resistant to prevent possible treatment failure.

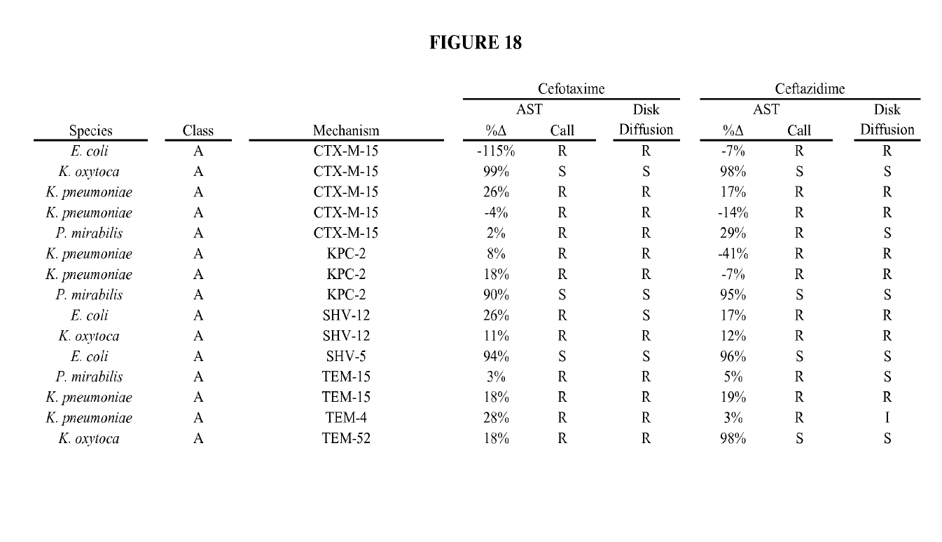

The results were then compared to the disk diffusion results, obtained using CLSI methods. Agreement of 93% and 87% for Class A beta-lactamases were found for cefotaxime and ceftazidime, respectively. The experimental results are reported in FIG. 18. All three strains for which the method did not agree with disk diffusion assay were classified as “false-resistant”. This variation may be due to individual characteristics of each isolate such as the presence of efflux pumps, mutations in the target proteins, or use of alternate penicillin binding proteins. The data obtained shows that this methodology can be efficiently used in the detection of susceptibility in strains harboring Class A enzymes and efficiently contribute to a positive treatment.

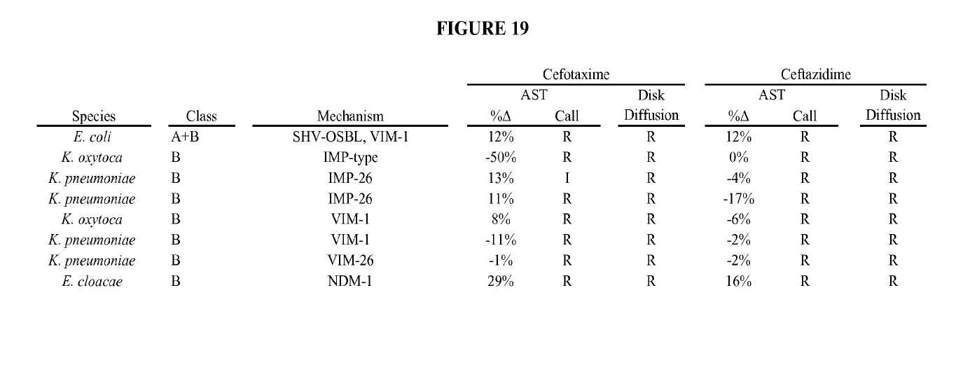

The results obtained with strains known to express Class B beta-lactamases are reported in FIG. 19 and show a 100% agreement with the disk diffusion assay and demonstrate the effectiveness of this methodology in predicting the susceptibility profile of strains carrying Class B beta-lactamases.

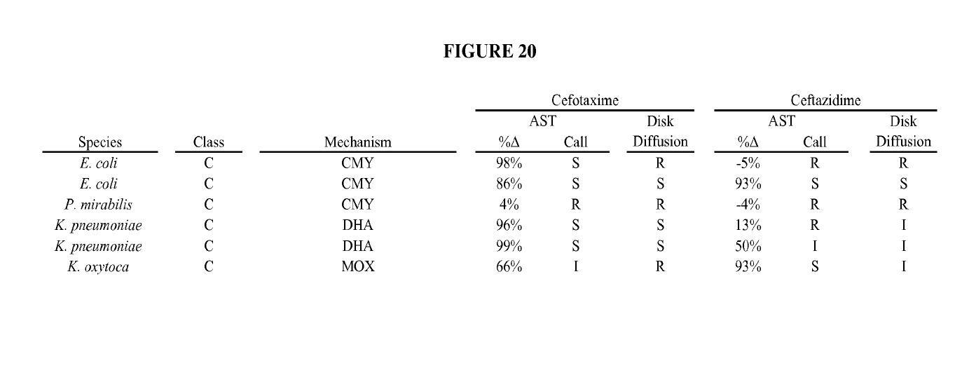

The results of our assay when testing strains harboring plasmid encoded Class C beta-lactamases are presented in FIG. 20 and show a 67% agreement with the results obtained for cefotaxime and 83% for ceftazidime. An E. coli strain encoding a CMY enzyme and a K. oxytoca encoding a MOX gene were the only strains tested that were classified as sensitive/intermediate respectively while they were both classified as resistant by the disk diffusion assay.

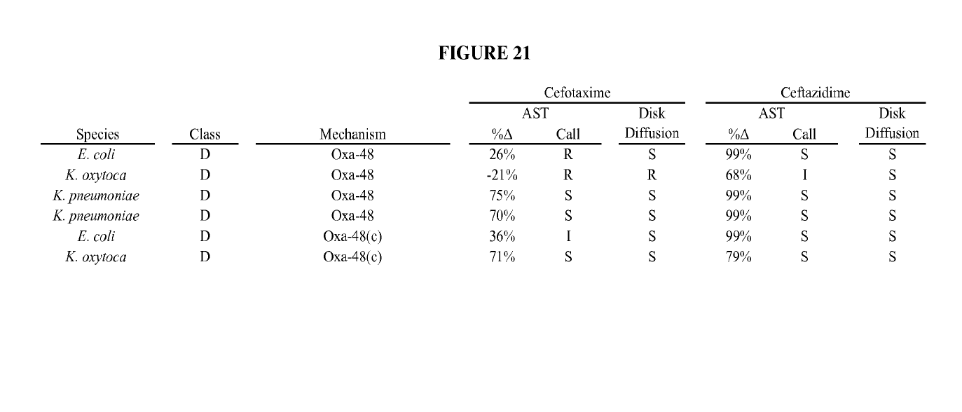

The data presented in FIG. 21 show that there is an agreement of 67% for cefotaxime and 83% for ceftazidime between this assay and the disk diffusion test for strains with Class D enzymes. Neither of the E. coli isolates agreed with the data obtained for cefotaxime. Experiments made with ceftriaxone gave similar results to those obtained with cefotaxime. The CLSI considers cefotaxime and ceftriaxone interchangeable, since they share very similar properties. This agreement in the results suggests that some event may be happening at a cellular level that hinders the cellular lysis by cefotaxime/ceftriaxone in this species. The percentage of lysis of the strain that did not give a consistent result for ceftazidime and was very close to the defined cutoff. Improvements in the methodology will likely identify this susceptibility, and in a clinical environment would indicate to the clinician that ceftazidime could be used, even though the strain is resistant to cefotaxime. This data shows that this method works well when testing strains with Class D enzymes.

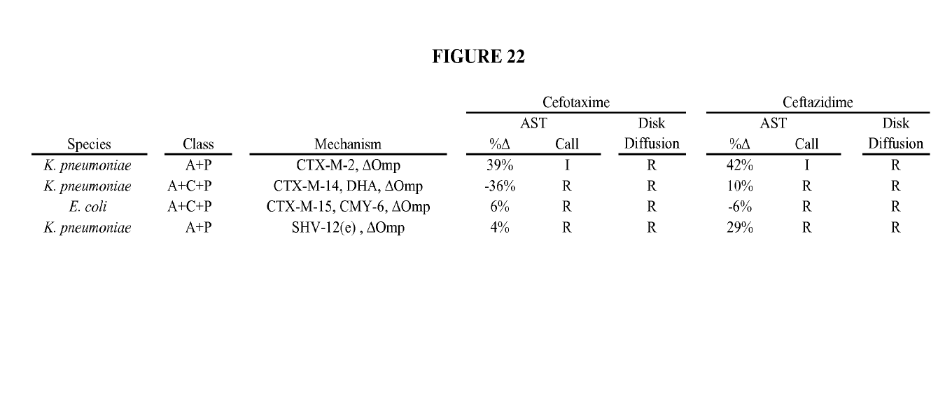

FIG. 22 displays the results of testing strains with multiple known mechanisms for resistance to beta lactamases. The agreement between this assay and the disk diffusion test for the strains harboring beta-lactamases together with other mechanisms of resistance was of 75% for the data obtained for cefotaxime and ceftazidime. The phenotype of the strains that were not properly identified was classified as intermediate. In the cases where intermediate strains are identified, clinicians usually decide on other treatments, as there is some resistance present and treatment may fail. Therefore the result given by the method of the invention would not jeopardize the clinical outcome. These data clearly indicate that this assay is also able to identify these strains that not only harbor plasmid encoded beta-lactamases, but also those strains that express simultaneously non-specific mechanisms of resistance.

The results obtained with this experiment clearly indicate that this method is effective when testing the susceptibility of diverse species of bacteria to the clinically relevant extended-spectrum beta-lactams ceftazidime and cefotaxime. It provides satisfactory results with a diverse species of bacteria encoding genes to the four classes of beta-lactamases. It also demonstrates that it works when non-specific resistance mechanisms are present in simultaneous with beta-lactamases and when there are two different beta-lactamases present. In a clinical setting, these results would likely contribute to a successful clinical outcome by providing rapid and valuable information regarding the susceptibility profile of diverse strains to extended-spectrum beta-lactamases.

The data presented in Examples 6-12 demonstrate the success of this method. Some of the classes performed at 100% concordance with the gold standard, while others were not at the same level. The method of the invention determines susceptibility, rather than simply identifying the presence of a gene as do PCR-based technologies; therefore, both sensitive and resistant strains can be identified due to the activity (or lack thereof) of their resistance mechanisms. Examples with lower agreements, such as the porin mutations with the carbapenem antibiotics, still demonstrate the functionality of the assay, for both sensitive and resistant strains. With further optimization of this methodology, high level of concordance is expected with all resistance mechanisms and all antibiotics tested.

While various embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention.

1. A method of determining whether a target bacteria is susceptible to an at least one antimicrobial compound, comprising:
providing a sample comprising the target bacteria;
maintaining the sample in the presence of at least one antimicrobial compound to provide an antimicrobial compound-exposed target bacterial sample;
exposing the antimicrobial compound-exposed target bacterial sample to a cell-wall disruption condition; and
determining the level of lysis and/or the level of remaining intact cells present in the antimicrobial compound-exposed target bacterial sample;
wherein the method is performed such that the level of lysis and/or remaining intact cells is determined without determining lysis or non-lysis on a cell-by-cell basis.

2. The method of claim 1, wherein the target bacteria are not immobilized during the exposure to cell-wall disruption conditions.

3. The method of claim 1, further comprising comparing the level of lysis and/or the level of remaining intact cells present in the antimicrobial compound-exposed target bacterial sample to a reference level to score the sample as sensitive or resistant to the at least one antimicrobial compound.

4. The method of claim 1, wherein the method does not comprise detecting the presence or absence of at least one target bacteria protein and/or at least one target bacteria nucleic acid.

5. The method of claim 1, wherein the sample comprising the target bacteria is a primary sample.

6. The method of claim 1, wherein the sample comprising the target bacteria is an in vitro cultured sample.

7. The method of claim 6, wherein the in vitro cultured sample is provided by obtaining a subject sample comprising the target bacteria and culturing target bacteria in the subject sample to provide the in vitro cultured sample.

8. The method of claim 1, wherein the target bacteria is Gram-negative.

9. The method of claim 8, wherein the target bacteria is rod-shaped.

10. The method of claim 8, wherein the target bacteria is a member of the family Enterobacteriaceae.

11. The method of claim 8, wherein the target bacteria is a non-fermenter bacterium.

12. The method of claim 1, wherein the at least one antimicrobial compound is a bactericidal antimicrobial compound.

13. The method of claim 1, wherein the at least one antimicrobial compound comprises a β-lactam ring.

14. The method of claim 1, wherein the at least one antimicrobial compound is a carbapenem.

15. The method of claim 1, wherein the at least one antimicrobial compound is selected from colistin or a derivative thereof, tigecycline or a derivative thereof, a cephalosporin or a derivative thereof, a carbapenem or a derivative thereof, cefoxitin or a derivative thereof, and fosfomycin or a derivative thereof.

16. The method of claim 1, wherein the sample is maintained in the presence of a concentration of the at least one antimicrobial compound that is at least the minimum inhibitory concentration of the at least one antimicrobial compound.

17. The method of claim 1, wherein the sample is maintained in the presence of the at least one antimicrobial compound for about two hours or less.

18. The method of claim 1, wherein the cell-wall disruption condition comprises at least one of a detergent, a physical means of disrupting cells, alkaline conditions, a chemical cell-wall disruption agent, and an enzyme.

19. The method of claim 18, wherein the cell-wall disruption condition comprises a detergent and a physical means of disrupting cells.

20. The method of claim 18, wherein the detergent is selected from at least one of Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

21. The method of claim 1, wherein if the level of lysis present in the antimicrobial compound-exposed target bacterial sample is at or above a reference level and/or the level of remaining intact cells present in the antimicrobial compound-exposed target bacterial sample is at or below a reference level, the target bacteria are scored as sensitive to the antimicrobial compound.

22. The method of claim 1, wherein if the level of lysis present in the antimicrobial compound-exposed target bacterial sample is not at or above a reference level and/or the level of remaining intact cells present in the antimicrobial compound-exposed target bacterial sample is not at or below a reference level, the target bacteria are scored as resistant to the antimicrobial compound.

23. The method of claim 1, further comprising:
providing a sample comprising the target bacteria;
maintaining the sample in the absence of the at least one antimicrobial compound to provide an antimicrobial compound-negative control target bacterial sample;
exposing the antimicrobial compound-negative control target bacterial sample to the cell-wall disruption condition; and
determining the level of lysis and/or the level of remaining intact cells present in the antimicrobial compound-negative control target bacterial sample.

24. The method of claim 23, further comprising comparing the level of lysis and/or the level of remaining intact cells present in the antimicrobial compound-exposed target bacterial sample to the level of lysis and/or the level of remaining intact cells present in the antimicrobial compound-negative target bacterial sample.

25. The method of claim 1, wherein the time elapsed between the beginning of maintaining the sample in the presence of the at least one antimicrobial compound to the determination of whether the cell-wall disruption condition lyses target bacterial cells present in the antimicrobial compound-exposed target bacterial sample is three hours or less.

26. A method of treating a bacterial infection in a subject, comprising:
determining that a target bacteria is susceptible to an antimicrobial compound by the method of claim 1; and
administering a therapeutically effective amount of the at least one antimicrobial compound to the subject to thereby treat the bacterial infection in the subject.

27. A method of screening a candidate compound to identify a compound having antimicrobial activity against a target bacteria, comprising:
providing at least one sample of a target bacteria;
maintaining the at least one of sample of the target bacteria in the presence of at least one candidate compound to provide at least one candidate antimicrobial compound-exposed target bacterial sample;
exposing the at least one candidate compound-exposed target bacterial sample to a cell-wall disruption condition; and
determining whether the cell-wall disruption condition lyses target bacterial cells present in the at least one candidate antimicrobial compound-exposed target bacterial sample;
wherein the method is performed such that the level of lysis and/or remaining intact cells is determined without determining lysis or non-lysis on a cell-by-cell basis.

28. The method of claim 27, wherein the target bacteria are not immobilized during the exposure to cell-wall disruption conditions.

29. The method of claim 27, further comprising determining the level of lysis and/or the level of remaining intact cells present in the candidate antimicrobial compound-exposed target bacterial sample.

30. The method of claim 29, further comprising comparing the level of lysis and/or the level of remaining intact cells present in the candidate antimicrobial compound-exposed target bacterial sample to a reference level to score the sample as sensitive or resistant to the at least one candidate antimicrobial compound.

31. The method of claim 30, wherein if the level of lysis present in the candidate antimicrobial compound-exposed target bacterial sample is at or above a reference level and/or if the level of remaining intact cells is at or below a reference level then the at least one candidate compound is determined to be an antimicrobial compound.

32. A kit for use in for determining whether a target bacterium is susceptible to an antimicrobial compound, comprising:
at least one component of a cell-wall disruption condition and/or a means for creating a cell-wall disruption condition; and
a solid support for maintaining a sample comprising the target bacteria in the presence of the antimicrobial compound and for exposing the antimicrobial compound-exposed target bacterial sample to a cell-wall disruption condition.

33. The kit according to claim 32, further comprising a detectable label that selectively labels intact cells or selectively labels lysed cells.

34. The kit according to claim 32, wherein the at least one component of a cell-wall disruption condition and/or a means for creating a cell-wall disruption condition comprises at least one detergent.

35. The kit according to claim 34, wherein the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

36. The kit according to claim 32, further comprising a container comprising the antimicrobial compound.

37. A system for use in for determining whether a target bacterium is susceptible to an antimicrobial compound, comprising:
the kit of claim 32; and
means for determining whether the cell-wall disruption condition lyses target bacterial cells present in the antimicrobial compound-exposed target bacterial sample;
wherein the level of lysis and/or remaining intact cells is determined without determining lysis or non-lysis on a cell-by-cell basis.

38. The system of claim 37, wherein the target bacteria are not immobilized during the exposure to cell-wall disruption conditions.

39-40. (canceled)

41. The system according to claim 37, further comprising a detectable label that selectively labels intact cells or selectively labels lysed cells.

42. The system according to claim 37, wherein the at least one component of a cell-wall disruption condition and/or a means for creating a cell-wall disruption condition comprises at least one detergent.

43. The system according to claim 42, wherein the at least one detergent is selected from Brij 35, Brij 58, CHAPS, n-Dodecyl-beta-D-Maltoside, NP-40, Octyl-beta-Glucoside, Octyl-beta-Thioglucopyranoside, Sodium Dodecyl Sulfate-C12, Sodium Dodecyl Sulfate-Lauryl, Sodium Cholate, Sodium Deoxycholate, Triton X-100, Triton X-114, Tween 20, and Tween 80.

44. The system according to claim 37, further comprising a container comprising the antimicrobial compound.

45. The system according to claim 37, further comprising a positive control bacteria susceptible to the antimicrobial compound, wherein the positive control bacteria is lysed by a method comprising:
providing a sample comprising the positive control bacteria;
maintaining the sample in the presence of an antimicrobial compound to provide an antimicrobial compound-exposed positive control bacterial sample; and
exposing the antimicrobial compound-exposed positive control bacterial sample to a cell-wall disruption condition.

46. The system according to claim 37, further comprising a negative control bacteria resistant to the antimicrobial compound, wherein the negative control bacteria is not lysed by a method comprising:
providing a sample comprising the negative control bacteria;
maintaining the sample in the presence of an antimicrobial compound to provide an antimicrobial compound-exposed negative control bacterial sample; and
exposing the antimicrobial compound-exposed negative control bacterial sample to a cell-wall disruption condition.

47. The system according to claim 45, further comprising a negative control bacteria resistant to the antimicrobial compound, wherein the negative control bacteria is not lysed by a method comprising:
providing a sample comprising the negative control bacteria;
maintaining the sample in the presence of an antimicrobial compound to provide an antimicrobial compound-exposed negative control bacterial sample; and
exposing the antimicrobial compound-exposed negative control bacterial sample to the cell-wall disruption condition.

48. The system according to claim 37, further comprising a work station for application of a cell wall disruption condition to the sample.

49. The system according to claim 48, wherein the work station comprises a fluid dispenser for adding a cell wall disruption agent to the sample.

50. The system of claim 48, further comprising a fluid dispenser for adding an antibiotic to the sample.
